SEQUENCE LISTING (229 sequences)

SEQ ID NO: 1 - DNA, 11058 bp, homo sapiens
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 2 - protein, 3685 aa, homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 3 - DNA, 81 bp, homo sapiens
gctgccttgatatacacttttcaaaatgctttggtgggaagaagtagaggactgttgtaagtacaaagtaactaaaaatat

SEQ ID NO: 4 - DNA, 112 bp, homo sapiens
tttatttttttattttgcattttagatgaaagagaagatgttcaaaagaaaacattcacaaaatgggtaaatgcacaattttctaaggtaagaatggtttgttactttactt

SEQ ID NO: 5 - DNA, 143 bp, homo sapiens
ttgagtgtattttttttaatttcagtttgggaagcagcatattgagaacctcttcagtgacctacaggatgggaggcgcctcctagacctcctcgaaggcctgacagggcaaaaactggtatgtgacttatttttaagaaagt

SEQ ID NO: 6 - DNA, 128 bp, homo sapiens
gaacactcttttgttttgttctcagccaaaagaaaaaggatccacaagagttcatgccctgaacaatgtcaacaaggcactgcgggttttgcagaacaataatgtaagtagtaccctggacaaggtct

SEQ ID NO: 7 - DNA, 143 bp, homo sapiens
aatgttttacccctttctttaacaggttgatttagtgaatattggaagtactgacatcgtagatggaaatcataaactgactcttggtttgatttggaatataatcctccactggcaggtaagaatcctgatgaatggtttcc

SEQ ID NO: 8 - DNA, 223 bp, homo sapiens
tatgaaaatttatttccacatgtaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggtaagaagattactgagacattaaa

SEQ ID NO: 9 - DNA, 169 bp, homo sapiens
atgtgtgtatgtgtatgtgttttaggccagacctatttgactggaatagtgtggtttgccagcagtcagccacacaacgactggaacatgcattcaacatcgccagatatcaattaggcatagagaaactactcgatcctgaaggttggtaaatttctggactaccact

SEQ ID NO: 10 - DNA, 232 bp, homo sapiens
atgtgtagtgttaatgtgcttacagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacaggtaaagtgtgtaaaggacagctact

SEQ ID NO: 11 - DNA, 179 bp, homo sapiens
cactcccccaaacccttctctgcagatcacggtcagtctagcacagggatatgagagaacttcttcccctaagcctcgattcaagagctatgcctacacacaggctgcttatgtcaccacctctgaccctacacggagcccatttccttcacaggtctgtcaacatttactctctgttg

SEQ ID NO: 12 - DNA, 239 bp, homo sapiens
acacccaatttattttattgtgcagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggtaaactaaaacgttaatttacaaa

SEQ ID NO: 13 - DNA, 232 bp, homo sapiens
aattgttaacttccttctttgtcaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaagtaagtccttatttgtttttaatta

SEQ ID NO: 14 - DNA, 201 bp, homo sapiens
taataggcttctttcaaattttcagtttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtaggtgtatcttatgttgcgtgct

SEQ ID NO: 15 - DNA, 170 bp, homo sapiens
tcctttaaaacattttatctttcaggtgcttcaagaagatctagaacaagaacaagtcagggtcaattctctcactcacatggtggtggtagttgatgaatctagtggagatcacgcaactgctgctttggaagaacaacttaaggtcagattattttgcttagtaaact

SEQ ID NO: 16 - DNA, 152 bp, homo sapiens
ctgtgcttgattgtctcttctccaggtattgggagatcgatgggcaaacatctgtagatggacagaagaccgctgggttcttttacaagacatccttctcaaatggcaacgtcttactgaagaacaggtgtgtcatgtgtgagaaactagct

SEQ ID NO: 17 - DNA, 158 bp, homo sapiens
cttggaattctttaatgtcttgcagtgcctttttagtgcatggctttcagaaaaagaagatgcagtgaacaagattcacacaactggctttaaagatcaaaatgaaatgttatcaagtcttcaaaaactggccgtatgtactttctagctttcaatgg

SEQ ID NO: 18 - DNA, 230 bp, homo sapiens
tctgtgatctttcttgttttaacaggttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacaggttagtgataccaattatcatgcta

SEQ ID NO: 19 - DNA, 226 bp, homo sapiens
acctctgtttcaatacttctcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggtgagagcatcttaagcttttatct

SEQ ID NO: 20 - DNA, 174 bp, homo sapiens
tgacttttattttttgctgtcttaggttggatgttgatataactgaacttcacagctggattactcgctcagaagctgtgttgcagagtcctgaatttgcaatctttcggaaggaaggcaacttctcagacttaaaagaaaaagtcaatgtaggttatgcattaatttttatat

SEQ ID NO: 21 - DNA, 138 bp, homo sapiens
actcatctttgctctcatgctgcaggccatagagcgagaaaaagctgagaagttcagaaaactgcaagatgccagcagatcagctcaggccctggtggaacagatggtgaatggtaattacacgagttgatttagata

SEQ ID NO: 22 - DNA, 292 bp, homo sapiens
tatttaattatttttttctttctagagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggtaagaatctcttctccttccattt

SEQ ID NO: 23 - DNA, 231 bp, homo sapiens
ttactttccatactctatggcacaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaagtaagtaaaaagcctaaaatggcta

SEQ ID NO: 24 - DNA, 196 bp, homo sapiens
cattcttttttcccttttgataaagtttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggtctgtgaatatttgaatgtcaaaa

SEQ ID NO: 25 - DNA, 263 bp, homo sapiens
atgtatttaaaaaattgttttttaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcaggtaattcaagattttactttctacc

SEQ ID NO: 26 - DNA, 164 bp, homo sapiens
tgccttataacgggtctcgtttcagaatcacatacaaaccctgaagaaatggatggctgaagttgatgtttttctgaaggaggaatggcctgcccttggggattcagaaattctaaaaaagcagctgaaacagtgcagagtaagatttttatatgatgccttta

SEQ ID NO: 27 - DNA, 206 bp, homo sapiens
ggcttaaattgatttattttcttagcttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtatagacaatctctttcactgtgg

SEQ ID NO: 28 - DNA, 221 bp, homo sapiens
gttttgtttgtttgttttgtggaaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaaggtaaaaaaaaaaaaagaaaaactaa

SEQ ID NO: 29 - DNA, 233 bp, homo sapiens
taagagagcattctttatttttcagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagtcagttgcttttcttggtctttgt

SEQ ID NO: 30 - DNA, 185 bp, homo sapiens
tctgtgatatatatttctttcttaggaagtttgggcatgttggcatgagttattgtcatacttggagaaagcaaacaagtggctaaatgaagtagaatttaaacttaaaaccactgaaaacattcctggcggagctgaggaaatctctgaggtgctagatgtaagttgtaaattaagccaaatga

SEQ ID NO: 31 - DNA, 200 bp, homo sapiens
agtaattattgcaaatgtgtttcagtcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggtatgaagataagtgaaaaatctct

SEQ ID NO: 32 - DNA, 212 bp, homo sapiens
atacactcttattccttctttttaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccaggcaagtacatctgggaatcagcttc

SEQ ID NO: 33 - DNA, 161 bp, homo sapiens
actaataatgctatcctcccaacagaaaatccaatctgatttgacaagtcatgagatcagtttagaagaaatgaagaaacataatcaggggaaggaggctgcccaaagagtcctgtctcagattgatgttgcacaggtatatgttatttcagaaactaagg

SEQ ID NO: 34 - DNA, 224 bp, homo sapiens
gtgcctttttacactgtccttacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtggtatgtatttctggtggcaaatacg

SEQ ID NO: 35 - DNA, 206 bp, homo sapiens
tgttttgttttatgtttaaacttagaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtgtgtgcatgctgagaccacaaac

SEQ ID NO: 36 - DNA, 221 bp, homo sapiens
tacatttcattataattcttttcaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggtaaaacctatatcactgaaggtta

SEQ ID NO: 37 - DNA, 230 bp, homo sapiens
aaggtcaatgctctccttttcacaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggtaagagaaaaggctagaagctttt

SEQ ID NO: 38 - DNA, 179 bp, homo sapiens
catggtatgtctctgtacaattaaggaataccagaaacacatggaaacttttgaccagaatgtggaccacatcacaaagtggatcattcaggctgacacacttttggatgaatcagagaaaaagaaaccccagcaaaaagaagacgtgcttaaggtagcaaataaaatatgaaaagtaa

SEQ ID NO: 39 - DNA, 221 bp, homo sapiens
cctatctcttgctcatggaatatagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggtaggaagatctactccaaggtgga

SEQ ID NO: 40 - DNA, 173 bp, homo sapiens
aaagtagcactatctttttttttaggcctccattcctttgaaggaattggagcagtttaactcagatatacaaaaattgcttgaaccactggaggctgaaattcagcagggggtgaatctgaaagaggaagacttcaataaagatatggtaaattggttgtgataaaagtgtg

SEQ ID NO: 41 - DNA, 188 bp, homo sapiens
gactgtacttgttgtttttgatcagaatgaagacaatgagggtactgtaaaagaattgttgcaaagaggagacaacttacaacaaagaatcacagatgagagaaagcgagaggaaataaagataaaacagcagctgttacagacaaaacataatgctctcaaggtattagagctaaaattataatata

SEQ ID NO: 42 - DNA, 203 bp, homo sapiens
ttaataatgtctgcaccatgaacaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggtaatgttgttttagaatgtcaata

SEQ ID NO: 43 - DNA, 233 bp, homo sapiens
gccctgtattggttttgctcaataggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattgtgagttgttactggcaaacccacg

SEQ ID NO: 44 - DNA, 245 bp, homo sapiens
ttgttcttttgtatatctataccagcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaaggtaaaaccaaagcactttcattcgt

SEQ ID NO: 45 - DNA, 223 bp, homo sapiens
ctgttttaaaatttttatattacagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggtaggtaacacatatatttttcttg

SEQ ID NO: 46 - DNA, 198 bp, homo sapiens
ttgatccatatgcttttacctgcaggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggtaagtctttgatttgttttttcga

SEQ ID NO: 47 - DNA, 226 bp, homo sapiens
gttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggtagggcgacagatctaataggaat

SEQ ID NO: 48 - DNA, 198 bp, homo sapiens
aacaattttattcttctttctccaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaaggtaattttattttctcaaatccccc

SEQ ID NO: 49 - DNA, 200 bp, homo sapiens
acgttgttgcatttgtctgtttcagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggttagacattaaccatctcttccgt

SEQ ID NO: 50 - DNA, 236 bp, homo sapiens
tttttaaaatgtattttcctttcaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggtagggaactttttgctttaaatat

SEQ ID NO: 51 - DNA, 152 bp, homo sapiens
gcactatatgggttcttttccccaggaaactgaaatagcagttcaagctaaacaaccggatgtggaagagattttgtctaaagggcagcatttgtacaaggaaaaaccagccactcagccagtgaaggtaatgaagcaacctctagcaatat

SEQ ID NO: 52 - DNA, 159 bp, homo sapiens
taatgtgtatgcttttctgttaaagaggaagttagaagatctgagctctgagtggaaggcggtaaaccgtttacttcaagagctgagggcaaagcagcctgacctagctcctggactgaccactattggagcctgtaagtatactggatcccattctct

SEQ ID NO: 53 - DNA, 283 bp, homo sapiens
tttgcaaaaacccaaaatattttagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggtatgagaaaaaatgataaaagttg

SEQ ID NO: 54 - DNA, 168 bp, homo sapiens
tactaagggatatttgttcttacaggcaacaatgcaggatttggaacagaggcgtccccagttggaagaactcattaccgctgcccaaaatttgaaaaacaagaccagcaatcaagaggctagaacaatcattacggatcgaagtaagttttttaacaagcatgggac

SEQ ID NO: 55 - DNA, 262 bp, homo sapiens
atatttatttttccttttattctagttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaaggttagtatcaaagatacctttttaa

SEQ ID NO: 56 - DNA, 205 bp, homo sapiens
ttctctttctcataaaaatctatagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaaggtatgaattacattatttctaaaac

SEQ ID NO: 57 - DNA, 240 bp, homo sapiens
catctgaacatttggtcctttgcagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagtaagtcaggcatttccgctttagc

SEQ ID NO: 58 - DNA, 223 bp, homo sapiens
tattcttcttcctgctgtcctgtaggacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtaggaaaagatgtggagcaaaaag

SEQ ID NO: 59 - DNA, 207 bp, homo sapiens
atggtacgctgctgttctttttcaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggtaggacatttttaagcctcgtgcc

SEQ ID NO: 60 - DNA, 171 bp, homo sapiens
cacttcttttcatctcatttcacaggccttcaagagggaattgaaaactaaagaacctgtaatcatgagtactcttgagactgtacgaatatttctgacagagcagcctttggaaggactagagaaactctaccaggagcccagaggtaattgaatgtggaactataataa

SEQ ID NO: 61 - DNA, 319 bp, homo sapiens
aaaccttgtcatattgccaatttagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggtaccgtctacttctttgcttcagg

SEQ ID NO: 62 - DNA, 197 bp, homo sapiens
atttgcttttgactattgcacacaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtaagcacattgtaaacattgttgt

SEQ ID NO: 63 - DNA, 129 bp, homo sapiens
atcatttctctccttttcctcccaggtggccgtcgaggaccgagtcaggcagctgcatgaagcccacagggactttggtccagcatctcagcactttctttccagtaagtcattttcagcttttatcac

SEQ ID NO: 64 - DNA, 111 bp, homo sapiens
tctttttttcctcccttcttttcagcgtctgtccagggtccctgggagagagccatctcgccaaacaaagtgccctactatatcaagtaagttggaagtatcacattttta

SEQ ID NO: 65 - DNA, 112 bp, homo sapiens
tctttctttatgttttgtgttttagccacgagactcaaacaacttgctgggaccatcccaaaatgacagagctctaccagtctttaggtaaggacatggccatgtttcctcc

SEQ ID NO: 66 - DNA, 125 bp, homo sapiens
tgctctttgttttccctcttttcagctgacctgaataatgtcagattctcagcttataggactgccatgaaactccgaagactgcagaaggccctttgctgtaagtattggccagtatttgaaga

SEQ ID NO: 67 - DNA, 252 bp, homo sapiens
ttgtgattttatttgttttttgcagtggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgtacgtatggcatgtttttatttcc

SEQ ID NO: 68 - DNA, 136 bp, homo sapiens
tttctgctttgattcttcataataggggacgaacagggaggatccgtgtcctgtcttttaaaactggcatcatttccctgtgtaaagcacatttggaagacaagtacagatgtaagtcgtgtatattaatgctgta

SEQ ID NO: 69 - DNA, 208 bp, homo sapiens
ttgcaattttcttcttcctttgtagaccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgtaagttattcaccttctaggtaac

SEQ ID NO: 70 - DNA, 217 bp, homo sapiens
ttctctctccctcctgtctttgcaggctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtattaggaaccaaaaaaaaaatgt

SEQ ID NO: 71 - DNA, 162 bp, homo sapiens
cgtgtttgtttttgctctttatcaggtacaggagtctaaagcactttaattatgacatctgccaaagctgctttttttctggtcgagttgcaaaaggccataaaatgcactatcccatggtggaatattgcactccggtaagtttgacgccagcctgacgtg

SEQ ID NO: 72 - DNA, 187 bp, homo sapiens
gatctcaccatgatctcccttttagactacatcaggagaagatgttcgagactttgccaaggtactaaaaaacaaatttcgaaccaaaaggtattttgcgaagcatccccgaatgggctacctgccagtgcagactgtcttagagggggacaacatggaaacgtgagtagtagcaaaagcagaacac

SEQ ID NO: 73 - DNA, 89 bp, homo sapiens
caccacctcattttttgttttgcagtcccgttactctgatcaacttctggccagtagattctgcgtgagtactttttttgctgaagggt

SEQ ID NO: 74 - DNA, 116 bp, homo sapiens
actaatcacattttctgccttataggcctgcctcgtcccctcagctttcacacgatgatactcattcacgcattgaacattatgctagcaggtatgagactagttgtatgccaggc

SEQ ID NO: 75 - DNA, 116 bp, homo sapiens
aatgagcttttacgttttttatcaggctagcagaaatggaaaacagcaatggatcttatctaaatgatagcatctctcctaatgagagcatgtaagtatcccatctctttttacaa

SEQ ID NO: 76 - DNA, 209 bp, homo sapiens
caaaacctttgattttattttccagagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggtgagttttctttctagctttgtca

SEQ ID NO: 77 - DNA, 294 bp, homo sapiens
ttttttacttttttgatgccaataggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaagtgaggagagagatgggatttttac

SEQ ID NO: 78 - DNA, 174 bp, homo sapiens
ttctgttttcttttggatgacttagccccaggcagaggccaaagtgaatggcacaacggtgtcctctccttctacctctctacagaggtccgacagcagtcagcctatgctgctccgagtggttggcagtcaaacttcggactccatgggtaagtgtcctagctactctcagat

SEQ ID NO: 79 - DNA, 143 bp, homo sapiens
attatttgtttttgcttttattaaggtgaggaagatcttctcagtcctccccaggacacaagcacagggttagaggaggtgatggagcaactcaacaactccttccctagttcaagaggtaagctccaatacctagaagggac

SEQ ID NO: 80 - DNA, 82 bp, homo sapiens
cctcttcctctctctattattaaaggaagaaatacccctggaaagccaatgagagaggttagtgagattcaggctcacggcc

SEQ ID NO: 81 - DNA, 62 bp, homo sapiens
gtctttctttctctttgttttccaggacacaatgtaggaagtcttttccacatggcagatga

SEQ ID NO: 82 - DNA, 20 bp, Artificial Sequence
  Target sequence
tagaagatctgagctctgag

SEQ ID NO: 83 - DNA, 20 bp, Artificial Sequence
  Target sequence
agatctgagctctgagtgga

SEQ ID NO: 84 - DNA, 20 bp, Artificial Sequence
  Target sequence
tctgagctctgagtggaagg

SEQ ID NO: 85 - DNA, 20 bp, Artificial Sequence
  Target sequence
ccgtttacttcaagagctga

SEQ ID NO: 86 - DNA, 20 bp, Artificial Sequence
  Target sequence
aagcagcctgacctagctcc

SEQ ID NO: 87 - DNA, 20 bp, Artificial Sequence
  Target sequence
gctcctggactgaccactat

SEQ ID NO: 88 - DNA, 20 bp, Artificial Sequence
  Target sequence
ccctcagctcttgaagtaaa

SEQ ID NO: 89 - DNA, 20 bp, Artificial Sequence
  Target sequence
gtcagtccaggagctaggtc

SEQ ID NO: 90 - DNA, 20 bp, Artificial Sequence
  Target sequence
tagtggtcagtccaggagct

SEQ ID NO: 91 - DNA, 20 bp, Artificial Sequence
  Target sequence
gctccaatagtggtcagtcc

SEQ ID NO: 92 - DNA, 20 bp, Artificial Sequence
  Target sequence
tggccaaagacctccgccag

SEQ ID NO: 93 - DNA, 20 bp, Artificial Sequence
  Target sequence
gtggcagacaaatgtagatg

SEQ ID NO: 94 - DNA, 20 bp, Artificial Sequence
  Target sequence
tgtagatgtggcaaatgact

SEQ ID NO: 95 - DNA, 20 bp, Artificial Sequence
  Target sequence
cttggccctgaaacttctcc

SEQ ID NO: 96 - DNA, 20 bp, Artificial Sequence
  Target sequence
cagagaatatcaatgcctct

SEQ ID NO: 97 - DNA, 20 bp, Artificial Sequence
  Target sequence
ctgccactggcggaggtctt

SEQ ID NO: 98 - DNA, 20 bp, Artificial Sequence
  Target sequence
catttgtctgccactggcgg

SEQ ID NO: 99 - DNA, 20 bp, Artificial Sequence
  Target sequence
ctacatttgtctgccactgg

SEQ ID NO: 100 - DNA, 20 bp, Artificial Sequence
  Target sequence
catctacatttgtctgccac

SEQ ID NO: 101 - DNA, 20 bp, Artificial Sequence
  Target sequence
ataatcccggagaagtttca

SEQ ID NO: 102 - DNA, 20 bp, Artificial Sequence
  Target sequence
tatcatctgcagaataatcc

SEQ ID NO: 103 - DNA, 20 bp, Artificial Sequence
  Target sequence
tgttatcatgtggacttttc

SEQ ID NO: 104 - DNA, 20 bp, Artificial Sequence
  Target sequence
tgatatatcatttctctgtg

SEQ ID NO: 105 - DNA, 20 bp, Artificial Sequence
  Target sequence
tttatgaatgcttctccaag

SEQ ID NO: 106 - DNA, 21 bp, Artificial Sequence
  Target sequence
ttctccaggctagaagaacaa

SEQ ID NO: 107 - DNA, 21 bp, Artificial Sequence
  Target sequence
ctgctcttttccaggttcaag

SEQ ID NO: 108 - DNA, 21 bp, Artificial Sequence
  Target sequence
gtctgtttcagttactggtgg

SEQ ID NO: 109 - DNA, 21 bp, Artificial Sequence
  Target sequence
tccagtttcatttaattgttt

SEQ ID NO: 110 - DNA, 21 bp, Artificial Sequence
  Target sequence
cttatgggagcacttacaagc

SEQ ID NO: 111 - DNA, 21 bp, Artificial Sequence
  Target sequence
ttgcttcattaccttcactgg

SEQ ID NO: 112 - DNA, 21 bp, Artificial Sequence
  Target sequence
ttgtgtcaccagagtaacagt

SEQ ID NO: 113 - DNA, 21 bp, Artificial Sequence
  Target sequence
agtaaccacaggttgtgtcac

SEQ ID NO: 114 - DNA, 21 bp, Artificial Sequence
  Target sequence
ttcaaattttgggcagcggta

SEQ ID NO: 115 - DNA, 21 bp, Artificial Sequence
  Target sequence
caagaggctagaacaatcatt

SEQ ID NO: 116 - DNA, 21 bp, Artificial Sequence
  Target sequence
ttgtacttcatcccactgatt

SEQ ID NO: 117 - DNA, 21 bp, Artificial Sequence
  Target sequence
cttcagaaccggaggcaacag

SEQ ID NO: 118 - DNA, 21 bp, Artificial Sequence
  Target sequence
caacagttgaatgaaatgtta

SEQ ID NO: 119 - DNA, 21 bp, Artificial Sequence
  Target sequence
gccaagcttgagtcatggaag

SEQ ID NO: 120 - DNA, 21 bp, Artificial Sequence
  Target sequence
cttggtttctgtgattttctt

SEQ ID NO: 121 - DNA, 21 bp, Artificial Sequence
  Target sequence
tcatttcacaggccttcaaga

SEQ ID NO: 122 - DNA, 21 bp, Artificial Sequence
  Target sequence
cagaaatattcgtacagtctc

SEQ ID NO: 123 - DNA, 21 bp, Artificial Sequence
  Target sequence
caattacctctgggctcctgg

SEQ ID NO: 124 - RNA, 20 nt, Artificial Sequence
  gRNA
uagaagaucugagcucugag

SEQ ID NO: 125 - RNA, 20 nt, Artificial Sequence
  gRNA
agaucugagcucugagugga

SEQ ID NO: 126 - RNA, 20 nt, Artificial Sequence
  gRNA
ucugagcucugaguggaagg

SEQ ID NO: 127 - RNA, 20 nt, Artificial Sequence
  gRNA
ccguuuacuucaagagcuga

SEQ ID NO: 128 - RNA, 20 nt, Artificial Sequence
  gRNA
aagcagccugaccuagcucc

SEQ ID NO: 129 - RNA, 20 nt, Artificial Sequence
  gRNA
gcuccuggacugaccacuau

SEQ ID NO: 130 - RNA, 20 nt, Artificial Sequence
  gRNA
cccucagcucuugaaguaaa

SEQ ID NO: 131 - RNA, 20 nt, Artificial Sequence
  gRNA
gucaguccaggagcuagguc

SEQ ID NO: 132 - RNA, 20 nt, Artificial Sequence
  gRNA
uaguggucaguccaggagcu

SEQ ID NO: 133 - RNA, 20 nt, Artificial Sequence
  gRNA
gcuccaauaguggucagucc

SEQ ID NO: 134 - RNA, 20 nt, Artificial Sequence
  gRNA
uggccaaagaccuccgccag

SEQ ID NO: 135 - RNA, 20 nt, Artificial Sequence
  gRNA
guggcagacaaauguagaug

SEQ ID NO: 136 - RNA, 20 nt, Artificial Sequence
  gRNA
uguagauguggcaaaugacu

SEQ ID NO: 137 - RNA, 20 nt, Artificial Sequence
  gRNA
cuuggcccugaaacuucucc

SEQ ID NO: 138 - RNA, 20 nt, Artificial Sequence
  gRNA
cagagaauaucaaugccucu

SEQ ID NO: 139 - RNA, 20 nt, Artificial Sequence
  gRNA
cagagaauaucaaugccucu

SEQ ID NO: 140 - RNA, 20 nt, Artificial Sequence
  gRNA
cauuugucugccacuggcgg

SEQ ID NO: 141 - RNA, 20 nt, Artificial Sequence
  gRNA
cuacauuugucugccacugg

SEQ ID NO: 142 - RNA, 20 nt, Artificial Sequence
  gRNA
caucuacauuugucugccac

SEQ ID NO: 143 - RNA, 20 nt, Artificial Sequence
  gRNA
auaaucccggagaaguuuca

SEQ ID NO: 144 - RNA, 20 nt, Artificial Sequence
  gRNA
uaucaucugcagaauaaucc

SEQ ID NO: 145 - RNA, 20 nt, Artificial Sequence
  gRNA
uguuaucauguggacuuuuc

SEQ ID NO: 146 - RNA, 20 nt, Artificial Sequence
  gRNA
ugauauaucauuucucugug

SEQ ID NO: 147 - RNA, 20 nt, Artificial Sequence
  gRNA
uuuaugaaugcuucuccaag

SEQ ID NO: 148 - RNA, 21 nt, Artificial Sequence
  gRNA
uucuccaggcuagaagaacaa

SEQ ID NO: 149 - RNA, 21 nt, Artificial Sequence
  gRNA
cugcucuuuuccagguucaag

SEQ ID NO: 150 - RNA, 21 nt, Artificial Sequence
  gRNA
gucuguuucaguuacuggugg

SEQ ID NO: 151 - RNA, 21 nt, Artificial Sequence
  gRNA
uccaguuucauuuaauuguuu

SEQ ID NO: 152 - RNA, 21 nt, Artificial Sequence
  gRNA
cuuaugggagcacuuacaagc

SEQ ID NO: 153 - RNA, 21 nt, Artificial Sequence
  gRNA
uugcuucauuaccuucacugg

SEQ ID NO: 154 - RNA, 21 nt, Artificial Sequence
  gRNA
uugugucaccagaguaacagu

SEQ ID NO: 155 - RNA, 21 nt, Artificial Sequence
  gRNA
aguaaccacagguugugucac

SEQ ID NO: 156 - RNA, 21 nt, Artificial Sequence
  gRNA
uucaaauuuugggcagcggua

SEQ ID NO: 157 - RNA, 21 nt, Artificial Sequence
  gRNA
caagaggcuagaacaaucauu

SEQ ID NO: 158 - RNA, 21 nt, Artificial Sequence
  gRNA
uuguacuucaucccacugauu

SEQ ID NO: 159 - RNA, 21 nt, Artificial Sequence
  gRNA
cuucagaaccggaggcaacag

SEQ ID NO: 160 - RNA, 21 nt, Artificial Sequence
  gRNA
caacaguugaaugaaauguua

SEQ ID NO: 161 - RNA, 21 nt, Artificial Sequence
  gRNA
gccaagcuugagucauggaag

SEQ ID NO: 162 - RNA, 21 nt, Artificial Sequence
  gRNA
cuugguuucugugauuuucuu

SEQ ID NO: 163 - RNA, 21 nt, Artificial Sequence
  gRNA
ucauuucacaggccuucaaga

SEQ ID NO: 164 - RNA, 21 nt, Artificial Sequence
  gRNA
cagaaauauucguacagucuc

SEQ ID NO: 165 - RNA, 21 nt, Artificial Sequence
  gRNA
caauuaccucugggcuccugg

SEQ ID NO: 166 - RNA, 81 nt, Streptococcus pyogenes
guuuuagagcuagaaauagcaaguuaaaauaaggcuaguccguuaucaacuugaaaaaguggcaccgagucggugcuuuuu

SEQ ID NO: 167 - DNA, 7446 bp, Artificial Sequence
  Plasmid
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 168 - RNA, 20 nt, Artificial Sequence
  Cpf1 recognition sequence
uaauuucuacucuuguagau

SEQ ID NO: 169 - protein, 1391 aa, Artificial Sequence
  humanized Cas 9 from S. pyogenes (without NLS and without TAG)
GIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 170 - protein, 1397 aa, Artificial Sequence
  humanized Cas9 from S. pyogenes (with NLS and without TAG)
PKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 171 - protein, 1076 aa, Artificial Sequence
  Cas 9 from S. aureus (without NLS and without TAG)
GIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 172 - protein, 1083 aa, Artificial Sequence
  Cas 9 from S. aureus (with NLS and without TAG)
PKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 173 - DNA, 27 bp, Artificial Sequence
  oligonucleotide
gtcggaacaggagagcgcacgagggag

SEQ ID NO: 174 - DNA, 24 bp, Artificial Sequence
  oligonucleotide
ttcaccaaatggattaagatgttc

SEQ ID NO: 175 - DNA, 20 bp, Artificial Sequence
  oligonucleotide
actccccatatcccgttgtc

SEQ ID NO: 176 - DNA, 24 bp, Artificial Sequence
  oligonucleotide
gtttcaagtgatgagatagcaagt

SEQ ID NO: 177 - DNA, 24 bp, Artificial Sequence
  oligonucleotide
tatcagataacaggtaaggcagtg

SEQ ID NO: 178 - DNA, 21 bp, Artificial Sequence
  oligonucleotide
gagggcctatttcccatgatt

SEQ ID NO: 179 - DNA, 25 bp, Artificial Sequence
  oligonucleotide
cctccctaagcgctagggttacagg

SEQ ID NO: 180 - DNA, 20 bp, Artificial Sequence
  oligonucleotide
actccccatatcccgttgtc

SEQ ID NO: 181 - DNA, 25 bp, Artificial Sequence
  oligonucleotide
gtatttgaggtaccactgggccctc

SEQ ID NO: 182 - DNA, 25 bp, Artificial Sequence
  oligonucleotide
gccactgagctggacacacgaaatg

SEQ ID NO: 183 - DNA, 24 bp, Artificial Sequence
  oligonucleotide
gtcatgcttcagccttctccagac

SEQ ID NO: 184 - DNA, 24 bp, Artificial Sequence
  oligonucleotide
gtttatcccaggccagctttttgc

SEQ ID NO: 185 - DNA, 25 bp, Artificial Sequence
  oligonucleotide
ggctttgatttccctagggtccagc

SEQ ID NO: 186 - DNA, 25 bp, Artificial Sequence
  oligonucleotide
ggagaaggcaaattggcacagacaa

SEQ ID NO: 187 - DNA, 25 bp, Artificial Sequence
  oligonucleotide
gtaatccgaggtactccggaatgtc

SEQ ID NO: 188 - DNA, 24 bp, Artificial Sequence
  oligonucleotide
gtttcccctactccttcgtctgtc

SEQ ID NO: 189 - DNA, 25 bp, Artificial Sequence
  oligonucleotide
cactgggaaatcaggctgatgggtg

SEQ ID NO: 190 - DNA, 25 bp, Artificial Sequence
  oligonucleotide
gccaaggaaggagaattgcttgagg

SEQ ID NO: 191 - DNA, 24 bp, Artificial Sequence
  oligonucleotide
ggctcacggtatacctcacgatcc

SEQ ID NO: 192 - DNA, 24 bp, Artificial Sequence
  oligonucleotide
cctcctcacagataactccctttg

SEQ ID NO: 193 - DNA, 26 bp, Artificial Sequence
  oligonucleotide
cactgcgcctggccaggaatttttgc

SEQ ID NO: 194 - DNA, 26 bp, Artificial Sequence
  oligonucleotide
caatagaagcaaagacaaggtagttg

SEQ ID NO: 195 - DNA, 25 bp, Artificial Sequence
  oligonucleotide
gcacaaactgatttatgcatggtag

SEQ ID NO: 196 - protein, 35 aa, Artificial sequence
  Synthetic construct
HHHHHHKWKLFKKIGAVLKVLTTGYARAAARQARA

SEQ ID NO: 197 - protein, 41 aa, Artificial sequence
  Synthetic construct
HHHHHHKWKLFKKIGAVLKVLTTGYARAAARQARAHHHHHH

SEQ ID NO: 198 - protein, 37 aa, Artificial sequence
  Synthetic construct
HHHHHHLLKLWSRLLKLWTQGRRLKAKRAKAHHHHHH

SEQ ID NO: 199 - DNA, 485 bp, Artificial sequence
  Synthetic construct
ttcaccaaatggattaagatgttcatgaattatcttcaaagtgttaatcgaataagtaatgtgtatgcttttctgttaaagaggaagttagaagatctgagctcttcttggagaagcattcataaaaggtatgaattacattatttctaaaactactgttggctgtaataatggggtggtgaaactggatggaccatgaggatttgtttttccaatccagctaaactggagcttgggagggttcaagacgataaataccaactaaactcacggacttggctcagacttctattttaaaaacgaggaacataagatctcatttgcccgctgtcacaaaagtagtgacataaccaagagattaaacaaaaagcaaaatactgatttatagctagaagagccatttatcagtctactttgataactctatccaaaggaatatctttctatctcatcatggcgcacactgccttacctgttatctgata

SEQ ID NO: 200 - DNA, 999 bp, Artificial sequence
  Synthetic construct
tatgcgcgaccgagttgctcttgccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgactcgagaattctgacgtcattattatcagttcaccaaatggattaagatgttcatgaattatcttcaaagtgttaatcgaataagtaatgtgtatgcttttctgttaaagaggaagttagaagatctgagctcttcttggagaagcattcataaaaggtatgaattacattatttctaaaactactgttggctgtaataatggggtggtgaaactggatggaccatgaggatttgtttttccaatccagctaaactggagcttgggagggttcaagacgataaataccaactaaactcacggacttggctcagacttctattttaaaaacgaggaacataagatctcatttgcccgctgtcacaaaagtagtgacataaccaagagattaaacaaaaagcaaaatactgatttatagctagaagagccatttatcagtctactttgataactctatccaaaggaatatctttctatctcatcatggcgcacactgccttacctgttatctgataatcatggtttttacctcctgaattcggatccctcgagcgatacagcgagtcgctctttgtggaggtgacgattacctaacaaaatcggac

SEQ ID NO: 201 - DNA, 41 bp, Artificial sequence
  Synthetic construct
taatacgactcactataagaagatctgagctctgaggtttt

SEQ ID NO: 202 - DNA, 43 bp, Artificial sequence
  Synthetic construct
taatacgactcactataagagaatatcaatgcctctgttttag

SEQ ID NO: 203 - DNA, 23 bp, Artificial sequence
  Synthetic construct
aaaaaagcaccgactcggtgcca

SEQ ID NO: 204 - DNA, 25 bp, Artificial sequence
  Synthetic construct
ctgtgatgtgaggacatataaagac

SEQ ID NO: 205 - DNA, 25 bp, Artificial sequence
  Synthetic construct
tcaatgtagggaaggaaatatggca

SEQ ID NO: 206 - DNA, 19 bp, Artificial sequence
  Synthetic construct
gaacttctatttaattttg

SEQ ID NO: 207 - DNA, 20 bp, Artificial sequence
  Synthetic construct
atttcaggtaagccgaggtt

SEQ ID NO: 208 - DNA, 20 bp, Artificial sequence
  Synthetic construct
tcttaataatgtttcactgt

SEQ ID NO: 209 - DNA, 20 bp, Artificial sequence
  Synthetic construct
ataatttctattatattaca

SEQ ID NO: 210 - DNA, 20 bp, Artificial sequence
  Synthetic construct
tttcattcatatcaagaaga

SEQ ID NO: 211 - DNA, 20 bp, Artificial sequence
  Synthetic construct
atagtttaaaggccaaacct

SEQ ID NO: 212 - DNA, 20 bp, Artificial sequence
  Synthetic construct
tgtgaaaaaatatagtttaa

SEQ ID NO: 213 - DNA, 20 bp, Artificial sequence
  Synthetic construct
cgaaaatttcaggtaagccg

SEQ ID NO: 214 - RNA, 19 nt, Artificial sequence
  Synthetic construct
gaacuucuauuuaauuuug

SEQ ID NO: 215 - RNA, 20 nt, Artificial sequence
  Synthetic construct
auuucagguaagccgagguu

SEQ ID NO: 216 - RNA, 20 nt, Artificial sequence
  Synthetic construct
ucuuaauaauguuucacugu

SEQ ID NO: 217 - RNA, 20 nt, Artificial sequence
  Synthetic construct
auaauuucuauuauauuaca

SEQ ID NO: 218 - RNA, 20 nt, Artificial sequence
  Synthetic construct
uuucauucauaucaagaaga

SEQ ID NO: 219 - RNA, 20 nt, Artificial sequence
  Synthetic construct
auaguuuaaaggccaaaccu

SEQ ID NO: 220 - RNA, 20 nt, Artificial sequence
  Synthetic construct
ugugaaaaaauauaguuuaa

SEQ ID NO: 221 - RNA, 20 nt, Artificial sequence
  Synthetic construct
cgaaaauuucagguaagccg

SEQ ID NO: 222 - DNA, 22 bp, Artificial sequence
  Synthetic construct
tgcctggagaaagggtttttgt

SEQ ID NO: 223 - DNA, 26 bp, Artificial sequence
  Synthetic construct
gaacatgtcttatcagtcaagagatc

SEQ ID NO: 224 - DNA, 25 bp, Artificial sequence
  Synthetic construct
tcaatgtagggaaggaaatatggca

SEQ ID NO: 225 - DNA, 25 bp, Artificial sequence
  Synthetic construct
aactatctgagtgacactgtgaagg

SEQ ID NO: 226 - DNA, 25 bp, Artificial sequence
  Synthetic construct
ctgtgatgtgaggacatataaagac

SEQ ID NO: 227 - DNA, 25 bp, Artificial sequence
  Synthetic construct
cagacaatccaagaaggtatgacac

SEQ ID NO: 228 - DNA, 427 bp, Homo sapiens
ctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaaga

SEQ ID NO: 229 - DNA, 217 bp, Homo sapiens
aataaacttcgaaaatttcaggtaagccgaggtttggcctttaaactatattttttcacatagcaattaattggaaaatgtgatgggaaacagatattttacccagagtccttcaaagatattgatgatatcaaaagccaaatctatttcaaaggattgcaacttgcctatttttcctatgaaaacagtaatgtgtcataccttcttggattgtctg

(19) United States
(12) Patent Application Publication
(10) Pub. No.: US 2019/0248854 A1
(43) Pub. Date: Aug. 15, 2019

(54) METHODS OF MODIFYING THE DYSTROPHIN GENE AND RESTORING DYSTROPHIN EXPRESSION AND USES THEREOF

(71) Applicant: UNIVERSITÉ LAVAL, QUÉBEC (CA)

(72) Inventors: JACQUES P. TREMBLAY, QUÉBEC (CA); JEAN-PAUL IYOMBE-ENGEMBE, QUÉBEC (CA); PIERRE CHAPDELAINE, SAINT-ROMUALD (CA); DANIEL AGUDELO, QUÉBEC (CA); BENJAMIN DUCHÊNE, CHATEAUGIRON (FR)

(21) Appl. No.: 16/335,743

(22) PCT Filed: Sep. 21, 2017
(86) PCT No.: PCT/CA2017/051109

§ 371 Date: Mar. 22, 2019

Related U.S. Application Data
(60) Provisional application No. 62/474,827, filed on Mar. 22, 2017.

(30) Foreign Application Priority Data
Sep. 23, 2016 (CA) PCT/CA2016/051117

(51) Int. Cl. C07K 14/47; C12N 15/86; A61K 48/00; C12N 15/90; C12N 9/22; A61P 21/00; A01K 67/027

(52) U.S. Cl. CPC C07K 14/4708; C12N 15/86; A61K 48/0016; A61K 48/0075; A61K 48/0058; A01K 2267/0306; C12N 9/22; A61P 21/00; A01K 67/0278; A01K 2227/105; C12N 15/907

(57) ABSTRACT

Methods for modifying a dystrophin gene are disclosed, for restoring dystrophin expression within a cell having an endogenous frameshift or nonsense mutation within the dystrophin gene. The methods comprise introducing a first cut within an exon en or intron of the dystrophin gene creating a first exon end or intron end, wherein said first cut is located upstream of the endogenous frameshift or nonsense mutation; and introducing a second cut within an exon or intron of the dystrophin gene creating a second exon end or intron end, wherein said second cut is located downstream of the frameshift or nonsense mutation. Upon joining/ligation of said first and second exon ends or intron ends a hybrid exon or intron junction is created and dystrophin expression is restored, as the correct reading frame is restored. Reagents and uses of the method are also disclosed, for example to treat a subject suffering from muscular dystrophy.

### CROSS REFERENCE TO RELATED APPLICATIONS

This application claims priority, of PCT Application No. PCT CA2016/051117 filed on Sep. 23, 2016 and of U.S. provisional application Ser. No. 62/474,827, filed on Mar. 22, 2017, which are incorporated herein by reference in their entirety.

### SEQUENCE LISTING

This application contains a Sequence Listing in computer readable form entitled “11229_375_SL_ST25.txt”, created on Sep. 19, 2017 and having a size of about 155 KB. The computer readable form is incorporated herein by reference.

### FIELD OF THE INVENTION

The present invention relates to the targeted modification of an endogenous mutated dystrophin gene to restore dystrophin expression in mutated cells, such as cells of subjects suffering from Muscular Dystrophy (MD), such as Duchenne MD (DMD) and Becker MD (BMD). More specifically, the present invention is concerned with correcting the reading frame of a mutated dystrophin gene by targeting exon or intron sequences close to the endogenous mutation. The present invention also relates to such modified forms of dystrophin.

### BACKGROUND OF THE INVENTION

Duchenne Muscular Dystrophy (DMD) is a monogenic hereditary disease linked to the X chromosome, which affects one in about 3500 male births [1]. The cause of the disease is the inability of the body to synthesize the dystrophin (DYS) protein, which plays a fundamental role in maintaining the integrity of the sarcolemma [2, 3]. The absence of this protein is secondary to a mutation of the DYS gene [4]. The most frequently encountered mutations, found in over 60% of DMD patients, are deletions of one or more exons in the region between exons 45 and 55, called the hot region of DYS gene [5]. Most of these deletions induce a codon frame-shift of the mRNA transcript leading to the production of a truncated DYS protein. Since the latter is rapidly degraded, the absence of DYS at the sarcolemma increases its fragility and leads to muscle weakness characteristic of DMD. In some cases deletions result in the milder Becker Muscular Dystrophy (BMD) phenotype [6]. For DMD patients, skeletal muscular weaknesses will unfortunately lead to death, between 18 and 30 years of age [7, 8], while some BMD patients can have a normal life expectancy [6]. To date, there is no cure for DMD and BMD.

The identification of the molecular basis for the DMD and BMD phenotypes established the foundation for DMD gene therapy [9-13]. Different strategies for DMD gene therapy are currently under development. Since the 2.4-Mb DYS gene contains 79 exons and encodes a 14 kb mRNA [14, 15], it is difficult to develop a gene therapy to deliver efficiently the full-length gene or even its cDNA in muscle precursor cells in vitro or in muscle fibers in vivo.

An alternative to gene replacement is to modify the DYS mRNA or the DYS gene itself directly within cells. Correction of the reading frame of the mRNA can be obtained by exon skipping using a synthetic antisense oligonucleotide (AON) interacting in with the primary transcript with the splice donor or spice acceptor of the exon, which precedes or follows the patient deletion [20-28]. Unfortunately, this therapeutic approach is facing a number of difficulties associated with the lifetime use of AONs [29]. Further, the AONs act only on the mRNA, thus the DMD patients treated with this approach are required to receive this treatment for life, which is very expensive and increases the risks of complications.

Thus, there remains a need for novel therapeutic approaches for restoring dystrophin expression in cells.

The present description refers to a number of documents, the content of which is herein incorporated by reference in their entirety.

### SUMMARY OF THE INVENTION

The present invention relates to restoring the correct reading frame of a mutant DYS gene, which may be used as a new therapeutic approach for treating muscular dystrophy (MD) (e.g., DMD). This can be done directly on the cells of a subject suffering from MD. This approach is based on the permanent restoration of the DYS reading frame by generating additional mutations (e.g., deletion) upstream and downstream of an endogenous frameshift or nonsense mutation. These engineered upstream and downstream mutations may be within an exon containing the endogenous frameshift or nonsense mutation, and/or may be within exons or introns flanking the endogenous frameshift or nonsense mutation (e.g., exons or introns upstream and downstream from the frameshift or nonsense mutation). In a first aspect, by targeting exons (as opposed to introns) as the sites to introduce these engineered mutations, it is possible to restore the reading frame of the DYS gene in cells to produce a modified dystrophin protein having the smallest possible deletion while still retaining sufficient dystrophin protein function. Alternatively, an entire exon comprising an endogenous frameshift or nonsense mutation the may be deleted to restore the dystrophin reading frame. Notably, by specifically selecting the deletion to be introduced into the dystrophin gene comprising the endogenous frameshift or nonsense mutation, applicants were able to maintain the configuration of normal spectrin-like repeats in the modified dystrophin protein where hydrophobic amino acids are localized in position “a” and “d” of the heptad motif, thereby generating a functional (albeit shorter) dystrophin protein.

For the treatment of MD such as DMD, agents for introducing gene modifications need to be delivered effectively into cells for the treatment to be efficient. AAV vectors are currently the vehicle of choice for delivery of gene modifying components into cells. However, sustained expression of the CRISPR nuclease (e.g., Cas9 nuclease) may increase off-target mutations. In an effort to identify effective alternative delivery methods, Applicants explored various methods/agents for carrying sgRNAs/CRISPR nuclease combination into cells. It was found that ribonucleoprotein sgRNA/Cas9 complex can be effectively transduced in vitro in myoblasts and in vivo in muscle fibers using different delivery agents, including new cell penetrating peptides called Feldan Shuttles (FS). This delivery method reduces the life-time of the CRISPR nuclease in cells and may thus be used to reduce off-target mutations making the treatment more secure. Furthermore, it was found that among the polypeptide-based shuttles tested, some carriers were surprisingly more effective than others.

Accordingly, in accordance with the present invention, there is provided a method of modifying a dystrophin gene and restoring the correct reading frame for dystrophin expression within a cell having an endogenous frameshift or nonsense mutation within the dystrophin (DYS) gene, the method comprising:

a) introducing a first cut within an exon or intron of the DYS gene creating a first exon end or intron end, wherein said first cut is located upstream of the endogenous frameshift or nonsense mutation;
b) introducing a second cut within an exon or an intron of the DYS gene creating a second exon end or intron end, wherein said second cut is located downstream of the frameshift or nonsense mutation;

wherein upon ligation of said first and second exon ends or said first and second intron ends, a modified dystrophin gene comprising a hybrid exon or a hybrid intron is created and dystrophin expression is restored.

In an embodiment, the first and second cuts are within one or more exons, and are not within an intron, of the dystrophin gene (although a gRNA or a portion thereof may bind to an intron, in particular in an intronic region flanking an exon, as long as the resulting cut is in an exon). As a result, following the introduction of the first and second cuts, the first exon end is ultimately joined or ligated to the second exon end, creating a hybrid, fusion exon and at the same time restoring the correct reading frame, allowing transcription to the end of the dystrophin gene, producing a truncated dystrophin protein (at least lacking the portion comprising the endogenous frameshift or nonsense mutation) due to the removal of a portion of the gene by the first and second cuts (e.g., a first cut in the first exon upstream of the endogenous mutation and a second cut in the second exon downstream of the endogenous mutation). Depending on the site of the first and second cuts, ligation of exon ends may lead to the introduction of a new codon in the amino acid sequence of the dystrophin protein. Preferably, the location of the cuts to be introduced are specifically selected such that the configuration of spectrin-like repeats in the modified dystrophin protein is maintained (i.e., hydrophobic amino acids in the spectrin-like repeats are localized in position “a” and “d” of the heptad motif, as known in the art; see for example FIG. 16).

In another embodiment, the first and second cuts are within introns flanking the endogenous frameshift or nonsense mutation of the dystrophin gene (although a gRNA or a portion thereof may bind to an exon, in particular in an exonic region flanking an intron, as long as the resulting cut is in an intron). As a result, following the introduction of the first and second cuts, the first intron end is ultimately joined or ligated to the second intron end, creating a hybrid, fusion intron, deleting the exon(s) located between the introns and at the same time restoring the correct reading frame, allowing transcription to the end of the dystrophin gene, producing a truncated dystrophin protein (at least lacking the portion comprising the endogenous frameshift or nonsense mutation) due to the removal of a portion of the gene by the first and second cuts (e.g., a first cut in the first intron upstream of the endogenous mutation and a second cut in the second intron downstream of the endogenous mutation). Preferably, the location of the cuts to be introduced are specifically selected such that the configuration of spectrin-like repeats in the modified dystrophin protein is maintained (i.e., hydrophobic amino acids in the spectrin-like repeats are localized in position “a” and “d” of the heptad motif, as known in the art; see for example FIG. 16). In embodiments, one or more complete spectrin-like repeats (R1, R2, R3, R4 . . . R15, R16, R17′ R18, R19, R20, R21, R22, R23, R24 or any combination thereof) are removed by the deletion of one or more exons.

In an embodiment, said first and second cuts are introduced by providing a cell with i) a CRISPR nuclease (e.g., Cas9 nuclease); and ii) a pair of gRNAs consisting of a) a first gRNA which binds to an exon or intron sequence of the DYS gene located upstream of the endogenous frameshift or nonsense mutation for introducing a first cut; b) a second gRNA which binds to an exon or intron sequence of the DYS gene located downstream of the endogenous frameshift or nonsense mutation for introducing the second cut.

In an embodiment, the endogenous frameshift or nonsense mutation is located in one or more exons selected from exons 45-58 of the dystrophin gene.

In embodiments, the first cut is within exon 45, 46, 47, 48, 49 or 50 and the second cut is within exon 51, 52, 53, 54, 55, 56, 57 or 58, of the dystrophin gene.

In embodiments, the first cut is within exon 45 and the second cut is within exon 51, 52, 53, 54, 55, 56, 57 or 58, of the dystrophin gene.

In embodiments, the first cut is within exon 46 and the second cut is within exon 51, 52, 53, 54, 55, 56, 57 or 58, of the dystrophin gene.

In embodiments, the first cut is within exon 47 and the second cut is within exon 51, 52, 53, 54, 55, 56, 57 or 58, of the dystrophin gene.

In embodiments, the first cut is within exon 48 and the second cut is within exon 51, 52, 53, 54, 55, 56, 57 or 58, of the dystrophin gene.

In embodiments, the first cut is within exon 49 and the second cut is within exon 51, 52, 53, 54, 55, 56, 57 or 58, of the dystrophin gene.

In embodiments, the second cut is within exon 51 and the first cut is within exon 45, 46, 47, 48 or 49, of the dystrophin gene.

In embodiments, the second cut is within exon 52 and the first cut is within exon 45, 46, 47, 48 or 49, of the dystrophin gene.

In embodiments, the second cut is within exon 53 and the first cut is within exon 45, 46, 47, 48 or 49, of the dystrophin gene.

In embodiments, the second cut is within exon 54 and the first cut is within exon 45, 46, 47, 48 or 49, of the dystrophin gene.

In embodiments, the second cut is within exon 55 and the first cut is within exon 45, 46, 47, 48 or 49, of the dystrophin gene.

In embodiments, the second cut is within exon 56 and the first cut is within exon 45, 46, 47, 48 or 49, of the dystrophin gene.

In embodiments, the second cut is within exon 57 and the first cut is within exon 45, 46, 47, 48 or 49, of the dystrophin gene.

In embodiments, the second cut is within exon 58 and the first cut is within exon 45, 46, 47, 48 or 49, of the dystrophin gene.

In an embodiment, the first cut is within exon 50 and the second cut is within exon 54, of the dystrophin gene.

In an embodiment, the first cut is within exon 46 and the second cut is within exon 51, of the dystrophin gene.

In an embodiment, the first cut is within exon 46 and the second cut is within exon 53, of the dystrophin gene.

In an embodiment, the first cut is within exon 47 and the second cut is within exon 52, of the dystrophin gene.

In an embodiment, the first cut is within exon 49 and the second cut is within exon 52, of the dystrophin gene.

In an embodiment, the first cut is within exon 49 and the second cut is within exon 53, of the dystrophin gene.

In an embodiment, the first cut is within exon 47 and the second cut is within exon 58, of the dystrophin gene.

In embodiments, the first cut is within intron 45-58 and the second cut is within intron 46-59, of the dystrophin gene.

In embodiments, the first cut is within intron 45, 46, 47, 48, 49 or 50 and the second cut is within intron 51, 52, 53, 54, 55, 56, 57, 58 or 59, of the dystrophin gene.

In embodiments, the first cut is within intron 45 and the second cut is within intron 51, 52, 53, 54, 55, 56, 57 or 58, of the dystrophin gene.

In embodiments, the first cut is within intron 46 and the second cut is within intron 51, 52, 53, 54, 55, 56, 57 or 58, of the dystrophin gene.

In embodiments, the first cut is within intron 47 and the second cut is within intron 51, 52, 53, 54, 55, 56, 57 or 58, of the dystrophin gene.

In embodiments, the first cut is within intron 48 and the second cut is within intron 51, 52, 53, 54, 55, 56, 57 or 58, of the dystrophin gene.

In embodiments, the first cut is within intron 49 and the second cut is within intron 51, 52, 53, 54, 55, 56, 57 or 58, of the dystrophin gene.

In embodiments, the second cut is within intron 51 and the first cut is within intron 45, 46, 47, 48 or 49, of the dystrophin gene.

In embodiments, the second cut is within intron 52 and the first cut is within intron 45, 46, 47, 48 or 49, of the dystrophin gene.

In embodiments, the second cut is within intron 53 and the first cut is within intron 45, 46, 47, 48 or 49, of the dystrophin gene.

In embodiments, the second cut is within intron 54 and the first cut is within intron 45, 46, 47, 48 or 49, of the dystrophin gene.

In embodiments, the second cut is within intron 55 and the first cut is within intron 45, 46, 47, 48 or 49, of the dystrophin gene.

In embodiments, the second cut is within intron 56 and the first cut is within intron 45, 46, 47, 48 or 49, of the dystrophin gene.

In embodiments, the second cut is within intron 57 and the first cut is within intron 45, 46, 47, 48 or 49, of the dystrophin gene.

In embodiments, the second cut is within intron 58 and the first cut is within intron 45, 46, 47, 48 or 49, of the dystrophin gene.

In an embodiment, the first cut is within intron 22 and the second cut is within intron 23, of the dystrophin gene.

In embodiments, the first and second cuts generate a hybrid exon. In embodiments. the first and second cuts generate a hybrid intron.

In an embodiment, the hybrid exon generated by the method of the present invention has a nucleic acid sequence as set forth in FIG. 5a, FIG. 9a(ii) or FIG. 17. In an embodiment, the first and second gRNAs (the gRNA pair), together with a CRISPR nuclease (e.g., SaCas9 or SpCas9), allow to generate a hybrid exon from (i) exon 46 and exon 51 (hybrid exon 46-51); (ii) exon 46 and exon 53 (hybrid exon 46-53); (iii) exon 47 and exon 52 (hybrid exon 47-52); (iv) exon 49 and exon 52 (hybrid exon 49-52); (v) exon 49 and exon 53 (hybrid exon 49-53); or (vi) exon 47 and exon 58 (hybrid exon 47-58).

In an embodiment, the above-noted method generates a modified dystrophin gene encoding a modified dystrophin protein comprising a hybrid spectrin-like repeat (SLR) comprising a portion of a first SLR and a portion of second SLR and a hybrid SLR junction. In the hybrid SLR, the normal (wild-type) configuration of the SLR is maintained where hydrophobic amino acids are localized in position “a” and “d” of the heptad motif.

In an embodiment, the modified dystrophin protein generated by the method of the present invention comprises the following hybrid spectrin-like repeat (SLR): (i) a hybrid SLR comprising a portion of SLR 17 and a portion of SLR 19 (SLR 17-19); (ii) a hybrid SLR comprising a portion of SLR 17 and a portion of SLR 20 (SLR 17-20); (iii) a hybrid SLR comprising a portion of SLR 18 and a portion of SLR 20 (SLR 18-20); (iv) a hybrid SLR comprising a portion of SLR 18 and a portion of SLR 21 (SLR 18-21); (v) a hybrid SLR comprising a portion of SLR 19 and a portion of SLR 20 (SLR 19-20); or (vi) a hybrid SLR comprising a portion of SLR 18 and a portion of SLR 23 (SLR 18-23).

In an embodiment, the hybrid SLR has a hybrid SLR junction selected from the hybrid SLR junctions set forth in FIGS. 5a, 5b and 16b.

CRISPR nucleases and gRNAs of the present invention may be introduced into the cells using any useful methods known in the art.

In an embodiment, the first gRNA, the second gRNA and/or the CRISPR nuclease are delivered into the cell using one or more adeno-associated virus (AAV) vectors.

In another embodiment the first gRNA, the second gRNA and/or the CRISPR nuclease are delivered into the cell using one or more polypeptide-based shuttles (e.g., Feldan Shuttle).

In an embodiment, the one or more polypeptide-based shuttle comprises: (i) a polypeptide-based shuttle having amino acid sequence HHHHHHKWKLFKKIGAVLKVLTTGYARAAARQARA (FSA, SEQ ID NO: 196); (ii) a polypeptide-based shuttle having amino acid sequence HHHHHHKWKLFKKIGAVLKVLTTGYARAAARQARAHHHHHH (FSB, SEQ ID NO: 197); (iii) a polypeptide-based shuttle having amino acid sequence HHHHHHLLKLWSRLLKLWTQGRRLKAKRAKAHHHHHH (FSC, SEQ ID NO: 198); or (iv) any combination of at least two of (i), (ii) and (iii).

In an embodiment, the CRISPR nuclease used in accordance with the present invention is a Cas9 nuclease derived from Staphylococcus aureus (saCas9) or from Streptococcus pyogenes (SpCas9).

Also provided is a gRNA pair for use in the method of the present invention (e.g., for restoring dystrophin expression in a cell comprising an endogenous frameshift or nonsense mutation within the dystrophin (DYS) gene, or for treating muscular dystrophy). In an embodiment, the pair consists of a first gRNA and a second gRNA, wherein the first gRNA comprises a first target sequence upstream of the endogenous frameshift or nonsense mutation (i.e., the gRNA binds to the complementary strand (the opposite strand) of the first target sequence) and can direct a nuclease-mediated first cut in an exon or intron sequence of the DYS gene located upstream of the endogenous frameshift or nonsense mutation and wherein the second gRNA comprises a second target sequence downstream of the endogenous frameshift or nonsense mutation (i.e., it binds to the complementary sequence (the opposite strand) of the second target sequence) and can direct a nuclease-mediated second cut in an exon or intron sequence of the DYS gene located downstream of the endogenous frameshift or nonsense mutation.

In an embodiment, each of the first target sequence and the second target sequence of the first and second gRNAs is adjoining a PAM sequence in the DYS gene set forth in Table 3, Table 6, Table 8, FIG. 1 or FIG. 11. In embodiments, the target sequence does not consist of the target sequence of gRNAs 20, 22, 23 or 26 set forth in Table 8.

In embodiments, the target sequence of the first gRNA and/or second gRNA spans an intron-exon junction (i.e., the target sequence comprises both intronic and exonic sequences).

In an embodiment, each of the first target sequence and the second target sequence of the first and second gRNAs comprises or consists of a target sequence selected from the target sequences set forth in listed in Table 3, 6 or 8 or shown in FIG. 11.

In an embodiment, the first and second target sequences are each independently 10-40 nucleotides in length.

In an embodiment, the gRNA pair is selected from a gRNA pair set forth in FIG. 4, 13, 15, 17, 21 or 22 or Table 4 or 7. In embodiments, the gRNA pair is not selected from gRNAs 20 and 23 or 20 and 22 set forth in Table 8.

In an embodiment, the first gRNA and the second gRNA in the gRNA pair are selected from the gRNAs listed in Table 3, 6 or 8 or shown in FIG. 11. In an embodiment, the first gRNA or the second gRNA in the gRNA pair does not have a target sequence consisting of the target sequence of gRNA 20, 22, 23 or 26 set forth in Table 8. In an embodiment, the first gRNA of the gRNA pair has the target sequence which comprises prises or consists of the sequence AGATCTGAGCTCTGAGTGGA (SEQ ID NO: 83).

In an embodiment, the first gRNA of the gRNA pair has the target sequence which comprises prises or consists of the sequence GTCTGTTTCAGTTACTGGTGG (SEQ ID NO: 108).

In an embodiment, the first gRNA of the gRNA pair has the target sequence which comprises prises or consists of the sequence CTTATGGGAGCACTTACAAGC (SEQ ID NO: 110)

In an embodiment, the second gRNA of the gRNA pair has the target sequence which comprises prises or consists of the sequence GTGGCAGACAAATGTAGATG (SEQ ID NO: 93).

In an embodiment, the second gRNA of the gRNA pair has the target sequence which comprises prises or consists of the sequence TCATTTCACAGGCCTTCAAGA (SEQ ID NO: 121).

In an embodiment, the second gRNA of the gRNA pair has the target sequence which comprises prises or consists of the sequence CAATTACCTCTGGGCTCCTGG (SEQ ID NO: 123).

In an embodiment, the gRNA pair consists of a first gRNA having a first target sequence which is GTCTGTTTCAGTTACTGGTGG (SEQ ID NO: 108) and a second gRNA having a second target sequence which is TCATTTCACAGGCCTTCAAGA (SEQ ID NO: 121).

In an embodiment, the gRNA pair consists of a first gRNA having a first target sequence which is CTTATGGGAGCACTTACAAGC (SEQ ID NO: 110) and a second gRNA having a second target sequence which is CAATTACCTCTGGGCTCCTGG (SEQ ID NO: 123).

In an embodiment, the first gRNA of the gRNA pair has the target sequence which comprises or consists of the sequence ATTTCAGGTAAGCCGAGGTT (SEQ ID NO: 207).

In an embodiment, the first gRNA of the gRNA pair has the target sequence which comprises or consists of the sequence TCTTAATAATGTTTCACTGT (SEQ ID NO: 208).

In an embodiment, the second gRNA of the gRNA pair has the target sequence which comprises or consists of the sequence ATAGTTTAAAGGCCAAACCT (SEQ ID NO: 211).

In an embodiment, the second gRNA of the gRNA pair has the target sequence which comprises or consists of the sequence ATAATTTCTATTATATTACA (SEQ ID NO:209).

In an embodiment, the second gRNA of the gRNA pair has the target sequence which comprises or consists of the sequence TTTCATTCATATCAAGAAGA (SEQ ID NO: 210).

In an embodiment, the gRNA pair consists of a first gRNA having a first target sequence which is ATTTCAGGTAAGCCGAGGTT (SEQ ID NO: 207) and a second gRNA having a second target sequence which is ATAATTTCTATTATATTACA (SEQ ID NO:209).

In an embodiment, the gRNA pair consists of a first gRNA having a first target sequence which is ATTTCAGGTAAGCCGAGGTT (SEQ ID NO: 207) and a second gRNA having a second target sequence which is TTTCATTCATATCAAGAAGA (SEQ ID NO: 210).

In an embodiment, the gRNA pair consists of the gRNA pair consists of a first gRNA having a first target sequence which is TCTTAATAATGTTTCACTGT (SEQ ID NO: 208) and a second gRNA having a second target sequence which is TTTCATTCATATCAAGAAGA (SEQ ID NO: 210).

In an embodiment, the gRNA pair consists of the gRNA pair consists of a first gRNA having a first target sequence which is ATAATTTCTATTATATTACA (SEQ ID NO: 209) and a second gRNA having a second target sequence which is ATAGTTTAAAGGCCAAACCT (SEQ ID NO: 211).

In an embodiment, the gRNA pair consists of the gRNA pair consists of a first gRNA having a first target sequence which is TTTCATTCATATCAAGAAGA (SEQ ID NO: 210) and a second gRNA having a second target sequence which is ATAGTTTAAAGGCCAAACCT (SEQ ID NO: 211).

In an embodiment, the gRNA pair consists of the gRNA pair consists of a first gRNA having a first target sequence which is TCTTAATAATGTTTCACTGT (SEQ ID NO: 208) and a second gRNA having a second target sequence which is ATAATTTCTATTATATTACA (SEQ ID NO: 209).

Also provided is a nucleic acid comprising one or more sequences encoding one or both members of a gRNA pair described herein. In an embodiment, the nucleic acid further comprises a sequence encoding a CRISPR nuclease.

Also provided is a nucleic acid comprising a modified dystrophin gene comprising ligated first and second exon ends or intron ends as described herein. In an embodiment, the nucleic acid comprises a hybrid exon sequence set forth in FIG. 5a, 9a(ii) or 17. In an embodiment, the nucleic acid comprises a polynucleotide sequence encoding a hybrid spectrin-like repeat (SLR) having a polypeptide sequence set for in FIG. 16b.

In embodiments, the modified dystrophin gene comprises ligated first and second exon ends defined by the cut sites defined in Table 3 or 6. In embodiments, the modified dystrophin gene comprises ligated first and second intron ends defined by the cut sites defined in Table 8. In a further embodiment, the first cut site is between nucleotides 6769 and 6770 of the DYS gene and the second cut site is between nucleotides 8554 and 8555 of the DYS gene. In a further embodiment, the first cut site is between nucleotides 6833 and 6834 of the DYS gene and the second cut site is between nucleotides 8657 and 8658 of the DYS gene. In a further embodiment, the first cut site is between nucleotides 7228 and 7229 of the DYS gene and the second cut site is between nucleotides 7912 and 7913 of the DYS gene.

Also provided is a modified dystrophin polypeptide encoded by the above-noted nucleic acid.

Also provided is a vector, comprising a nucleic acid described herein. In an embodiment, the vector is a viral vector (e.g. an AAV or a Sendai virus derived vector).

Also provided is a cell (e.g. a host cell,) comprising one or both members of a gRNA pair, nucleic acid, polypeptide and/or vector described herein. In embodiments, the host cell may be prokaryotic or eukaryotic. In an embodiment, the cell is a mammalian cell, in a further embodiment, a human cell. In an embodiment, the cell is a muscle cell (e.g. myoblast or myocyte). In an embodiment, the cell is a cell from a subject suffering from muscular dystrophy (e.g., DMD).

Also provided is a composition, comprising one or both members of a gRNA pair, nucleic acid, polypeptide, vector, and/or cell described herein. In an embodiment, the composition further comprises a CRISPR nuclease or a nucleic acid encoding a CRISPR nuclease. In an embodiment, the composition further comprises a biologically or pharmaceutically acceptable carrier. In an embodiment, the composition is for a use described herein.

Also provided is a kit, comprising one or both members of a gRNA pair, nucleic acid, polypeptide, vector, cell, composition, CRISPR nuclease and/or a nucleic acid encoding a CRISPR nuclease, described herein. In an embodiment, the kit further comprises instructions for performing a method described herein, or is for a use described herein.

In an embodiment, the kit is for use in treating muscular dystrophy in a subject in need thereof.

Also provided is a method for treating muscular dystrophy in a subject, comprising modifying a dystrophin gene and restoring the correct reading frame for dystrophin expression within a cell of said subject according to a method described herein.

Also provided is a method for treating muscular dystrophy in a subject, comprising contacting a cell of the subject with (i)(a) a gRNA pair described herein or one or more nucleic acids encoding said gRNA pair and (b) a CRISPR nuclease polypeptide or a nucleic acid encoding a CRISPR nuclease polypeptide or (ii) a composition described herein.

Also provided is a use of (i)(a) a gRNA pair described herein or one or more nucleic acids encoding said gRNA pair and (b) a CRISPR nuclease polypeptide or a nucleic acid encoding a CRISPR nuclease polypeptide or (ii) a composition described herein, for treating muscular dystrophy in a subject.

Also provided is a use of (i)(a) a gRNA pair described herein or one or more nucleic acids encoding said gRNA pair and (b) a CRISPR nuclease polypeptide or a nucleic acid encoding a CRISPR nuclease polypeptide or (ii) a composition described herein, for the preparation of a medicament for treating muscular dystrophy in a subject.

Also provided is (i)(a) a gRNA pair described herein or one or more nucleic acids encoding said gRNA pair and (b) a CRISPR nuclease polypeptide or a nucleic acid encoding a CRISPR nuclease polypeptide or (ii) a composition described herein, for use in treating muscular dystrophy in a subject.

Also provided is (i)(a) a gRNA pair described herein or one or more nucleic acids encoding said gRNA pair and (b) a CRISPR nuclease polypeptide or a nucleic acid encoding a CRISPR nuclease polypeptide or (ii) a composition described herein, for use in the preparation of a medicament for treating muscular dystrophy in a subject.

In an embodiment, the muscular dystrophy is Duchenne muscular dystrophy.

Also provided is a reaction mixture comprising (a) a gRNA pair described herein or one or more nucleic acids encoding said gRNA pair and (b) a CRISPR nuclease polypeptide or a nucleic acid encoding a CRISPR nuclease polypeptide.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 shows a plasmid used in this study and protospacer adjacent motif (PAM) sites. (a) The expression vector pSpCas(BB)-2A-GFP contains 2 Bbsl sites for the insertion of the protospacer sequence. The guide RNA is under the control of the U6 promoter. Guide RNAs were designed following the identification of PAMs (i.e., NGG sequence) in exons 50 (b) and 54(c) of the DYS gene. The FIG. illustrates the sequence of exons 50 (b) and 54 (c) of the human DYS gene. For exon 50, 10 different PAMs (numbered 1 to 10) were identified; six are in the sense strand and 4 in the antisense strand. For exon 54, 14 PAMs were identified, 5 in the sense strand and 9 in the antisense strand. The GG's of the PAM are shaded in the sense (upper) and antisense (lower) strands. The third nucleotide of the PAMs (i.e. adjacent to the GG's) is also shaded in both strands. See Tables 3, 6 and 8 for exemplary gRNAs targeting sequences adjoining these PAMs;

FIG. 2 shows Transfection efficiency of constructs prepared in accordance with an embodiment of the present invention. The eGFP expression was monitored in 293T (a) and in DMD myoblasts (b and c) after transfection of the pSpCas(BB)-2A-GFP with Lipofectamine 2000. Transfection efficiency was increased in DMD myoblasts following a modification of the transfection protocol with Lipofectamine 2000 (c vs b);

FIG. 3 shows a Surveyor assay for gRNA screening in 293T cells and in myoblasts. The assay was performed on genomic DNA extracted from 293T cells (a and b) or myoblasts (c and d) transfected individually with different gRNAs targeting exons 50 and 54. Screening was performed separately for exon 50 (a and c) and exon 54 (b and d). Genomic DNA of non-transfected cells was used for negative control (NC) for the Surveyor assay. The gRNA numbers correspond with the targeted sequences (Table 1). MW: molecular weight marker;

FIG. 4 shows that the CinDel approach can generate four possible DYS gene modifications. (a) Double-strand breaks created by the Cas9 and different gRNA pairs can theoretically modify the DYS gene four different ways: 1) in light grey (shaded cells of columns 1 and 5), correct junction of the normal codons of exons 50 and 54; 2) in darker grey (shaded cells of columns 2-4, 6-9, 11, 13 and 14, and shaded cells in rows 3 and 4 of columns 10 and 12) the junction of the nucleotides of exons 50 and 54 generates the codon for a new amino acid at the junction site but the remaining codons of exon 54 are normal; 3) in white (non-shaded cells), junction of the nucleotides of exons 50 and 54 results in an incorrect reading frame that changes the remaining codons of exon 54; and 4) in black (dark shaded cells in row 2 of columns 10 and 12), the junction of the nucleotides of exons 50 and 54 generates a new stop codon at the junction site. (b) Different gRNA combinations were experimentally tested in 293T cells and in myoblasts and PCR amplification generated amplicons of the expected sizes. The sequencing of the amplicons of these hybrid exons showed the expected modifications (first row corresponds to “light grey” above; second row corresponds to “darker grey” above; third row corresponds to “white” above; fourth row corresponds to “black” above). MW: molecular weight markers;

FIG. 5 shows that gRNA pairs can induce deletions that restore the reading frame in the DYS gene in DMD myoblasts. Sequence (a) obtained from the amplification of the hybrid exon 50-54 following transfection of the gRNA2-50 and gRNA2-54 pair shows a newly formed codon TAT (coding for tyrosine) at the junction site. This new codon is formed by the nucleotide T from the remaining exon 50 and nucleotides AT from the remaining exon 54. Other in-frame and out-of-frame sequences were also found (b);

FIG. 6 shows that CinDel correction is effective in vivo in the hDMDImdx mouse model. The Tibialis anterior (TA) of hDMD/mdx mice was electroporated with 2 plasmids coding for gRNA2-50 and gRNA2-54. The mice were sacrificed 7 days later. Surveyor assay (a) was performed on amplicons of exons 50 and 54. Two additional bands due to the cutting by the Surveyor enzyme were observed for amplicons of the muscles electroporated with the gRNAs but not in the control muscles (CTL) not electroporated with gRNAs. PCR amplifications (b) of exon 50, exon 54 and hybrid exon 50-54 from DNA extracted from hDMD/mdx muscles electroporated with the gRNA pair. MW: molecular weight markers;

FIG. 7 shows that CinDel correction in myoblasts restored the DYS protein expression in myotubes. (a) Normal wild-type myoblasts (CTL+), uncorrected DMD myoblasts with a deletion of exons 51-53 (CTL−) as well as CinDel-corrected DMD myoblasts (CinDel) were allowed to fuse to form abundant myotubes containing multiple nuclei. Proteins were extracted from these three types of myotubes. The DMD myoblasts (A51-53) were genetically corrected with (b) gRNA2-50 and gRNA2-54 and (c) with gRNA1-50 and gRNA5-54. In b and c, western blot detected no DYS protein in uncorrected DMD myotubes (CTL−), a 427 kDa DYS protein was detected in the wild-type myotubes (CTL+), and a truncated DYS protein (about 400 kDa) was detected in the CinDel-corrected DMD myotubes (CinDel);

FIG. 8 shows the in vivo and in vitro activity of the ribonucleoprotein-nucleic acid complex CRISPRISpCas9. (A) In vitro cutting of the dystrophin exon 54 amplicon by the crRNA (5′ CAGAGAATATCAATGCCTCTGUUUUAGAGCUAUGCUGUUUUG), tracrRNA (Edit-R tracrRNA, Dharmacon) and SpCas9 protein complex. (B) Surveyor assay on amplicon of DMD exon 54 obtained following the transduction in Hela cells with the crRNA:tracrRNA or sgRNA 5-54 targeting exon 54 complexed with SpCas9 protein and delivered with Feldan Shuttle FSA (HHHHHHKWKLFKKIGAVLKVLTTGYARAAARQARA, SEQ ID NO: 196) The presence of expected additional bands confirms that exon 54 amplicons contained INDELs that have been introduced by DSBs in exon 54 by the complex. (C) Delivery of the SpCas9 protein and a pair of sgRNAs in hDMD mouse muscles using different transduction techniques (electroporation, Lipofectamine/RNAiMax™ and Feldan shuttles A (SEQ ID NO: 196) and B (HHHHHHKWKLFKKIGAVLKVLTTGYARAAARQARAHHHHHH, SEQ ID NO: 197). The muscles were longitudinally separated in 4 fragments for each transduction method and the result of each fragment is illustrated. The PCR amplification of the hybrid exon 50-54 produced a band at about 600 bp;

FIG. 9 shows the polynucleotide sequence of hybrid exon 50-54 obtained in vivo following intramuscular delivery of two sgRNA:SpCas9 complexes targeting exons 50 and 54 using the Feldan Shuttle B. (A)(i) Expected (SEQ ID NO: 199) and (ii) obtained (SEQ ID NO: 200) sequences of the hybrid exon 50-54 formed by genome editing using two ribonucleic complexes sgRNA:SpCas9 (gRNAs 1-50 and 5-54) in hDMD/mdx mouse model. In this FIG., forward and reverse primers used for the PCR amplification are highlighted in grey. The remaining section of the exon 50 following the cut by Cas9 is in bold and italic. The junction site is underlined and the remaining part of exon 54 is in bold. (B) Analysis of the homology between the expected sequence (Query) and the sequence obtained (Sbjct) indicate a perfect match between the two sequences in 60% of the clones. This confirms the formation of the hybrid exon 50-54;

FIG. 10 shows a summary of the CinDel therapeutic approach according to embodiments of the present invention. The DYS gene of a DMD patient has a deletion of exons 51, 52 and 53 compared to the wild-type dystrophin. This produces a reading frame shift when the DNA is translated into mRNA resulting into a stop codon in exon 54 which aborts transcription. When exons 50 and 54 are cut by the CinDel treatment, a hybrid exon 50/54 is formed and the reading frame is restored, allowing the normal transcription of the mRNA;

FIG. 11 shows a plasmid used in this study and protospacer adjacent motif (PAM) sites of exemplary gRNAs shown in Tables 6 and 8. (a) The plasmid pX601-AAV-CMV::NLS-SaCas9-NLS-3xHA-bGHpA;U6::Bsal-sgRNA (SaCas9/CRISPR system, Addgene plasmid #61591; SEQ ID NO: 167) containing two Bsal restriction sites necessary for insertion of a protospacer (see below) under the control of the U6 promoter was used in our study. The pX601 plasmid also contains the Cas9 of S. aureus (SaCas9). The pSpCas(BB)-2A-Puro plasmid containing the Cas9 of S. pyogenes was alternatively used for gRNAs designed for SpCas9 (see example 19 for details). Guide RNAs were designed following the identification of PAMs of the S. aureus Cas9 (i.e., NNGRRT or NNGRR(N), see (b) to (h)) or the Streptococcus pyogenes Cas9 (NGG, see (i) and (j)). Panels (b) to (j) show the nucleic acid sequences of f (b) exon 46 (position: 1407236-1407383 in ENS00000198947), (c) exon 47 (position: 1409718-1409867 in ENS00000198947), (d) exon 49 (position: 1502665-1502766 in ENS00000198947), (e) exon 51 (position: 1565292-1565524 in ENS00000198947), (f) exon 52 (position: 1609736-1609853 in ENS0000019894), (g) exon 53 (position: 1659898-1660109 in ENS00000198947), (h) exon 58 (position: 1860391-1860501 in ENS00000198947); (i) part of intron 22; and (j) part of intron 23 of the human DYS gene. (j). The sequences targeted by the gRNA are in bold and the corresponding PAMs are underlined. For exon 46, 2 PAMs (numbered 1 and 2) were identified, 1 in the sense strand and 1 in the antisense strand. For exon 47, 3 PAMs (numbered 3 to 5) were identified, 1 in the sense strand and 2 in the antisense strand. For exon 49, 1 PAM (numbered 6) was identified in the antisense strand. For exon 51, 2 PAMs (numbered 7 and 8) were identified in the antisense strand. For exon 52, 2 PAMs (numbered 9 and 10) were identified, 1 in the sense strand and 1 in the antisense strand. For exon 53, 5 PAMs (numbered 11 to 15) were identified, 3 in the sense strand and 2 in the antisense strand. For exon 58, 3 PAMs (numbered 16 to 18) were identified, 1 in the sense strand and 2 in the antisense strand. For intron 22 (SEQ ID NO: 228), 4 PAMs (numbered 19, 21, 22 and 23) were selected, 2 in the sense strand and 2 in the antisense strand. For intron 23 (SEQ ID NO: 229), 4 PAMs were selected (numbered 20, 24, 25 and 26), 2 in the sense strand and 2 in the antisense strand. The portion of exon 23 downstream of intron 22 and upstream of intron 23 is highlighted in grey. See Tables 6 and 8 for gRNAs targeting sequences adjoining these PAMs;

FIG. 12 shows Surveyor assays for gRNA's screening (SaCas9/CRISPR system) in 293T cells. The assay was performed on genomic DNA extracted from 293T cells (a to g) transfected individually with different gRNAs. Screening was performed separately for exon 46 (a), exon 47 (b), exon 49 (c), exon 51 (d), exon 52 (e), exon 53 (f), exon 58 (g). Genomic DNA of non-transfected cells was used as a control (Ct) for the Surveyor assay. The gRNA numbers correspond to the targeted sequences (Table 6). MW: molecular weight marker. Among the gRNAs we tested, gRNAs 9,11 and 15 show no detectable activity under the conditions tested while gRNAs 1, 2, 3, 4, 5, 6, 7, 8, 10, 12, 13, 14, 16, 17 and 18 exhibited good efficiency as demonstrated by the surveyor enzyme assay;

FIG. 13 shows the activity of different gRNA combinations (SaCas9/CRISPR system) in 293T cells for which PCR amplification generated amplicons of the expected sizes. (a) The combination of gRNAs 1 and 7 and the combination of gRNAs 1 and 8 generated a hybrid exon 46-51. (b) The combination of gRNAs 1 and 12, combination of gRNAs 1 and 13, combination of gRNAs 2 and 14, and the combination of gRNAs 2 and 15 generated the hybrid exon 46-53. (c) A hybrid exon 47-52 can be generated by the combination of gRNAs 5 and 9. (d) A hybrid exon 49-52 can be generated by the combination of gRNAs 6 and 10. (e) A hybrid exon 49-53 can be generated by the combination of gRNAs 6 and 11. The combination of gRNA 3 and 16, combination of gRNA 4 and 17, and the combination of gRNAs 5 and 18 can generate a hybrid exon 47-58;

FIG. 14 shows structural representations of integral spectrin-like repeat R19 and of various hybrid spectrin-like repeats. (a) Primary structure alignments for spectrin-like repeats R19, R20 and R21. Exons associated with these spectrin repeats are identified in gray (below the sequences). The secondary structure for spectrin repeats is represented above the sequences, H for alpha helices and C for the loop segments. Residues between pairs of arrows of the same color are deleted in the resulting hybrid spectrin-like repeats R19-R21. For a patient with a deletion of exons 51-53, the reading frame may be restored by skipping exon 50, thus linking directly exon 49-54. Linking points of deletion of exons 49-54 are highlighted in red. The hybrid exons 2-50/2-54 linking points are highlighted blue and those of hybrid exons 1-50/4-54 in green. (b) Homology models for integral spectrin repeat R19 was obtained from eDystrophin Website. (c) The homology model for the deletion of exons 50-53 (obtained by skipping of exon 50 in a patient with a deletion of exons 51-53). The homology models for (d) hybrid exon 2-50/2-54 and (e) hybrid exon 1-50/4-54 are also illustrated. Structural motifs, as identified in the primary sequence alignment, are colored as follows: helix A is in green, helix B is in orange, and helix C is in blue. Loops AB and BC are in light gray. Colors are darker for spectrin repeat R19 and lighter for spectrin repeat R21;

FIG. 15 shows the activity of different gRNA combinations used in combination with SaCas9. These combinations were experimentally tested in three different myoblast cells from DMD patient with different out-of-frame deletions (delta 49-50, delta 51-53 and delta 51-56) and for which PCR amplification generated amplicons of the expected sizes. The combination of sgRNAs 3 and 16 and the combination of sgRNAs 5 and 18 generated a hybrid exon 47-58 that we amplified by PCR using the forward primer Sense 47′ (5′-CAATAGAAGCAAAGACAAGGTAGTTG) (SEQ ID NO: 194) and the reverse primer Antisense 58′ (5′-GCACAAACTGATTTATGCATGGTAG) (SEQ ID NO: 195);

FIG. 16 shows the localization of the cutting site of 18 exemplary sgRNAs (SaCas9/CRISPR) identified in spectrin like repeats and tested (A) and hybrid spectrin-like repeats 17-19, 17-20, 18-20, 18-21, 19-20 and 18-23 generated using various combinations of gRNAs (B) and (C). The hybrid spectrin like repeats 17-19 was generated from the combination of gRNAs 1 [TTCTCCAGGCTAGAAGAACAA] (SEQ ID NO: 106) and 7 [TTGTGTCACCAGAGTAACAGT] (SEQ ID NO: 112) and from combination of gRNAs 1 [TTCTCCAGGCTAGAAGAACAA] (SEQ ID NO: 106) and 8 [AGTAACCACAGGTTGTGTCAC] (SEQ ID NO: 113). The hybrid spectrin like repeats 17-20 was generated from the combination of sgRNAs 1 [TTCTCCAGGCTAGAAGAACAA] (SEQ ID NO: 106) and 12 [CTTCAGAACCGGAGGCAACAG] (SEQ ID NO: 117) and from the combination of gRNAs 1 [TTCTCCAGGCTAGAAGAACAA] (SEQ ID NO: 106) and 13 [CAACAGTTGAATGAAATGTTA] (SEQ ID NO: 118). The hybrid spectrin like repeats 18-20 was generated from the combination of sgRNAs 5 [CTTATGGGAGCACTTACAAGC] (SEQ ID NO: 110) and 9 [TTCAAATTTTGGGCAGCGGTA] (SEQ ID NO: 114). The hybrid spectrin like repeats 18-21 was generated from the combination of sgRNAs 2 [CTGCTCTTTTCCAGGTTCAAG] (SEQ ID NO: 107) and 14 [GCCAAGCTTGAGTCATGGAAG] (SEQ ID NO: 119) and from the combination of gRNAs 2 [CTGCTCTTTTCCAGGTTCAAG] (SEQ ID NO: 107) and 15 [CTTGGTTTCTGTGATTTTCTT] (SEQ ID NO: 120). The hybrid spectrin like repeats 19-20 was generated from the combinations of sgRNAs 6 [TTGCTTCATTACCTTCACTGG] (SEQ ID NO: 111) and 10 [CAAGAGGCTAGAACAATCATT] (SEQ ID NO: 115) and from the combination of gRNAs 6 [TTGCTTCATTACCTTCACTGG] (SEQ ID NO: 111) and 11 [TTGTACTTCATCCCACTGATT] (SEQ ID NO: 116). The hybrid spectrin-like repeats 18-23 was generated from combination of gRNAs 3 [GTCTGTTTCAGTTACTGGTGG] (SEQ ID NO: 108) and 16 [TCATTTCACAGGCCTTCAAGA] (SEQ ID NO: 121) and 5 [CTTATGGGAGCACTTACAAGC] (SEQ ID NO: 110) and 18 [CAATTACCTCTGGGCTCCTGG] (SEQ ID NO: 123). (A) Arrows indicate cut sites which may be induced by gRNAs. (B) Arrows indicate the hybrid junctions which can be obtained with the listed combinations of sgRNAs;

FIG. 17 shows the DNA sequences of eight hybrid exons obtained from different combinations of gRNAs for use with the SaCas9/CRISPR system. In light grey is represented the first part of the hybrid exon corresponding to the exon (upstream exon) targeted by the first gRNA while in bold is represented the last part of the hybrid exon corresponding to the exon targeted by the second gRNA. The slash (/) indicates the location of the junction;

FIG. 18 illustrates the results of the sequencing of the hybrid exons generated from several gRNAs combinations (SaCas9/CRISPR system) following cloning of PCR product into pMiniT plasmid vector. (a) overall number of clones presenting the precise nucleotide sequences of the expected hybrid exons (identified in FIG. 17) in comparison to the overall number of sequenced clones obtained in 293T cells. (b) shows the sequencing results of hybrid exons 47-58 generated from the combination of gRNAs 3 and 16 and from the combination of gRNAs 5 and 18 in myoblasts from three different DMD patients harbouring a deletion of exons 49-50, or a deletion of exons 51 to 53, or a deletion of exons 51 to 56. Using the combination of gRNAs 3 and 16, we restored a correct reading frame in 70% ( 7/10), 40% ( 4/10) and 88.8% ( 8/9) of the generated hybrid exons in myoblasts harbouring the deletion 49-50, 51-53 and 51-56 respectively. Using the combination of gRNAs 5 and 18, we restored a correct reading frame in 70% ( 7/10), 50% ( 5/10) and 63.6% ( 7/11) of the generated hybrid exons in myoblasts harbouring the deletion 49-50, 51-53 and 51-56 respectively;

FIG. 19 shows the cDNA sequence (SEQ ID NO: 1) of the human DYS gene and the encoded amino acid sequence (SEQ ID NO: 2) of human dystrophin (transcript DMD-001 (ENST00000357033.8) of ENSG00000198947). Exons are shown in the first line via alternating upper and lower case sequence regions;

FIG. 20 shows the cDNA sequence of the human DYS gene (transcript DMD-001 (ENST00000357033.8) of ENSG00000198947). cDNA sequence (SEQ ID NO: 1) is shown in uppercase, grouped by exons. Flanking intronic sequences (25 bases on either side of a given exon) are shown in lowercase, not bold. 25 nts of 5′ UTR are shown in lowercase bold at beginning; 25 nts of 3′ UTR are shown in lowercase bold at end. 25 nts of 5′ UTR+cDNA sequence of exon 1+25 nts of intron sequence at 3′ correspond to SEQ ID NO: 3; cDNA sequences of exons 2 to 78 with flanking 25 nts of intron sequences on each side (5′ and 3′ correspond to SEQ ID NOs: 4-80, respectively; 25 nts of intron sequence at 3′+cDNA sequence of exon 79+25 nts of 3′ UTR correspond to SEQ ID NO: 81;

FIG. 21 shows different gRNA combinations (SaCas9/CRISPR system) that were experimentally tested in vivo in the mouse model hDMD/mdx using two AAV9 vectors. (a) shows that the intravenous (IV) or the intraperitoneal (IP) injections of a combination of 7,5.10¹¹ vg of AAV9 encoding the SaCas9 along with 7,5.10¹¹ vg of AAV9 encoding the gRNA 3 and the gRNA 16 or the gRNA 5 and the gRNA 18 permit to generate the hybrid exons 47-58 in vivo in the heart of a mouse model as demonstrated by the generations of PCR amplicons of expected sizes. (b) shows that intravenous (IV) or intraperitoneal (IP) injections of a combination of 7,5.10¹¹ vg of AAV9 encoding the SaCas9 along with 7,5.10¹¹ vg of AAV9 encoding gRNA 3 and gRNA 16 or gRNA 5 and gRNA 18 permit to generate hybrid exons 47-58 in vivo in the diaphragm of a mouse model as demonstrated by the generation of PCR amplicons of expected sizes. (c) shows that intravenous (IV) injections of a combination of 7,5.10^{11v}g of AAV9 encoding the SaCas9 along with 7,5.10^{11v}g of AAV9 encoding gRNA 3 and gRNA 16 permits to generate hybrid exons 47-58 in vivo in the Tibialis anterior of a mouse model as demonstrated by the generation PCR amplicons of expected size. However, we were not able to detect the hybrid exon in the Tibialis anterior through intraperitoneal injections and for the intravenous injection of AAV9 encoding the gRNA 5 and the gRNA 18;

FIG. 22 shows the activity of gRNAs targeting intron 22 or intron 23 in C2C12 myoblasts. (a) shows a surveyor enzyme assay of the 8 gRNAs selected that target the surrounding introns of the exon 23. gRNA 19, gRNA 21, gRNA 22 and gRNA 23 target intron 22 while gRNA 20, gRNA 24, gRNA 25 and gRNA 26 target intron 23. Target sequences for these gRNAs are shown in Table 8. Genomic DNA of non-transfected and non-selected C2C12 cells was used as control (NT) for the Surveyor assay. (b) Shows the different gRNA combinations (SaCas9/CRISPR system) that were experimentally tested and for which PCR amplification generated two products: the upper PCR amplification product corresponds to wild-type genomic DNA while the second amplification product of lower size corresponds to the deletion of the exon 23;

FIG. 23 shows immunohistochemistry analysis of muscle cuts labelled with an anti-dystrophin antibody in 4 months old mice following intramuscular injection of SpCas9/gRNAs complexed with FSA, FSB and FSC. We tested the intramuscular injection of the combination of the gRNA 20 and 22 and the combination of gRNAs 20 and 23 along with the SpCas9 nuclease complexed with the FSA (SEQ ID NO: 196), FSB (SEQ ID NO: 197) and FSC (HHHHHHLLKLWSRLLKLWTQGRRLKAKRAKAHHHHHH, SEQ ID NO: 198). Three weeks after the intramuscular injection, mice were sacrificed. Following Tibialis anterior harvesting, samples were submitted to the staining of the dystrophin using the primary mouse NCL-Dys2 anti-dystrophin antibody and the secondary Alexa Fluor 546 goat anti-mouse antibody (H+L). (a) shows the expression of dystrophin in a non-treated mouse. Thus, dystrophin positive fibers identify dystrophin revertant fibers. (b) shows the expression of the dystrophin protein in mouse injected with the FSA complexed with the SpCas9 and the combination of gRNAs 20 and 22. (c) shows the expression of the dystrophin protein in mouse injected with the FSB complexed with the SpCas9 and the combination of gRNAs 20 and 22. (d) shows the expression of the dystrophin protein in mouse injected with the FSC complexed with the SpCas9 and the combination of gRNAs 20 and 22. (e) shows the expression of the dystrophin protein in mouse injected with the FSA complexed with the SpCas9 and the combination of gRNAs 20 and 23. (f) shows the expression of the dystrophin protein in mouse injected with the FSB complexed with the SpCas9 and the combination of gRNAs 20 and 23. (g) shows the expression of the dystrophin protein in mouse injected with the FSC complexed with the SpCas9 and the combination of gRNAs 20 and 23;

FIG. 24 shows the number of positive dystrophin fibers identified by immunohistochemistry following intramuscular injection in Rag/mdx mice of gRNAs and SpCas9 complexed with various polypeptide-based shuttles (Feldan Shuttles). FSA, FSB and FSC Feldan Shuttles were used for the delivery of the SpCas9 protein complexed with a combination of gRNAs 22 and 20 or with a combination of gRNAs 23 and 20. In the control muscle (NT) we reported near 37 dystrophin positive fibers (SD+/−4.68), which correspond to revertant fibers. For the combination of gRNAs 4 and 2, we counted 83 (SD+/−18.8), 49.5 (SD+−/8.6) and 186 (SD+/−52) dystrophin positive fibers with Feldan Shuttles FSA, FSB and FSC respectively. For the combination of gRNAs 5 and 2, we counted 39 (SD+/−16.0), 99 (SD+−/15.6) and 199 (SD+/−37.2) dystrophin positive fibers with Feldan Shuttles FSA, FSB and FSC respectively;

FIG. 25 shows the results of the PCR amplification for the detection of the hybrid intron (i.e., deletion of the exon 23) from genomic DNA of Rag/mdx mice following intramuscular injection of gRNAs and SpCas9 complexed with various polypeptide-based shuttles (Feldan Shuttles). (a) shows the amplicons generated from the first PCR using the primers Fw1-i22 and Rev1-i23 along with the poison primer Fw-P. The amplicons detected correspond to the amplification from Fw-P and Rev1-i22 of the wild type genomic DNA, where no deletion of the exon 23 is detected. (b) shows the results of the Nested-PCR amplifications performed on 1 μL of the previous PCR reaction with internal primers Fw2-i22 and Rev2-i22. NT only exhibited amplification of a wild type sequence, as expected. The delivery of the combination of gRNAs 22 and 20 complexed with the Cas9 and the FSA, and FSB, and FSC, allowed the amplification of a truncated PCR product of expected size for deletion of exon 23. Besides, delivery of the combination of gRNAs 23 and 20 complexed with SpCas9 and the FSA, FSB or FSC, allowed the amplification of a truncated PCR product of expected size for deletion of the exon 23. However, the combination of gRNAs 23 and 20 along with the FSA shuttle did not allow the detection of a truncated PCR product. Sequencing of the truncated PCR products are underway to confirm the deletion of the exon 23;

FIG. 26 shows immunohistochemistry analysis of muscle cuts labelled with an anti-dystrophin antibody in 6 weeks old mice following intramuscular injection of SpCas9/gRNAs complexed with FSC or not. In this experiment, we tested the intramuscular injection of the combination of gRNAs 20 and 22 and the combination of gRNAs 20 and 23 with the protein SpCas9 complexed, or not, with the FSC. 6 weeks old mice were injected and Tibialis anterior were collected 3 weeks after the injection. Samples were submitted to the staining of the dystrophin using the primary mouse NCL-Dys2 anti-dystrophin antibody and the secondary Alexa Fluor 546 goat anti-mouse antibody (H+L). (a) and (b) show the expression of the dystrophin protein in muscles injected with gRNAs 20 and 23 complexed with the SpCas9 protein and with no shuttle. (c) shows the expression of the dystrophin protein in muscles injected with gRNAs 20 and 23 complexed with the SpCas9 protein and with the Feldan Shuttle FSC. (d) and (e) show the expression of the dystrophin protein in muscles injected with gRNAs 20 and 22 complexed with the SpCas9 protein and with no shuttle. (f) and (g) show the expression of the dystrophin protein in muscles injected with gRNAs 20 and 22 complexed with the SpCas9 protein and with the Feldan Shuttle FSC;

FIG. 27 shows the number of positive dystrophin fibers identified by immunohistochemistry following intramuscular injection in 6 weeks old Rag/mdx mice of gRNAs and SpCas9 complexed or not with the Feldan Shuttle FSC. Here we compared the delivery of the SpCas9 protein complexed with gRNAs 20 and 22 or gRNAs 20 and 23 supplemented, or not, with the FSC which previously exhibited the most promising results. In muscles injected with and SpCas9 complexed with gRNAs 20 and 22 or gRNAs 20 and 23 without shuttle, we counted 101 (SD+/−6.2) and 120 (SD+/−38.9) dystrophin positive fibers, respectively. When the FSC was used in combination to the SpCas9 and gRNAs 20 and 22 or gRNAs 20 and 23 we detected a higher amount of dystrophin positive fibers as we respectively reported 151 (SD+/−93.3) and 166 (SD+/−25.6) positive fibers; and

FIG. 28 shows the PCR detection of the hybrid intron (i.e., deletion of exon 23) in 6 weeks old mice using the Feldan Shuttle FSC. (a) shows the amplicons generated from the first PCR using the primers Fw1-i22 and Rev1-i23 along with the poison primer Fw-P. The amplicons detected correspond to the amplification from Fw-P and Rev1-i22 of the wild type genomic DNA, where no deletion of the exon 23 is detected. (b) shows the results of the Nested-PCR amplifications performed on 1 μL of the previous PCR reaction with internal primers Fw2-i22 and Rev2-i22. NT only exhibited amplification of a wild type sequence, as expected. The delivery of the combination of gRNAs 22 and 20 complexed with the Cas9 with or without the shuttle FSC, allowed the amplification of a truncated PCR product of expected size for deletion of exon 23. Besides, delivery of the combination of gRNAs 23 and 20 complexed with SpCas9 with or without FSC, allowed the amplification of a truncated PCR product of expected size for deletion of the exon 23 except for the first lane regarding the gRNAs 20 and 23 with the FSC which corresponds to a failed injection. Sequencing of the truncated PCR products are underway to confirm the deletion of the exon 23.

### DESCRIPTION OF ILLUSTRATIVE EMBODIMENTS

In a first aspect, the present invention is based on Applicants' finding that by introducing mutations within exon sequences located up-stream and downstream of an endogenous frameshift or nonsense mutation in the DYS gene of a cell, it is possible to restore the correct reading frame and in turn restore dystrophin expression within the cell. Preferably, the mutations correcting the reading frame are introduced as close as possible to the endogenous frameshift or nonsense mutation, but within an exon. Given that the sites of the engineered mutations are within one or more exons, the corrected gene has a fusion of two exon portions (i.e. which are normally not contiguous with one another), and at the same time restoring the correct reading frame of the DYS gene. Using this approach, Applicants have found that it is possible to restore dystrophin expression within the cell to produce a dystrophin protein having smaller deletions and being functionally closer to the wild-type dystrophin protein.

Several approaches can be used to introduce one or more mutations within one or more exons of the dystrophin gene and restore dystrophin expression. For example, sequence-specific nucleases such as meganucleases, zinc finger nucleases (ZFNs), transcription activator-like effector nucleases (TALENs) and the CRISPR/Cas9 system can be used to introduce one or more targeted mutations within one or more exons of the DYS gene to restore dystrophin expression. Depending on the endogenous mutation already present in DYS gene within the cell, the method of the present invention may or may not lead to the expression of a wild-type dystrophin protein. However, it has been found that by targeting exon sequences (as opposed to introns) which are close to the endogenous mutation(s), the cell will advantageously express a dystrophin protein having a function which is closer to that of the wild-type dystrophin protein.

In a particular embodiment, the present invention uses the CRISPR system to introduce further mutations within exons of a mutated dystrophin gene within a cell. The CRISPR system is a defense mechanism identified in bacterial species [37-42]. It has been modified to allow gene editing in mammalian cells. The modified system still uses a Cas9 nuclease to generate double-strand breaks (DSB) at a specific DNA target sequence [43, 44]. The recognition of the cleavage site is determined by base pairing of the gRNA with the target DNA and the presence of a trinucleotide called PAM (protospacer adjacent motif) juxtaposed to the targeted DNA sequence [45]. This PAM is NGG for the Cas9 of S. pyogenes, the most commonly used enzyme [46, 47]. This PAM is NNGRRT or NNGRR(N) for the high efficiency Cas9 of Staphylococcus aureus, a smaller Cas9 which can advantageously be used in the context of adeno-associated virus delivery and paired nickase (in case of the D10A and N580A variant) applications.

In a further aspect, the present invention is based on Applicant's finding that delivery of a combination of gRNAs and CRISPR nuclease (e.g., Cas9) in cells is particularly effective using polypeptide-based shuttles (Feldan Shuttles, from Feldan Therapeutics).

The shuttles deliver proteins or nucleic acids (e.g., gRNAs) directly inside cells. Once delivered, these proteins/gRNAs dislodge from the carrier and are free to induce genetic modifications. The shuttles are 100% protein-based and are rapidly degraded in the cell, leaving no toxic residues. They are capable of delivering proteins/nucleic acids in multiple cell types without altering cell viability. The Shuttles are virus-free, DNA- and RNA-free, thereby eliminating potential mutagenic risks.

Useful polypeptide-based shuttles and methods of preparing and using polypeptide-based (Feldan) shuttles are described in U.S. application Ser. No. 15/094,365 (Polypeptide-based shuttle agents for improving the transduction efficiency of polypeptide cargos to the cytosol of target eukaryotic cells, uses thereof, methods and kits relating to same), filed on Apr. 8, 2016; in International (PCT) application No. PCT/CA2016/050403 (Polypeptide-based shuttle agents for improving the transduction efficiency of polypeptide cargos to the cytosol of target eukaryotic cells, uses thereof, methods and kits relating to same), filed on Apr. 8, 2016; in U.S. provisional application No. 62/320,065 (Peptide shuttle based gene disruption), filed on Apr. 8, 2016; and in U.S. Provisional Patent Application No. 62/407,232 (Rationally-designed synthetic peptide shuttle agents for delivering polypeptide cargos from an extracellular space to the cytosol and/or nucleus of a target eukaryotic cell, uses thereof, methods and kits relating to same), filed on Oct. 12, 2016, which are incorporated by reference in their entirety.

### Definitions

In order to provide clear and consistent understanding of the terms in the instant application, the following definitions are provided.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

The articles “a,” “an” and “the” are used herein to refer to one or to more than one (i.e., to at least one) of the grammatical object of the article.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps and are used interchangeably with, the phrases “including but not limited to” and “comprising but not limited to”.

For the recitation of numeric ranges herein, each intervening number there between with the same degree of precision is explicitly contemplated. For example, for the range of 18-20, the numbers 18, 19 and 20 are explicitly contemplated, and for the range 6.0-7.0, the number 6.0, 6.1, 6.2, 6.3, 6.4, 6.5, 6.6, 6.7, 6.8, 6.9, and 7.0 are explicitly contemplated. The terms “such as” are used herein to mean, and is used interchangeably with, the phrase “such as but not limited to”.

Practice of the methods, as well as preparation and use of the products and compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

Various genes and nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule, which is expressed using a recombinant nucleic acid construct created by means of molecular biological techniques. The term “recombinant” when made in reference to genetic composition refers to a gamete or progeny or cell or genome with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Coding sequence” or “encoding nucleic acid” as used herein means the nucleic acids (RNA or DNA molecule) that comprise a nucleotide sequence which encodes a protein or gRNA. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. In an embodiment, the subject or patient may suffer from DMA and has a mutated dystrophin gene. The subject or patient may be undergoing other forms of treatment.

“Vector” as used herein means a nucleic acid sequence containing an origin of replication. A “vector” as described herein refers to a vehicle that carries a nucleic acid sequence and serves to introduce the nucleic acid sequence into a host cell. In an embodiment, the vector will comprise transcriptional regulatory sequences or a promoter operably-linked to a nucleic acid comprising a sequence capable of encoding a gRNA, nuclease or polypeptide described herein. In embodiments, the promoter is a U6 or CBh promoter. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since, for example, enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals which induce or control transcription of protein coding sequences with which they are operably-linked. A vector may be a viral vector (e.g., AAV), bacteriophage, bacterial artificial chromosome or yeast artificial chromosome. A vector may be a DNA or RNA vector. A vector may be a self-replicating extrachromosomal vector, and preferably, is a DNA plasmid. For example, the vector may comprise nucleic acid sequence(s) that/which encode(s) at least one gRNA and/or CRISPR nuclease (e.g. Cas9) described herein. Alternatively, the vector may comprise nucleic acid sequence(s) that/which encode(s) one or more CRISPR nucleases (Cas9 or Cpf1) at least one (preferably at least 2) gRNA nucleotide sequence of the present invention. A vector for expressing one or more gRNA will comprise a “DNA” sequence of the gRNA.

“Adeno-associated virus” or “AAV” as used interchangeably herein refers to a small virus belonging to the genus Dependovirus of the Parvoviridae family that infects humans and some other primate species. AAV is not known to cause disease and consequently the virus causes a very mild immune response.

### Sequence Similarity

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “substantially homologous” to another sequence if the two sequences are substantially identical and the functional activity of the sequences is conserved (as used herein, the term “homologous” does not infer evolutionary relatedness, but rather refers to substantial sequence identity, and thus is interchangeable with the terms “identity”/“identical”). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. For the sake of brevity, the units (e.g., 66, 67 . . . 81, 82, . . . 91, 92% . . . ) have not systematically been recited but are considered, nevertheless, within the scope of the present invention.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, at least 95%, at least 96%, at least 98% or at least 99%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman (Pearson and Lipman 1988), and the computerized implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al. (Altschul et al. 1990) 1990 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO4, 7% sodium dodecyl sulfate (SDS), 1 mM EDTA at 65° C., and washing in 0.2×SSC/0.1% SDS at 42° C. (Ausubel 2010). Alternatively, hybridization to filter-bound sequences under stringent conditions may, for example, be performed in 0.5 M NaHPO4, 7% SDS, 1 mM EDTA at 65° C., and washing in 0.1×SSC/0.1% SDS at 680° C. (Ausubel 2010). Hybridization conditions may be modified in accordance with known methods depending on the sequence of interest (Tijssen 1993). Generally, stringent conditions are selected to be about 5° C. lower than the thermal melting point for the specific sequence at a defined ionic strength and pH.

“Binding” refers to a sequence-specific, non-covalent interaction between macromolecules (e.g., between a protein and a nucleic acid or between a gRNA and a target polynucleotide or between a gRNA and a CRISPR nuclease (e.g., Cas9, Cpf1). Not all components of a binding interaction need to be sequence-specific (e.g., contacts with phosphate residues in a DNA backbone), as long as the interaction as a whole is sequence-specific. “Affinity” refers to the strength of binding: increased binding affinity being correlated with a lower Kd. A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

As used herein, a “target gene”, “targeted gene”, “targeted polynucleotide” or “targeted gene sequence” corresponds to the polynucleotide within a cell that will be modified, in an embodiment by the introduction a gRNA pair and a CRISPR nuclease. It corresponds to an endogenous gene naturally present within a cell. In an embodiment, the targeted gene is a DYS gene comprising one or more frameshift or nonsense mutations associated with the development of MD (e.g., DMD or BMD). One or both alleles of a targeted gene may be modified within a cell in accordance with the present invention.

A “frameshift mutation” is a mutation within a polynucleotide sequence (e.g., a DNA gene sequence) caused by indels (insertions or deletions) of a number of nucleotides in a DNA sequence that is not divisible by three. Due to the triplet nature of gene expression by codons, a frameshift mutation caused by one or more insertion or deletion changes the reading frame (the grouping of the codons), resulting in a different translation product (mutated polypeptide) from the original (wild type polypeptide). The frameshift mutation can result in a truncated polypeptide (by generating a premature stop codon), in a larger polypeptide (by removing a stop codon), and/or in a completely or partially different amino acid sequence. Mutated polypeptides caused by one or more frameshift mutations in the wild-type polynucleotide (gene) sequence are often non-functional, such as in the case of mutations in the DYS gene causing MD (e.g., DMD or BMD).

A “nonsense mutation” is a point mutation within a polynucleotide sequence (a DNA gene sequence) that results in a premature stop codon, or a nonsense codon in the transcribed mRNA, and in a truncated, incomplete, and usually nonfunctional protein product. “A nonsense mutation” within the context of the present invention encompasses the presence of at least one nonsense mutation within a polynucleotide (e.g. gene) sequence and thus includes the presence of a plurality of such mutations.

As used herein an “endogenous frameshift mutation” or an “endogenous nonsense mutation” is a mutation which is naturally present within a gene of a subject. For example, in the context of muscular dystrophy (DMD or BMD), an endogenous frameshift mutation or an endogenous nonsense mutation is a mutation found in the dystrophin gene (in one or both alleles) of cells of a subject suffering from muscular dystrophy. The presence of such endogenous mutation (frameshift or nonsense) is responsible for the development of the disease. The presence of such mutation results in a reduced level of dystrophin protein expression, in the production of an unstable protein (with a reduced half-life), in the production of a a truncated protein and/or in an inactive protein (or less active protein compared to the wild-type endogenous protein).

“Promoter” as used herein means a synthetic or naturally-derived nucleic acid molecule which is capable of conferring, modulating or controlling (e.g., activating, enhancing and/or repressing) expression of a nucleic acid in a cell. A promoter may comprise one or more specific transcriptional regulatory sequences to further enhance or repress expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the U6 promoter, bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter. In embodiments, the U6 promotor is used to express one or more gRNAs in a cell.

### CRISPR System

CRISPR technology is a system for genome editing, e.g., for modification of the expression of a specific gene.

This system stems from findings in bacterial and archaea which have developed adaptive immune defenses termed clustered regularly interspaced short palindromic repeats (CRISPR) systems, which use CRISPR targeting RNAs (crRNAs) and Cas proteins to degrade complementary sequences present in invading viral and plasmid DNA. Jinek et al. (47) and Mali et al. (41) have engineered a type II bacterial CRISPR system using custom guide RNA (gRNA) to induce double strand break(s) in DNA. In one system, the Cas9 protein was directed to genomic target sites by a synthetically reconstituted “guide RNA” (“gRNA), which corresponds to a crRNA and tracrRNA which can be used separately or fused together, that obviates the need for RNase III and crRNA processing in general. It comprises a “gRNA guide sequence” or “gRNA target sequence” and an RNA sequence (Cas9 recognition sequence), which is necessary for Cas (e.g., Cas9) binding to the targeted gene. The gRNA guide sequence is the sequence which confers specificity. It hybridizes with (i.e., it is complementary to) the opposite strand of a target sequence (i.e., it corresponds to the RNA sequence of a DNA target sequence). Other CRISPR systems using difference CRISPR nucleases have been developed and are known in the art (e.g., using the Cpf1 nuclease instead of a Cas9 nuclease).

One may alternatively use in accordance with the present invention a pair of specifically designed gRNAs in combination with a Cas9 nickase or in combination with a dCas9-FolkI nuclease to cut both strands of DNA.

In embodiments, provided herein are CRISPR/nuclease-based engineered systems for use in modifying the DYS gene and restoring its correct reading frame. The CRISPR/nuclease-based systems of the present invention include at least one nuclease (e.g. a Cas9 or Cpf1 nuclease) and at least one gRNA targeting the endogenous DYS gene in target cells.

Accordingly, in an aspect, the present invention involves the design and preparation of one or more gRNAs for inducing a DSB (or two single stranded breaks (SSB) in the case of a nickase) in a DYS gene. The gRNAs (targeting the DYS gene) and the nuclease are then used together to introduce the desired modification(s) (i.e., gene-editing events), e.g., by NHEJ or HDR, within the genome of one or more target cells.

The CRISPR/nuclease-based systems of the present invention include at least one CRISPR nuclease (e.g. a Cas9 or Cpf1 nuclease) and at least one gRNA targeting the endogenous DYS gene in target cells. gRNAs

In order to cut DNA at a specific site, CRISPR nucleases require the presence of a gRNA and of a protospacer adjacent motif (PAM) on the targeted gene. The PAM, immediately follows (i.e., is adjacent to) the gRNA target sequence in the targeted polynucleotide gene sequence. The PAM is located at the 3′ end or 5′ end of the sgRNA target sequence (depending on the CRISPR nuclease used) but is not included in the sgRNA guide sequence. For example, the PAM for Cas9 CRSIPR nucleases is located at the 3′ end of the sgRNA target sequence on the target gene while the PAM for Cpf1 nucleases is located at the 5′ end of the sgRNA target sequence on the target gene. Different CRISPR nucleases also require a different PAM. Accordingly, selection of a specific polynucleotide gRNA target sequence (e.g., in the DYS gene nucleic acid sequence) is generally based on the CRISPR nuclease used. The PAM for the Streptococcus pyogenes Cas9 CRISPR system is 5′-NRG-3′, where R is either A or G, and characterizes the specificity of this system in human cells. The PAM of S. aureus Cas9 is NNGRR(T). The S. pyogenes Type II system naturally prefers to use an “NGG” sequence, where “N” can be any nucleotide, but also accepts other PAM sequences, such as “NAG” in engineered systems. Similarly, the Cas9 derived from Neisseria meningitidis (NmCas9) normally has a native PAM of NNNNGATT, but has activity across a variety of PAMs, including a highly degenerate NNNNGNNN PAM. The PAM for a AsCpf1 or LbCpf1 CRISPR nuclease is TTTN. In a preferred embodiment, the PAM for a Cas9 or Cpf1 protein is used in accordance with the present invention is a NGG trinucleotide-sequence (Cas9) or TTTN (AsCpf1 and LbCpf1). Table 1 below provides a list of non-limiting examples of CRISPR/nuclease systems with their respective PAM sequences.

TABLE 1
Non-exhaustive list of CRISPR-nuclease systems from different species
(see. Mohanraju, P. et al. (60); Shmakov, S et al. (61); and Zetsche,
B. et al. (62). Also included are engineered variants recognizing
alternative PAM sequences (see Kleinstiver, B P. et al., (63)).

| CRISPR nuclease | PAM Sequence |
|---|---|
| Streptococcus pyogenes (SP); SpCas9 | NGG + NAG |
| SpCas9 D1135E variant | NGG (reduced NAG binding) |
| SpCas9 VRER variant | NGCG |
| SpCas9 EQR variant | NGAG |
| SpCas9 VQR variant | NGAN or NGNG |
| Staphylococcus aureus (SA); SaCas9 | NNGRRT or NNGRR(N) |
| SaCas9 KKH variant | NNNRRT |
| Neisseria meningitides (NM) | NNNNGATT |
| Streptococcus thermophilus (ST) | NNAGAAW |
| Treponema denticola (TD) | NAAAAC |
| AsCpf1 | TTTN |
| AsCpf1 S542R/K607R | TYCV |
| AsCpf1 S542R/K548V/N552R | TATV |
| LbCpf1 | TTTN |
| LbCpf1 G532R/K595R | TYCV |

As used herein, the expression “gRNA” or “sgRNA” refers to a guide RNA which works in combination with a CRISPR nuclease to introduce a cut into DNA. The sgRNA comprises a sgRNA guide sequence (corresponding to the target sequence) and a “CRISPR nuclease recognition sequence”.

As used herein, the expression “gRNA guide sequence” refers to the corresponding RNA sequence of the “gRNA target sequence” (also known as the spacer sequence). Therefore, it is the RNA sequence equivalent of the protospacer on the target polynucleotide gene sequence. It does not include the corresponding PAM sequence in the genomic DNA. It is the sequence that confers target specificity. In embodiments, the gRNA guide sequence is linked to a CRISPR nuclease recognition sequence which binds to the nuclease (e.g., Cas9/Cpf1). The sgRNA guide sequence recognizes and binds to the targeted gene of interest. It hybridizes with (i.e., is complementary to) the opposite strand of a target gene sequence, which comprises the PAM (i.e., it hybridizes with the DNA strand opposite to the PAM). As noted above, the “PAM” is the nucleic acid sequence, that immediately follows (is contiguous to) the target sequence on the DYS gene or target polynucleotide but is not in the gRNA.

In embodiments, the gRNA is a fusion between the gRNA guide sequence and the CRISPR nuclease recognition sequence (CRISPR repeat and optionally tracrRNA). It provides both targeting specificity and scaffolding/binding ability for the CRSIPR nuclease of the present invention. gRNAs of the present invention do not exist in nature, i.e., they are non-naturally occurring nucleic acid(s).

A “target region”, “target sequence” or “protospacer” in the context of gRNAs and CRISPR system of the present invention are used herein interchangeably and refers to the region of the target gene, which is targeted by the CRISPR/nuclease-based system, without the PAM. It refers to the sequence corresponding to the nucleotides that precede the PAM (i.e., in 5′ or 3′ of the PAM, depending of the CRISPR nuclease) in the genomic DNA. It is the sequence that is included into a gRNA expression construct (e.g., vector/plasmid/AVV). The CRISPR/nuclease-based system may include at least one (i.e., one or more, preferably two) gRNAs, wherein each gRNA targets a different DNA sequence on the target gene. The target DNA sequences may be overlapping. The target sequence or protospacer is followed or preceded by a PAM sequence at an end (3′ or 5′ depending on the CRISPR nuclease used) of the protospacer. Generally, the target sequence is immediately adjacent (i.e., is contiguous) to the PAM sequence (it is located on the 5′ end of the PAM for SpCas9-like nuclease and at the 3′ end for Cpf1-like nuclease).

A “CRISPR nuclease recognition sequence” as used herein refers broadly to one or more RNA sequences (or RNA motifs) required for the binding and/or activity (including activation) of the CRISPR nuclease on the target gene (such as “UAAUUUCUAC UCUUGUAGAU” (SEQ ID NO: 168) in 5′ for Cpf1 nuclease). It encompasses the structural piece (the repeat sequence) that normally complements the tracrRNA and the tracrRNA sequence. Some CRISPR nucleases require longer RNA sequences than other to function. Also, some CRISPR nucleases require multiple RNA sequences (motifs) to function while others only require a single short RNA sequence/motif. For example, Cas9 proteins require a tracrRNA sequence in addition to a crRNA sequence (repeat) to function while Cpf1 only requires a crRNA sequence. Thus, unlike Cas9, which requires both crRNA sequence and a tracrRNA sequence (or a fusion or both crRNA and tracrRNA) to mediate interference, Cpf1 processes crRNA arrays independent of tracrRNA, and Cpf1-crRNA complexes alone cleave target DNA molecules, without the requirement for any additional RNA species (see Zetsche et al., PMID: 26422227).

The “CRISPR nuclease recognition sequence” included in the sgRNA described herein is thus selected based on the specific CRISPR nuclease used. It includes direct repeat sequences and any other RNA sequence known to be necessary for the selected CRISPR nuclease binding and/or activity. Various RNA sequences which can be fused to an RNA guide sequence to enable proper functioning of CRISPR nucleases (referred to herein as CRISPR nuclease recognition sequence) are well known in the art and can be used in accordance with the present invention. The “CRISPR nuclease recognition sequence” may thus include a crRNA sequence only (e.g., for Cpf1 activity, such as the CRISPR nuclease recognition sequence UAAUUUCUAC UCUUGUAGAU set forth in SEQ ID NO: 168) or may include additional sequences (e.g., tracrRNA sequence necessary for Cas9 activity, such as the CRISPR nuclease recognition sequence set forth in SEQ ID NO: 166 which includes both crRNA and tracrRNA sequences). Furthermore, in accordance with the present invention and as well known in the art, RNA motifs necessary for CRISPR nuclease binding and activity may be provided separately (e.g., (i) RNA guide sequence-crRNA CRISPR recognition sequence” (also known as crRNA) in one RNA molecule and (ii) a tracrRNA CRISPR recognition sequence on another, separate RNA molecule. Alternatively, all necessary RNA sequences (motifs) may be fused together in a single RNA guide. The CRISPR recognition sequence is preferably fused directly to the gRNA guide sequence (in 3′ (e.g., Cas9) or 5′ (Cpf1) depending on the CRISPR nuclease used) but may include a spacer sequence separating two RNA motifs.

In embodiments, the CRISPR nuclease recognition sequence is a Cas9 recognition sequence having at least 65 nucleotides. In embodiments, the CRISPR nuclease recognition sequence is a Cas9 CRISPR nuclease recognition sequence having at least 85 nucleotides. In embodiments, the CRISPR nuclease recognition sequence is a Cpf1 recognition sequence (5′ direct repeat) having about 19 nucleotides. In a particular embodiment, the Cas9 recognition sequence comprises (or consists of) the sequence as set forth in SEQ ID NO: 166. In a particular embodiment, the AsCpf1 recognition sequence comprises (or consists of) the sequence UAAUUUCUAC UCUUGUAGAU (SEQ ID NO: 168). The gRNA of the present invention may comprise any variant of the above noted sequences, provided that it allows for the proper functioning of the selected CRISPR nuclease (e.g., binding of the CRISPR nuclease protein to the DYS gene and/or target polynucleotide sequence(s)).

Together, the RNA guide sequence and CRSIPR nuclease recognition sequence(s) provide both targeting specificity and scaffolding/binding ability for the CRSIPR nuclease of the present invention. sgRNAs of the present invention do not exist in nature, i.e., is a non-naturally occurring nucleic acid(s).

In embodiments, the CRISPR nuclease (e.g., Cas9 or Cpf1) recognition sequence is a CRISPR nuclease recognition sequence having at least 65 nucleotides. In embodiments, the CRISPR nuclease recognition sequence is a CRISPR nuclease recognition sequence having at least 85 nucleotides.

As noted above not all CRISPR nucleases require a tracrRNA to function. Cpf1 is a single crRNA-guided endonuclease. Unlike Cas9, which requires both an RNA guide sequence (crRNA) and a tracrRNA (or a fusion or both crRNA and tracrRNA) to mediate interference, Cpf1 processes crRNA arrays independent of tracrRNA, and Cpf1-crRNA complexes alone cleave target DNA molecules, without the requirement for any additional RNA species (see Zetsche et al. (62)).

In embodiments, the gRNA may comprise a “G” at the 5′ end of its polynucleotide sequence. The presence of a “G” in 5′ is preferred when the gRNA is expressed under the control of the U6 promoter (Koo T. et al. (65)). The CRISPR/nuclease system of the present invention may use gRNAs of varying lengths. The gRNA may comprise a gRNA guide sequence of at least 10 nts, at least 11 nts, at least a 12 nts, at least a 13 nts, at least a 14 nts, at least a 15 nts, at least a 16 nts, at least a 17 nts, at least a 18 nts, at least a 19 nts, at least a 20 nts, at least a 21 nts, at least a 22 nts, at least a 23 nts, at least a 24 nts, at least a 25 nts, at least a 30 nts, or at least a 35 nts of a target sequence in the DYS gene (such target sequence is followed or preceded by a PAM in the DYS gene but is not part of the gRNA). In embodiments, the “gRNA guide sequence” or “gRNA target sequence” may be least 10 nucleotides long, preferably 10-40 nts long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nts long), more preferably 17-30 nts long, more preferably 17-22 nucleotides long. In embodiments, the gRNA guide sequence is 10-40, 10-30, 12-30, 15-30, 18-30, or 10-22 nucleotides long. In embodiments, the PAM sequence is “NGG”, where “N” can be any nucleotide. In embodiments, the PAM sequence is NNGRRT or NNGRR(N), where “N” can be any nucleotide and R is A or G. In embodiments, the PAM sequence is “TTTN”, where “N” can be any nucleotide. gRNAs may target any region of a target gene (e.g., DYS) which is immediately adjacent (contiguous, adjoining, in 5′ or 3′) to a PAM (e.g., NGG/TTTN or CCN/NAAA for a PAM that would be located on the opposite strand) sequence. In embodiments, the gRNA of the present invention has a target sequence which is located in an exon (the gRNA guide sequence consists of the RNA sequence of the target (DNA) sequence which is located in an exon). In embodiments, the gRNA of the present invention has a target sequence which is located in an intron (the gRNA guide sequence consists of the RNA sequence of the target (DNA) sequence which is located in an intron). In embodiments, the gRNA may target any region (sequence) which is followed (or preceded, depending on the CRISPR nuclease used) by a PAM in the DYS gene which may be used to restore its correct reading frame.

The number of sgRNAs administered to or expressed in a target cell in accordance with the methods of the present invention may be at least 1 gRNA, preferably at least two gRNAs.

Although a perfect match between the gRNA guide sequence and the DNA sequence on the targeted gene is preferred, a mismatch between a gRNA guide sequence and target sequence on the gene sequence of interest is also permitted as along as it still allows hybridization of the gRNA with the complementary strand of the gRNA target polynucleotide sequence on the targeted gene. A seed sequence of between 8-12 consecutive nucleotides in the gRNA, which perfectly matches a corresponding portion of the gRNA target sequence is preferred for proper recognition of the target sequence. The remainder of the guide sequence may comprise one or more mismatches. In general, gRNA activity is inversely correlated with the number of mismatches. Preferably, the gRNA of the present invention comprises 7 mismatches, 6 mismatches, 5 mismatches, 4 mismatches, 3 mismatches, more preferably 2 mismatches, or less, and even more preferably no mismatch, with the corresponding gRNA target gene sequence (less the PAM). Preferably, the gRNA nucleic acid sequence is at least 90%, 91%, 92%, 93%, 94%, 95%, 96%, 97%, 98% and 99% identical to the gRNA target polynucleotide sequence in the gene of interest (e.g., DYS). Of course, the smaller the number of nucleotides in the gRNA guide sequence the smaller the number of mismatches tolerated. The binding affinity is thought to depend on the sum of matching gRNA-DNA combinations.

Any gRNA guide sequence can be selected in the target gene, as long as it allows introducing at the proper location, the desired modification(s) (e.g., insertions/deletions or selected target modification(s)). Accordingly, the gRNA guide sequence or target sequence of the present invention may be in coding or non-coding regions of the DYS gene (i.e., exons or introns). Of course the complementary strand of the sequence may alternatively and equally be used to identify proper PAM and gRNA target/guide sequences.

### CRISPR Nucleases

Recently, Tsai et al. (64). have designed recombinant dCas9-FoKI dimeric nucleases (RFNs) that can recognize extended sequences and edit endogenous genes with high efficiency in human cells. These nucleases comprise a dimerization-dependent wild type FokI nuclease domain fused to a catalytically inactive Cas9 (dCas9) protein. Dimers of the fusion proteins mediate sequence specific DNA cleavage when bound to target sites composed of two half-sites (each bound to a dCas9 (i.e., a Cas9 nuclease devoid of nuclease activity) monomer domain) with a spacer sequence between them. The dCas9-FoKI dimeric nucleases require dimerization for efficient genome editing activity and thus, use two gRNAs for introducing a cut into DNA.

The recombinant CRISPR nuclease that may be used in accordance with the present invention is i) derived from a naturally occurring nuclease (e.g., Cas or Cpf1 nuclease); and ii) has a nuclease (or nickase) activity to introduce a DSB in cellular DNA when in the presence of appropriate gRNA(s). Thus, as used herein, the term “CRISPR nuclease” refers to a recombinant protein which is derived from a naturally occurring nuclease which has nuclease activity and which functions with the gRNAs of the present invention to introduce DSBs in the targets of interest, e.g., the DYS gene. In embodiments, the CRISPR nuclease is spCas9. In embodiments, the CRISPR nuclease is SaCas9. In embodiments, the CRISPR nuclease is Cpf1. Exemplary CRISPR nucleases that may be used in accordance with the present invention are provided in Table 1 above. A variant of Cas9 can be a Cas9 nuclease that is obtained by protein engineering or by random mutagenesis (i.e., is non-naturally occurring). Such Cas9 variants remain functional and may be obtained by mutations (deletions, insertions and/or substitutions) of the amino acid sequence of a naturally occurring Cas9, such as that of S. pyogenes or S. aureus.

CRISPR nucleases such as Cas9 nucleases cut 3-4 bp upstream of the PAM sequence. CRISPR nucleases such as Cpf1 on the other hand, generate a 5′ overhang. The cut occurs 19 bp after the PAM on the targeted (+) strand and 23 bp on the opposite strand (62). There can be some off-target DSBs using wildtype Cas9. The degree of off-target effects depends on a number of factors, including: how closely homologous the off-target sites are compared to the on-target site, the specific site sequence, and the concentration of nuclease and guide RNA (gRNA). These considerations only matter if the PAM sequence is immediately adjacent to the nearly homologous target sites. The mere presence of additional PAM sequences should not be sufficient to generate off target DSBs; there needs to be extensive homology of the protospacer followed or preceded by PAM.

Optimization of Codon Degeneracy

Because CRISPR nuclease proteins are (or are derived from) proteins normally expressed in bacteria, it may be advantageous to modify their nucleic acid sequences for optimal expression in eukaryotic cells (e.g., mammalian cells) when designing and preparing CRISPR nuclease recombinant proteins.

Accordingly, the following codon chart (Table 2) may be used, in a site-directed mutagenic scheme, to produce nucleic acids encoding the same or slightly different amino acid sequences of a given nucleic acid:

TABLE 2
Codons encoding the same amino acid

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GM GAG |
| Phenylalanine | Phe | F | UUC UUU |
| Glycine | Gly | G | GGA GGC GGG GGU |
| Histidine | His | H | CAC CAU |
| isoleucine | Ile | I | AUA AUC AUU |
| Lysine | Lys | K | AAA MG |
| Leucine | Leu | L | UUA UUG CUA CUC CUG CUU |
| Methionine | Met | M | AUG |
| Asparagine | Asn | N | MC MU |
| Proline | Pro | P | CCA CCC CCG CCU |
| Glutamine | Gln | Q | CM CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

### Dystrophin

As used herein, the term “dystrophin gene” refers to the gene encoding the dystrophin protein (see accessions HGNC:2928, Entrez Gene: 1756; OMIM: 300377; Ensembl: ENSG00000198947; UniProtKB: P11532 and GenBank: NC_000023.11, the contents of which are herein incorporated by reference). The term “dystrophin gene” includes allelic variants of the gene which are found in nature (i.e., within subjects normally expressing the dystrophin protein). The dystrophin gene contains at least eight independent, tissue-specific promoters and two polyA-addition sites. Further, dystrophin RNA is differentially spliced, producing a range of different transcripts, encoding a large set of protein isoforms. The terms “dystrophyn protein”, “dystrophin polypeptide”, “dystrophin RNA” and “dystrophin transcript” refer to any and all isoforms expressed by the dystrophin gene within cells of a subject (including any allelic variants).

The human Dystrophin gene measures about 2.4 Mb, and was identified through a positional cloning approach, based on the isolation of the gene responsible for Duchenne (DMD) and Becker (BMD) Muscular Dystrophies. In general, DMD patients carry mutations which cause premature translation termination (nonsense or frame shift mutations), while BMD patients carry mutations resulting in a dystrophin that is reduced either in size (from in-frame deletions) or in expression level.

Mutations in the DYS gene have also been linked to X-linked dilated cardiomyopathy and familial dilated cardiomyopathy. More than 30 mutations in the DMD gene can cause an X-linked form of familial dilated cardiomyopathy. This heart condition enlarges and weakens the cardiac muscle, preventing the heart from pumping blood efficiently. Although dilated cardiomyopathy is a sign of Duchenne and Becker muscular dystrophy (described above), X-linked dilated cardiomyopathy is typically not associated with weakness and wasting of skeletal muscles. The mutations that cause X-linked dilated cardiomyopathy preferentially affect the activity of dystrophin in cardiac muscle cells. As a result of these mutations, affected individuals typically have little or no functional dystrophin in the heart. Without enough of this protein, cardiac muscle cells become damaged as the heart muscle repeatedly contracts and relaxes. The damaged muscle cells weaken and die over time, leading to the heart problems characteristic of X-linked dilated cardiomyopathy. Generally, in X-linked dilated cardiomyopathy enough of this protein is present to prevent weakness and wasting of the skeletal muscles.

Familial dilated cardiomyopathy is characterized by a heart muscle which becomes thin and weakened in at least one chamber of the heart, causing the open area of the chamber to become enlarged (dilated). As a result, the heart is unable to pump blood as efficiently as usual. To compensate, the heart attempts to increase the amount of blood being pumped through the heart, leading to further thinning and weakening of the cardiac muscle. Over time, this condition results in heart failure. It usually takes many years for symptoms of familial dilated cardiomyopathy to cause health problems. They typically begin in mid-adulthood, but can occur at any time from infancy to late adulthood. Signs and symptoms of familial dilated cardiomyopathy can include an irregular heartbeat (arrhythmia), shortness of breath (dyspnea), extreme tiredness (fatigue), fainting episodes (syncope), and swelling of the legs and feet. In some cases, the first sign of the disorder is sudden cardiac death. The severity of the condition varies among affected individuals, even in members of the same family.

As indicated above, in a particular embodiment, the present invention uses the CRISPR system to introduce further mutations within exons or introns of a mutated dystrophin gene within a cell. The CRISPR system is a defense mechanism identified in bacterial species [37-42]. It has been modified to allow gene editing in mammalian cells. The modified system still uses a Cas9 nuclease to generate double-strand breaks (DSB) at a specific DNA target sequence [43, 44]. The recognition of the cleavage site is determined by base pairing of the gRNA with the target DNA and the presence of a trinucleotide called PAM (protospacer adjacent motif) juxtaposed to the targeted DNA sequence [45]. This PAM is NGG for the Cas9 of S. pyogenes, the most commonly used enzyme [46, 47].

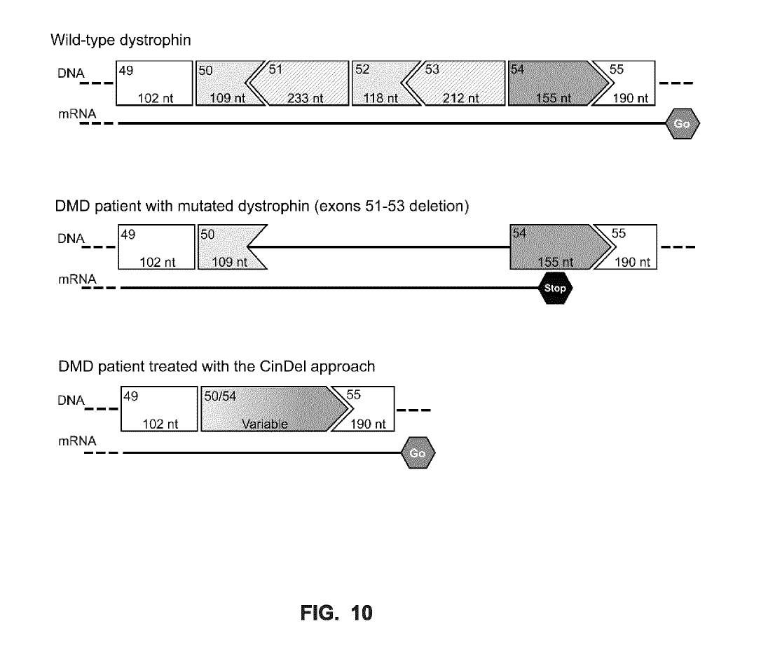

In a particular embodiment, Applicants have used various combinations of two gRNAs targeting exons 46, and 51, 46 and 53, 49 and 52, 49 and 53, 47 and 58 and 50 and 54 and introns 22 and 23, of the DYS gene both in vitro and in vivo. The in vitro experiments were done in 293T cells or in myoblasts of DMD patients having an endogenous frameshift or nonsense mutation (e.g., a deletion of exons 49-50, 51-53 or 51-56 generating a stop codon or frameshift). The in vivo experiments were done in the hDMD/mdx mouse that contains a full length human DYS gene or in the RAG/MDX mouse model comprising a mutation in exon 23. Results show that in vitro and in vivo, the gRNA combinations allowed precise DSB at 3 nucleotides upstream of the PAM and induced a large deletion. The junction between the remaining DNA sequences was achieved exactly as predicted. Using specifically selected pairs of gRNAs, it was possible to restore the reading frame resulting in the synthesis of an internally deleted DYS protein by the myotubes formed by the corrected myoblasts of DMD patients with an out-of-frame deletion. Such a CRISPR induced Deletion (CinDel) therapeutic approach can be used to restore directly in vivo the reading frame for most deletions observed in DMD patients. This approach is summarized in FIG. 10 for hybrid exon 50-54. Importantly, the deletion in the dystophin gene generated by the method of the present invention restores the correct reading frame for the protein in the endogenously mutated DYS gene and generates a shorter but functional dystrophin protein. The deletion allows maintaining the configuration of a normal spectrin-like repeat where hydrophobic amino acids are localized in position “a” and “d” of the heptad motif.

As indicated above, polypeptides (e.g., CRISPR nucleases) and nucleic acids encoding gRNAs and nucleases or nickases (e.g., Cas9 or Cpf1) of the present invention may be delivered into cells using various methods. These methods may employ one or more various viral vectors or delivery agents such as the peptide based shuttles (Feldan shulltles-discussed above).

Accordingly, preferably, the above-mentioned vector is a viral vector for introducing the gRNA and/or nuclease of the present invention in a target cell. Non-limiting examples of viral vectors include retrovirus, lentivirus, Herpes virus, adenovirus or Adeno Associated Virus, as well known in the art.

Modified AAV vector which preferably targets one or more cell types affected in DMD subjects are preferably used in accordance with the present invention. In an embodiment, the cell type is a muscle cell, in a further embodiment, a myoblast. Accordingly, the modified AAV vector may have enhanced cardiac, skeletal muscle, neuronal, liver, and/or pancreatic tissue (Langerhans cells) tropism. The modified AAV vector may be capable of delivering and expressing the at least one gRNA and nuclease of the present invention in the cell of a mammal. For example, the modified AAV vector may be an AAV-SASTG vector (Piacentino et al. (2012) Human Gene Therapy 23:635-646). The modified AAV vector may deliver gRNAs and nucleases to neurons, skeletal and cardiac muscle, and/or pancreas (Langerhans cells) in vivo. The modified AAV vector may be based on one or more of several capsid types, including AAVI, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5 and AAV/SASTG vectors that efficiently transduce skeletal muscle or cardiac muscle by systemic and local delivery. In an embodiment, the modified AAV vector is a AAV-DJ. In an embodiment, the modified AAV vector is a AAV-DJ8 vector. In an embodiment, the modified AAV vector is a AAV2-DJ8 vector.

In yet another aspect, the present invention provides a cell (e.g., a host cell) comprising the above-mentioned nucleic acid and/or vector. The invention further provides a recombinant expression system, vectors and host cells, such as those described above, for the expression/production of a recombinant protein, using for example culture media, production, isolation and purification methods well known in the art.

In another aspect, the present invention provides a composition (e.g., a pharmaceutical composition) comprising the above-mentioned gRNA and/or CRISPR nuclease (e.g., Cas9 or Cpf1), or nucleic acid(s) encoding same or vector(s) comprising such nucleic acid(s). In an embodiment, the composition further comprises one or more pharmaceutically acceptable carriers, excipients, and/or diluents.

As used herein, “pharmaceutically acceptable” (or “biologically acceptable”) refers to materials characterized by the absence of (or limited) toxic or adverse biological effects in vivo. It refers to those compounds, compositions, and/or dosage forms which are, within the scope of sound medical judgment, suitable for use in contact with the biological fluids and/or tissues and/or organs of a subject (e.g., human, animal) without excessive toxicity, irritation, allergic response, or other problem or complication, commensurate with a reasonable benefit/risk ratio.

The present invention further provides a kit or package comprising at least one container means having disposed therein at least one of the above-mentioned gRNAs, nucleases, vectors, cells, targeting systems, combinations or compositions, together with instructions for restoring the correct reading frame of a DYS gene in a cell or for treatment of DMD in a subject.

The present invention is illustrated in further details by the following non-limiting examples.

### Example 1

### Materials and Methods for Examples 1 to 7

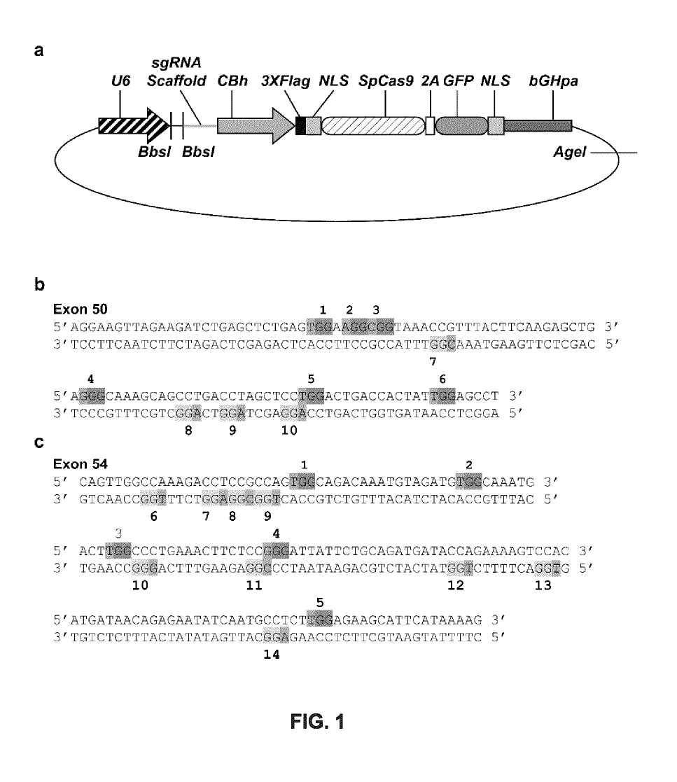

Identification of targets and gRNA cloning. The plasmid pSpCas(BB)-2A-GFP (pX458) (Addgene plasmid #48138) (FIG. 1a) [58] containing two Bbsl restriction sites necessary for insertion of a protospacer (see below) under the control of the U6 promoter was used in the experiments shown in FIGS. 1 to 8. The pSpCas(BB)-2A-GFP plasmid also contains the Cas9, of S. pyogenes, and eGFP genes under the control of the CBh promoter; both genes are separated by a sequence encoding the peptide T2A.

The nucleotide sequences targeted by the gRNAs in exons 50 and 54 were identified using the Leiden Muscular Dystrophy website by screening for Protospacer Adjacent Motifs (PAM) in the sense and antisense strands of each exon sequence (FIG. 1b). The PAM sequence for S. pyogenes Cas9 is NGG. An oligonucleotide coding for the target sequence, and its complementary sequence, were synthesized by Integrated DNA Technologies (IDT, Coralville, Iowa) and cloned into Bbsl sites as protospacers leading to the individual production of 10 gRNAs targeting exon 50 and 14 gRNAs targeting exon 54, according to Addgene's instructions. Briefly, the oligonucleotides were phosphorylated using T4 PNK (NEB, Ipwisch, Mass.) then annealed and cloned into the Bbsl sites of the plasmid pSpCas(BB)-2A-GFP using the Quickligase (NEB, Ipwisch, Mass.). Following clone isolation and DNA amplification, samples were sequenced using the primer U6F (5′-GTCGGAACAGGAGAGCGCACGAGGGAG) (SEQ ID NO: 173) and sequencing results were analyzed using the NCBI BLAST platform (http://blast.ncbi.nlm.nih.gov/Blast.cgi).

Cell Culture. Transfection of the expression plasmid in 293T cells and in DMD patient myoblasts.

The gRNA activities were tested individually or in pairs by transfection of the pSpCas(BB)-2A-GFP-gRNA plasmid encoding each gRNA in 293T cells and in DMD myoblasts having a deletion of exons 51 to 53. The 293T cells were grown in Dulbecco's modified Eagle medium (DMEM) medium (Invitrogen, Grand Island, N.Y.) containing 10% fetal bovine serum (FBS) and antibiotics (penicillin 100 U/ml/streptomycin 100 μg/ml). DMD patient myoblasts were grown in MB-1 medium (Hyclone, Thermo Scientific, Logan, Utah) containing 15% FBS, without antibiotics. Cells in either 24-well or 6-well plates were transfected at 70-80% confluency using respectively 1 or 5 μg of plasmid DNA and 2 or 10 μl of Lipofectamine™ 2000 (Invitrogen, Carlsbad, Calif.) previously diluted in Opti-Mem™ (Invitrogen, Grand Island, N.Y.). For gRNA pair transfection, half of the DNA mixture was coming from the plasmid encoding the gRNA-50 and half from the gRNA-54. The cells were incubated at 37° C. in the presence of 5% CO₂ for 48 hours. The transfection success was evaluated by the GFP expression in the transfected cells under microscopy with a Nikon TS 100 (Eclipse, Japan).

Myoblast transfection with Lipofectamine™ 2000 following the previous standard protocol was not sufficiently effective and was improved as follows. The MB-1 medium was aspirated before transfection and myoblasts were washed once with 500 μl of 1× Hanks Balanced Salt Solution (HBSS) (Invitrogen, Grand Island, N.Y.). The complex Lipofectamine 2000 plasmid DNA (diluted in Opti-Mem™ as above) was then poured directly on cells, instead of being in media, and the cells/DNA complex was incubated at 37° C. during 15 min. After this time, the antibiotic-free medium was added to the cells and the plate was returned to the incubator for 18-24 hours. After that time, the medium was aspired and replaced with the fresh medium. The plate was incubated for another 24 hours.

Myoblast differentiation in myotubes and dystrophin expression. The DMD myoblasts (transfected with gRNA2-50 and gRNA2-54) were allowed to fuse in myotubes to induce the expression of dystrophin. To permit this myoblast fusion, the MB-1 medium (Hyclone, Thermo Scientific, Logan, Utah) was aspirated from the myoblast culture and replaced by the minimal DMEM medium containing 2% FBS (Invitrogen, Grand Island, N.Y.). Myoblasts were incubated at 37° C. in 5% CO₂ for 7 days. Untransfected myoblasts (negative control) of the DMD patient and immortalized wild-type myoblasts from a healthy donor (positive control) were also grown under the same conditions to induce their differentiation in myotubes.

Genomic DNA extraction and analysis. Forty-eight (48) hours after transfection with the pSpCas(BB)-2A-GFP-gRNA plasmid(s), the genomic DNA was extracted from the 293T or myoblasts using a standard phenol-chloroform method. Briefly, the cell pellet was resuspended in 100 μl of lysis buffer containing 10% sarcosyl and 0.5 M pH 8 ethylenediaminetetraacetic acid (EDTA). Twenty (20) μl of proteinase K (10 mg/ml) were added. The suspension was mixed by up down and incubated 10 min at 55° C. It was then centrifuged at 13200 rpm for 2 min.

The supernatant was collected in a new microfuge tube. One volume of phenol-chloroform was added and following centrifugation, the aqueous phase was recovered in a new microfuge tube and ethanol-precipitated with 1:10 volume:volume of NaCl 5 M and two volumes of 100% ethanol. The pellet was washed with 70% ethanol, centrifuged and the DNA was resuspended in 50 μl of double-distilled water. The genomic DNA concentration was assayed with a NanoDrop™ (Thermo Scientific, Logan, Utah).

To confirm the successful individual cuts or deletions, exons 50 and 54 and the hybrid exon 50-54 were then amplified by PCR. For exon 50, the sense primer targeted the end of intron 49 (called Sense 49 5′-TTCACCAAATGGATTAAGATGTTC) (SEQ ID NO: 174) and the antisense primer targeted the start of intron 50 (called Antisense 50 5′-ACTCCCCATATCCCGTTGTC) (SEQ ID NO: 175). For exon 54, the forward and reverse primers targeted respectively the end of the intron 53 (called Sense 53 5′-GTTTCAAGTGATGAGATAGCAAGT) (SEQ ID NO: 176) and the start of intron 54 (called Antisense 54 5′-TATCAGATAACAGGTAAGGCAGTG) (SEQ ID NO: 177). For the hybrid exon 50-54, the forward Sense 49 and reverse Antisense 54 were used. All PCR amplifications were performed in a thermal cycler C1000 Touch of BIO RAD (Hercules, Calif.) with the Phusion high fidelity polymerase (Thermo scientific, EU, Lithuania) using the following program for exon 50, exon 54 and the hybrid exon 50-54: 98° C./10 sec, 58° C./20 sec, 72° C./1 min for 35 cycles.

The amplicons of individual exons 50 and 54 were used to perform the Surveyor assay. The first part of the test was the hybridization of amplicons using the slow-hybridization program (denaturation at 95° C. followed by gradual cooling of the amplicons) with BIO RAD thermal cycler C1000Touch (Hercules, Calif.). Subsequently, the amplicons were digested with nuclease Cel (Integrated DNA Technologies, Coralville, Iowa) in the thermal cycler at 42° C. for 25 min. The digestion products were visualized on agarose gel 1.5%

Cloning and sequencing of the hybrid exons. The amplicon of hybrid exons obtained by the amplification of genomic DNA extracted from 293T cells or myoblasts transfected with 2 different pSpCas(BB)-2A-GFP-gRNAs was purified by gel extraction (Thermo Scientific, EU, Lithuania). The bands of about 480 to 655 bp were cloned into the linearized cloning vector pMiniT (NEB, Ipwisch, MA). On day 3, the plasmid DNA was extracted with the Miniprep Kit (Thermo Scientific, EU, Lithuania) and the cloning vector was digested simultaneously with EcoRI and PstI to confirm the insertion of the amplicon. In the cloning vector pMiniT, the insert was flanked by two EcoRI restriction sites. Digestion with EcoRI generated two fragments of 2500 bp (plasmid without insert) and of 480 to 655 bp (amplicon inserted). It should be noted that there was a PstI restriction site in the remaining part of exon 54. A PstI digestion generated two fragments. The clones, which gave after double digestion with EcoRI and PstI these two fragments, were sent for sequencing using primers provided by the manufacturer (NEB, Ipwisch, MA). Sequencing results were analyzed with the NCBI BLAST platform (http://blast.ncbi.nlm.nih.gov/Blast.cgi) and the Expert Protein Analysis System (ExPASy) platform (http://www.expasy.org). This software allowed the visualization both the nucleotide sequences of the hybrid exon 50-54 and of the corresponding amino acid sequences.

In vivo mouse assay. Sperm from transgenic hDMD mice expressing the full-length human dystrophin gene were inseminated [59]. The hDMD mice were crossed with mdx mice to produce the hDMD/mdx mice.

Forty (40) μg of pSpCas-2A-GFP-gRNAs (20 μg gRNA2-50 and 20 μg gRNA2-54) were suspended in 20 μl of double-distilled water and mixed with 20 μl of Tyrode's buffer (119 mM NaCl, 5 mM KCl, 25 mM HEPES buffer, 2 mM CaCl₂ 2 mM MgCl₂, 6 g/L glucose, pH was adjusted to 7.4 with NaOH, Sigma-Aldrich). The hDMD/mdx mice were electrotransferred with an Electro Square Porator (Model ECM630, BTX Harvard Apparatus, St-Laurent, Canada) following a single transcutaneous longitudinal injection in the Tibialis anterior (TA) of the pSpCas(BB)-2A-GFP plasmids. An electrode electrolyte cream (Teca, Pleasantville, N.Y.) was applied on the skin to favor the passage of the electric field between the two electrode plates. Muscles were submitted to electric field (8 pulses of 20 ms duration spaced by 1 s). The voltage was adjusted at 100 volts/cm depending the width of the mice leg. Electroporated and control mice were sacrificed 7 days later. Genomic DNA was extracted with phenol-chloroform method as above and DNA analysis performed as previously described.

Protein analysis. Myotubes were harvested and proteins were extracted with the methanol-chloroform method. Briefly, cell pellets were resuspended in lysis buffer containing 75 mM Tris-HCl pH 7.4, 1 mM dithiotreitol (DTT), 1 mM phenylmethylsulfonyl fluoride (PMSF) and 1% sodium dodecyl sulfate (SDS). Protein extracts were dried with the speed vacuum Univapo 100 ECH (Uniequip, Martinsried, Germany) to remove all traces of methanol. Samples were then diluted in a buffer containing 0.5% mercaptoethanol and heated at 95° C. for 5 min. The protein concentrations were assayed by Amido Black using Imager2200 AlphaDigiDoc (Alpha Innotech, Fisher Scientific, Suwanee, Ga.).

Seventy-five (75) μg of protein of each sample were separated on a 7% polyacrylamide gel and transferred onto nitrocellulose membrane at 4° C. for 16 hrs. In order to detect dystrophin on the membrane, a primary mouse monoclonal antibody (cat#NCL-DYS2, Leica Biosystems, Newcastle, UK) recognizing the C-terminus of the human dystrophin was used. The antibody was diluted 1:25 in 0.1×PBS containing 5% milk and 0.05% Tween20 and incubated at 4° C. for 16 hrs.

### Example 2

### Dystrophin Exon Targeting in DMD Myoblasts Using the SpCAS9/CRISPR System

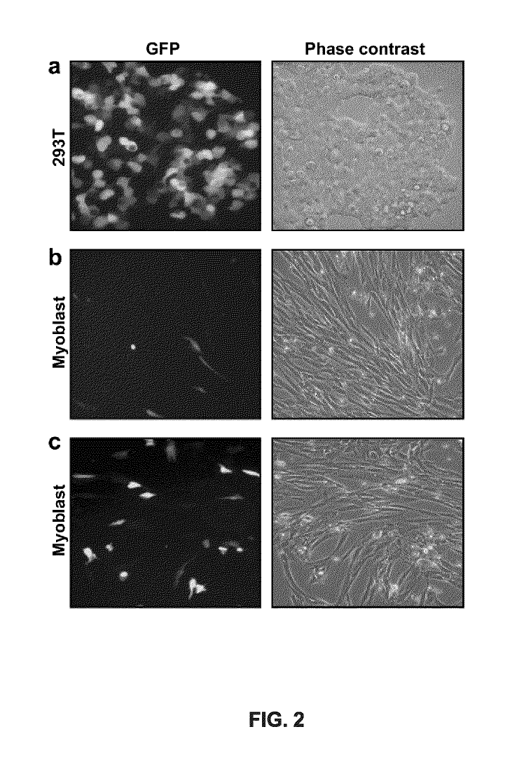

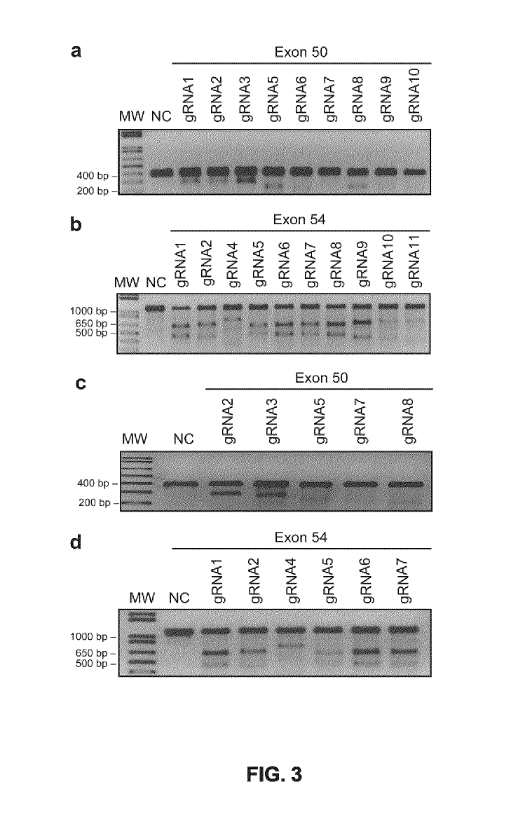

Twenty-four different pSpCas(BB)-2A-GFP-gRNA plasmids (FIG. 1a) were made: 10 containing gRNAs targeting different sequences of the exon 50 of the DYS gene and 14 containing gRNAs targeting the exon 54 (Table 3 and FIGS. 1b-c). To test the activity of these gRNAs, these plasmids were first transfected in 293T cells. Under standard transfection conditions, 80% of cells showed expression of the GFP confirming the effectiveness of the transfection (FIG. 2a). The DNA from those cells was extracted 48 hours after transfection. The exon 50 of the DYS was amplified by PCR using primers Sense 49 and Antisense 50 and exon 54 was amplified with primers Sense 53 and Antisense 54 (see Example 1 for details on primer sequences). The presence of INDELs, produced by non-homologous end-joining (NHEJ) following the DSBs generated by the gRNAs and the Cas9, was detected using the Surveyor/Cel I enzymatic assay (FIGS. 3a-b). An expected pattern of three bands was detected with most gRNAs; the upper band representing the uncut PCR product and the two lowest bands the Cel I products whose lengths are related to the guide used to induce the DSB.

TABLE 3
Exemplary gRNAs targeting exons 50 and 54 of the DYS gene

| | | Strand | | SEQ ID | | |
|---|---|---|---|---|---|---|
| | | (AS = | | NOs | Cut sites | |
| | | Anti- | | Target/ | in DYS | Cut |
| gRNA# | Exon | Sense) | Target sequence | gRNA | gene | sites in amino acid sequence |
| gRNA1-50 | 50 | Sense | TAGAAGATCTGAGCTCTGAG | 82/124 | 7224-7225 | 2408 TCT (Ser):2409 GAG (Glu) |
| gRNA2-50 | 50 | Sense | AGATCTGAGCTCTGAGTGGA | 83/125 | 7228-7229 | 2410 T:GG (Trp) |
| gRNA3-50 | 50 | Sense | TCTGAGCTCTGAGTGGAAGG | 84/126 | 7231-7232 | 2411 A:AA (Lys) |
| gRNA4-50 | 50 | Sense | CCGTTTACTTCAAGAGCTGA | 85/127 | 7258-7259 | 2420 C:TG (Leu) |
| gRNA5-50 | 50 | Sense | AAGCAGCCTGACCTAGCTCC | 86/128 | 7283-7284 | 2428 GC:T (Ala) |
| gRNA6-50 | 50 | Sense | GCTCCTGGACTGACCACTAT | 87/129 | 7298-7299 | 2433 AC:T (Thr) |
| gRNA7-50 | 50 | AS | CCCTCAGCTCTTGAAGTAAA | 88/130 | 7247-7248 | 2416 TT:A (Leu) |
| gRNA8-50 | 50 | AS | GTCAGTCCAGGAGCTAGGTC | 89/131 | 7278-7279 | 2426 GAC (Asp):2427 CTA (Leu) |
| gRNA9-50 | 50 | AS | TAGTGGTCAGTCCAGGAGCT | 90/132 | 7283-7284 | 2428 GC:T (Ala) |
| gRNA10-50 | 50 | AS | GCTCCAATAGTGGTCAGTCC | 91/133 | 7290-7291 | 2430 GGA (Gly):2431 CTG (Leu) |
| gRNA1-54 | 54 | Sense | TGGCCAAAGACCTCCGCCAG | 92/134 | 7893-7894 | 2631 CGC (Arg):2632 CAG (Gln) |
| gRNA2-54 | 54 | Sense | GTGGCAGACAAATGTAGATG | 93/135 | 7912-7913 | 2638 G:AT (Asp) |
| gRNA3-54 | 54 | Sense | TGTAGATGTGGCAAATGACT | 94/136 | 7924-7925 | 2642 G:AC Asp) |
| gRNA4-54 | 54 | Sense | CTTGGCCCTGAAACTTCTCC | 95/137 | 7941-7942 | 2648 C:TC (leu) |
| gRNA5-54 | 54 | Sense | CAGAGAATATCAATGCCTCT | 96/138 | 8004-8005 | 2668 GCC (Ala):2669 TCT (Ser) |
| gRNA6-54 | 54 | AS | CTGCCACTGGCGGAGGTCTT | 97/139 | 7885-7886 | 2629 G:AC (Asp) |
| gRNA7-54 | 54 | AS | CATTTGICTGCCACTGGCGG | 98/140 | 7892-7893 | 2631 CG:C (Arg) |
| gRNA8-54 | 54 | AS | CTACATTTGTCTGCCACTGG | 99/141 | 7895-7896 | 2632 CA:G (Gln) |
| gRNA9-54 | 54 | AS | CATCTACATTTGTCTGCCAC | 100/142 | 7898-7899 | 2633 TG:G (Trp) |
| gRNA10-54 | 54 | AS | ATAATCCCGGAGAAGTTTCA | 101/143 | 7936-7937 | 2646 A:AA (Lys) |
| gRNA11-54 | 54 | AS | TATCATCTGCAGAATAATCC | 102/144 | 7949-7950 | 2650 GA:T (Asp) |
| gRNA12-54 | 54 | AS | TGTTATCATGTGGACTTTTC | 103/145 | 7972-7973 | 2658 A:AA (Lys) |
| gRNA13-54 | 54 | AS | TGATATATCATTICTCTGIG | 104/146 | 7982-7983 | 2661 AT:G (Met) |
| gRNA14-54 | 54 | AS | TTTATGAATGCTTCTCCAAG | 105/147 | 8008-8009 | 2670 T:GG (Trp) |

The gRNAs were also subsequently tested individually in immortalized myoblasts from a DMD patient having a deletion of exons 51 through 53. Unfortunately, transfection efficiency was very low in myoblasts under the standard Lipofectamine™ 2000 transfection [14] (FIG. 2b). However, the protocol was improved and we were able to see approximately 20 to 25% of myoblasts expressing GFP (FIG. 2c). The Surveyor assay revealed the presence of INDELs in amplicons of exons 50 (FIG. 3c) and 54 (FIG. 3d) obtained from these myoblasts.

### Example 3

### Testing of gRNA Pairs in the SpCAS9/CRISPR System

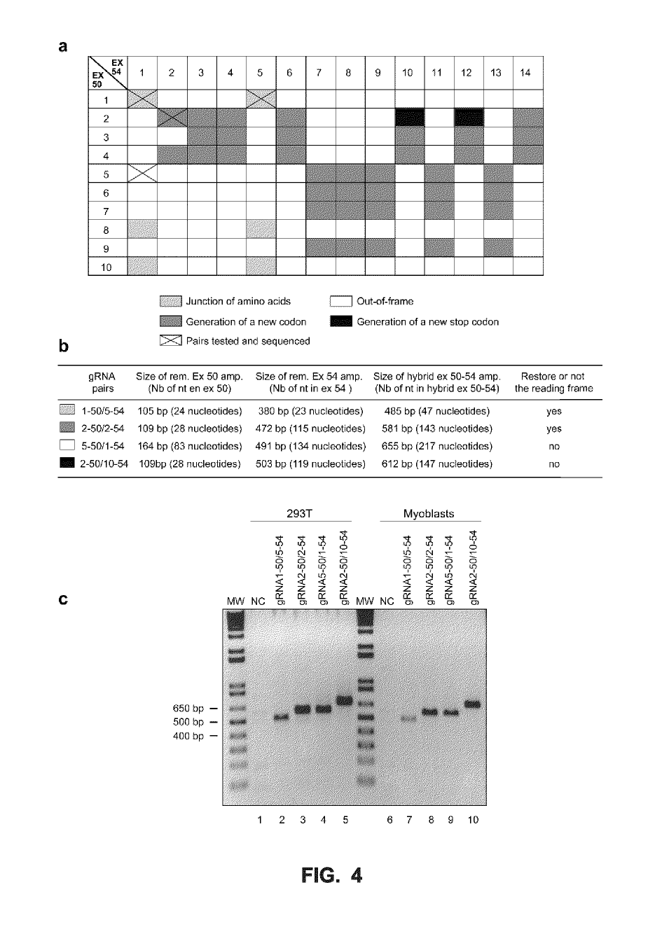

Given that the CRISPR/Cas9 induces a DSB at exactly 3 bp from the PAM in the 5′ direction, it was possible to predict the consequence of cutting of the exons 50 and 54 with the various pairs of gRNAs. This analysis predicted four possibilities, as illustrated in FIG. 4a and detailed in Table 4: 1) the total number of coding nucleotides, which are deleted (i.e., the sum of the nucleotides of exons 51, 52 and 53 and the portions of exons 50 and 54, which are deleted) is a multiple of three and the junction of the remains of 50 exons and 54 does not generate a new codon, 2) the number of deleted nucleotides coding for DYS is a multiple of three but a new codon, derived from the junction of the remains of 50 exons and 54, encodes a new amino acid, 3) the number of coding nucleotides, which are deleted is not a multiple of three resulting in an incorrect reading frame of the DYS gene; and 4) the sum of deleted nucleotides coding for DYS is a multiple of three, but the new codon, formed by the junction of the remaining parts of exons 50 and 54, is a stop codon.

TABLE 4
Hybrid exon junctions following first and second cuts in exons 50 and 54 with various gRNA pairs

| | End of | Beginning of | | New codon | New amino acid |
|---|---|---|---|---|---|
| Combination | Exon 50 remain | Exon 54 remain | Observation | generated | generated |
| gRNA1 Ex 50/gRNA1 Ex 54 | Ser 2408 | Gln 2632 | Junction Ser 2408-Gln 2632 | None | None |
| gRNA1 Ex 50/gRNA5 Ex 54 | Ser 2408 | Ser 2669 | Junction Ser 2408-Ser2669 | None | None |
| gRNA2 Ex 50/gRNA2 Ex 54 | T | AT | T + AT = TAT | TAT | Tyr |
| gRNA2 Ex 50/gRNA3 Ex 54 | T | AC | T + AC = TAC | TAC | Tyr |
| gRNA2 Ex 50/gRNA6 Ex 54 | T | AC | T + AC = TAC | TAC | Tyr |
| gRNA2 Ex 50/gRNA 14 Ex 54 | T | GG | T + GG = TGG | TGG | Trp |
| gRNA3 Ex 50/gRNA2 Ex 54 | A | AT | A + AA = AAT | AAT | Asn |
| gRNA3 Ex 50/gRNA3 Ex 54 | A | AC | A + AC = AAC | AAC | Asn |
| gRNA3 Ex 50/gRNA6 Ex 54 | A | AC | A + AC = AAC | AAC | Asn |
| gRNA3 Ex 50/gRNA10 Ex 54 | A | AA | A + AA = AAA | AAA | Lys |
| gRNA3 Ex 50/gRNA12 Ex 54 | A | AA | A + AA = AAA | AAA | Lys |
| gRNA3 Ex 50/gRNA14 Ex 54 | A | GG | A + GG = AGG | AGG | Arg |
| gRNA4 Ex 50/gRNA2 Ex 54 | C | AT | C + AT = CAT | CAT | His |
| gRNA4 Ex 50/gRNA3 Ex 54 | C | AC | C + AC = CAC | CAC | His |
| gRNA4 Ex 50/gRNA6 Ex 54 | C | AC | C + AC = CAC | CAC | His |
| gRNA4 Ex 50/gRNA 10 Ex 54 | C | AA | C + AA = CAA | CAA | Gln |
| gRNA4 Ex 50/gRNA12 Ex 54 | C | AT | C + AT = CAT | CAT | His |
| gRNA4 Ex 50/gRNA14 Ex 54 | C | GG | C + GG = CGG | CGG | Arg |
| gRNA5 Ex 50/gRNA7 ex 54 | GC | C | GC + C = GCC | GCC | Ala |
| gRNA5 Ex 50/gRNA 8ex 54 | GC | G | GC + G = GCG | GCG | Ala |
| gRNA5 EX 50/gRNA9 Ex 54 | GC | G | GC + G = GCG | GCG | Ala |
| gRNA5 Ex 50/gRNA11 Ex 54 | GC | T | GC + T = GCT | GCT | Ala |
| gRNA5 Ex50/gRNA13 EX 54 | GC | G | GC + G = GCG | GCG | Ala |
| gRNA6 Ex 50/gRNA7 Ex 54 | AC | C | AC + C = ACC | ACC | Thr |
| gRNA6 Ex 50/gRNA8 Ex 54 | AC | G | AC + G = ACG | ACG | Thr |
| gRNA6 Ex 50/gRNA9 Ex 54 | AC | G | AC + G = ACG | ACG | Thr |
| gRNA6 Ex 50/gRNA11 Ex 54 | AC | T | AC + T = ACT | ACT | Thr |
| gRNA6 Ex 50/gRNA13 Ex 54 | AC | G | AC + G = ACG | ACG | Thr |
| gRnA7 Ex 50/gRNA7 Ex 54 | TT | C | TT + C = TTC | TTC | Phe |
| gRNA7 Ex 50/gRNA8 Ex 54 | TT | G | TT + G = TTG | TTG | Leu |
| gRNA7 Ex 50/gRNA9 Ex 54 | TT | G | TT + G = TTG | TTG | Leu |
| gRNA7 Ex 50/gRNA11 Ex 54 | TT | T | TT + T = TTT | TTT | Phe |
| gRNA7 Ex 50/gRNA13 Ex 54 | TT | G | TT + G = TTG | TTG | Leu |
| gRNA8 Ex 50/gRNA1 Ex 54 | Asp2426 | Gln2632 | Junction Asp2426-Gln2632 | None | None |
| gRNA8 Ex 50/gRNA5 Ex 54 | Asp2426 | Ser 2669 | Junction Asp2426-Ser2669 | None | None |
| gRNA9 Ex 50/gRNA7 Ex 54 | GC | C | GC + C = GCC | GCC | Ala |
| gRNA9 eEx 50/gRNA8 Ex 54 | GC | G | GC + G = GCG | GCG | Ala |
| gRNA9 Ex 50/gRNA9 Ex 54 | GC | G | GC + G = GCG | GCG | Ala |
| gRNA9 Ex 50/gRNA11 Ex 54 | GC | T | GC + T = GCT | GCT | Ala |
| gRNA9 Ex 50/gRNA13Ex 54 | GC | G | GC + G = GCG | GCG | Ala |
| gRNA10 Ex 50/gRNA1 Ex 54 | Gly2430 | Gln2632 | Junction Gly 2430-Gln2632 | None | None |
| gRNA10 Ex 50/gRNA5 Ex 54 | Gly2430 | Ser 2669 | Junction Gly 2430-Ser2669 | None | None |

The deletion of part of the DYS gene was investigated by transfecting 293T cells and human myoblasts with different pairs of plasmids encoding gRNAs: one targeting exon 50 and the other the exon 54 (FIGS. 4b and 4c). To detect successful deletions, genomic DNA was extracted from these transfected and non-transfected cells 48 hours later and amplified by PCR using primers Sense 49 and Antisense 54 (see Example 1 for details regarding primer sequences). No amplification was obtained from DNA extracted from untransfected cells (FIG. 4c, lanes 1 and 6) because of the expected amplicon size (about 160 Kbp) of the wild-type DYS gene (i.e., exon 50 to exon 54) is too big. However, amplicons, named hybrid exons, of the expected sizes were obtained when a pair of gRNAs was used (FIG. 4b, lanes 2-5 and lanes 7-10), confirming the excision of the 160 Kbp sequence in 293T cells.

As shown in FIG. 4b, several different gRNA pairs (targeting exons 50 and 54) were tested and all produced exactly the expected modification of the DYS gene according to the four possibilities explained above.

### Example 4

### Characterization of Hybrid Exon 50-54 in 293T Cells (SpCAS9/CRISPR System)

The amplicons obtained following transfection of the gRNA pairs were gel purified and cloned into the pMiniT plasmid, transformed in bacteria and clones were screened for successful insertions. Positive clones, according to the digestion pattern, were sent for sequencing to demonstrate the presence of a hybrid exon formed by the fusion of a part of exon 50 with a portion of exon 54. For example, in 100% (7/7) of sequences obtained for the gRNA5-50 and gRNA1-54 pair, the DYS gene was cut in both exons at exactly 3 nucleotides in the 5′ direction from the PAM (data not shown). This exercise was repeated with different pairs of gRNAs and for each functional gRNA pair, the CinDel technique removed successfully a portion of about 160 100 bp in the DYS gene of 293T cells.

### Example 5

### Characterization of Hybrid Exon 50-54 in Myoblasts (SpCAS9/CRISPR System)

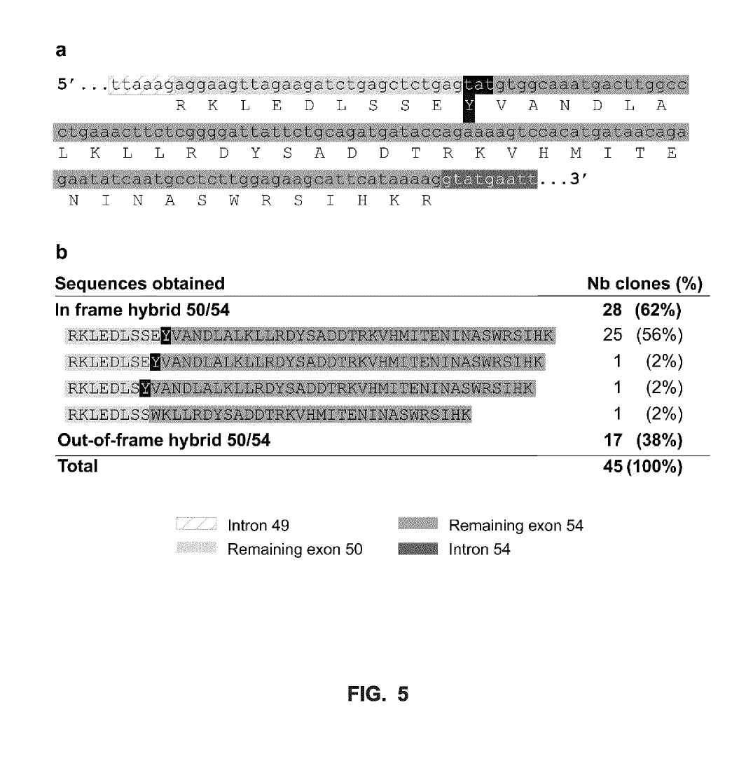

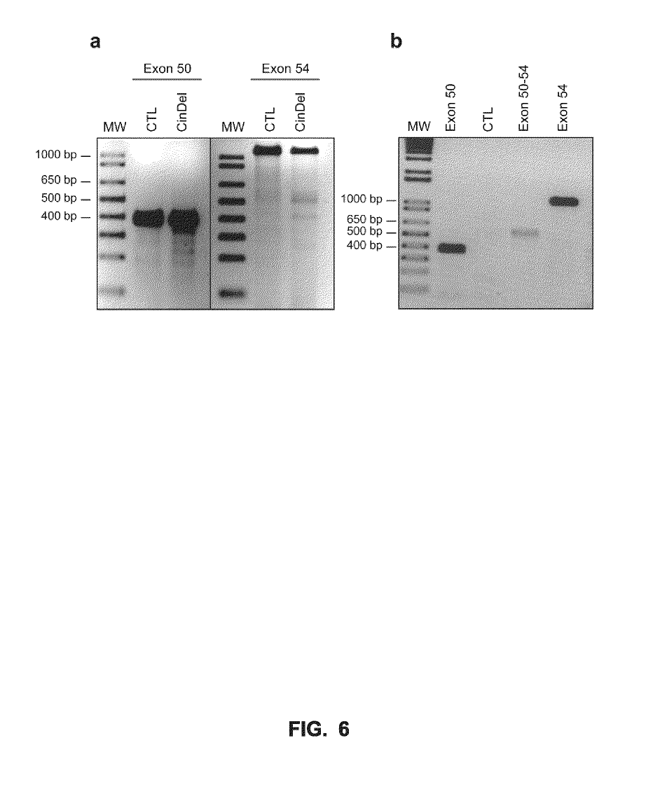

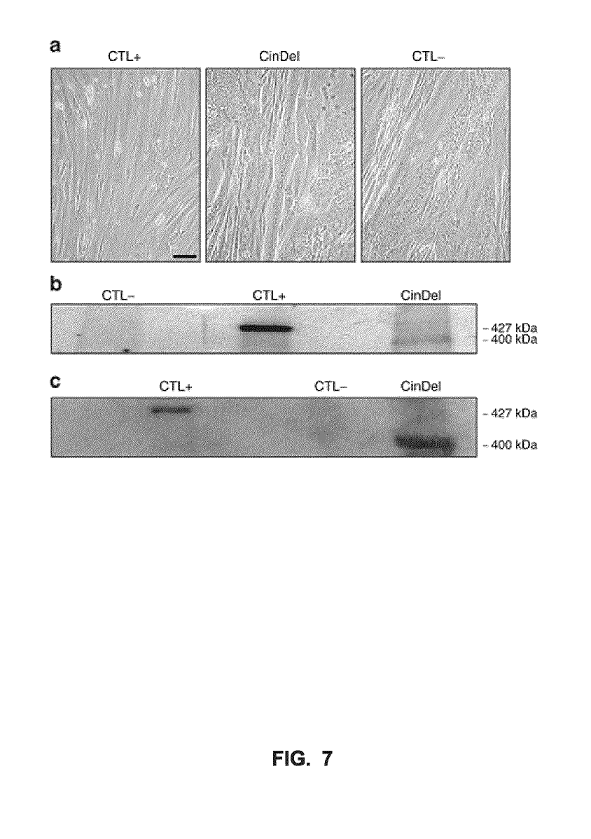

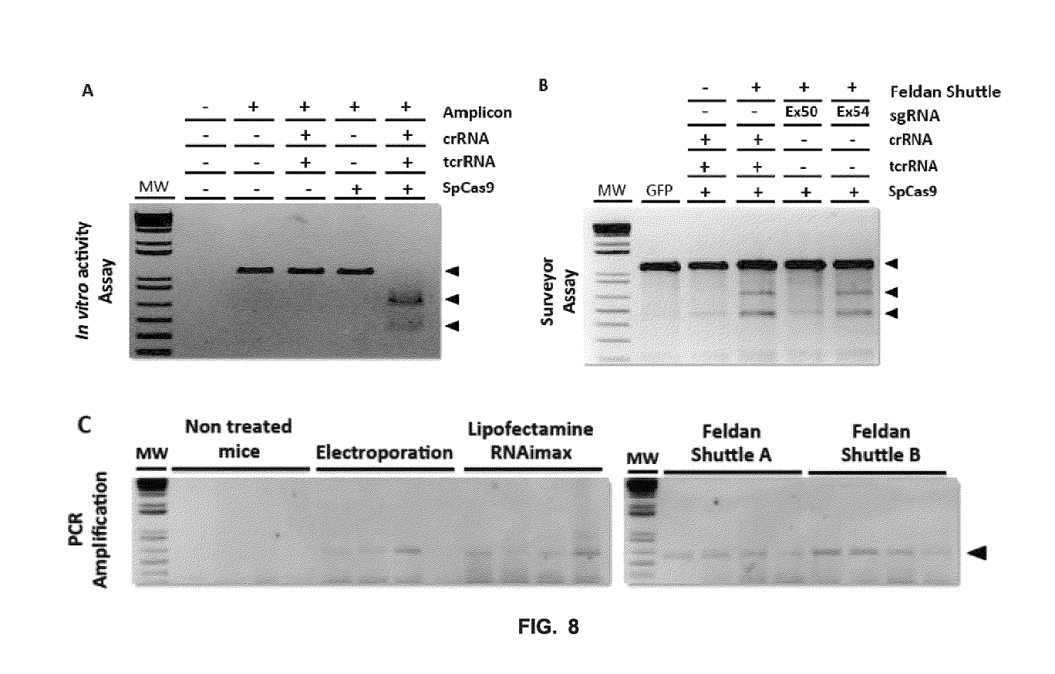

We also wanted to confirm the accuracy of cuts produced by the Cas9 from our expression plasmids in the myoblasts of a DMD patient already having a deletion of exons 51 to 53. We thus transfected the gRNA 2-50 and gRNA 2-54 pair previously characterized to produce a deletion in the DYS gene restoring the reading frame. As control, we also used another gRNA pair (i.e., gRNA5-50 and gRNA1-54) that should not restore the reading frame. As in 293T, genomic DNA of these myoblasts was extracted 48 hours later and amplified with primers Sense 49 and Antisense 54 and amplicons were cloned into the plasmid pMiniT. The plasmids were extracted from bacterial clones, screened according to their digestion pattern and positive clones were sequenced. The sequences of 45 clones were analyzed for the gRNA2-50 and gRNA2-54 pair and the most abundant product (25/45, i.e. 56%) contained exactly the expected junction between the remaining parts exons 50 and 54 to produce a 141 bp hybrid exon (FIGS. 5a and 5b). For 60% (27/45), a new codon (Y) was created (FIGS. 5a and 5b). A percentage of 62% (28/35) was detected as in-frame hybrid exons (FIG. 5b) and 38% (17/45) as out-of-frame hybrid exons (FIG. 5b).

For the second gRNA pair (gRNA5-50 and gRNA1-54), the plasmids were extracted from eight bacterial clones and sequenced. The sequence of these clones also demonstrated that 75% (6 out of 8) of these hybrid exons 50-54 (amplicon 655 bp) contained the expected reading frame shift. One of the two remaining clones showed a 1 bp insertion in addition of the expected deletion, this restored the DYS reading frame. Another clone showed an additional deletion of 11 bp that did not restore the reading frame.

### Example 6

### In Vivo Correction in the HDMD/MDX Mouse (SpCAS9/CRISPR System)

As the CinDel method was effective in 293T cells and in DMD myoblasts in culture, plasmids coding for a pair of gRNAs were electroporated in the Tibialis anterior (TA) of a hDMD/mdx mouse to confirm CinDel effects in vivo. Genomic DNA was extracted 7 days later from the gRNA2-50/2-54 electroporated TA and from a non-electroporated TA. Exons 50 and 54 of the human dystrophin gene were PCR amplified. We were able to detect additional bands following digestion of the amplicon of these exons by the Cell enzyme of the Surveyor assay (FIG. 6a, CinDel lanes). These results confirmed that both gRNAs were able to induce mutations of their targeted exon in vivo. Moreover, the hybrid exon 50-54 was also PCR amplified (FIG. 6b, lane 3) demonstrating that both gRNAs were able to cut simultaneously in vivo leading to a deletion of more than 160 kb. The amplicons of the hybrid exon 50-54 were cloned in bacteria and 11 clones were sequenced. The sequences of 7 of these clones were the same as those of the obtained for in vitro experiments with the same gRNA pair (FIG. 5b), thus 64% (7 out of 11) of the sequences showed a correct restoration of the reading frame in vivo.

### Example 7

### DYS Expression in Myotubes Formed by Genetically Corrected Myoblasts (SpCAS9/CRISPR System)

In order to verify whether the CinDel gene therapy method was efficient in restoring the expression of the DYS protein, DMD myoblasts transfected with gRNA2-50 and gRNA2-54 were differentiated into myotubes in vitro. The proteins from the resulting myotubes (FIG. 7a) were extracted after 7 days in the fusion medium. A western blot confirmed the presence of a truncated (Trunc.) DYS protein with a molecular weight of about 400 kDa (FIG. 7b, lane 3). The size of this protein corresponds to the weight expected in the absence of exons 51-53 and of portions of exons 50 and 54, while the molecular weight of the full-length (FL) DYS protein is 427 kDa in normal myotubes (FIG. 7b, lane 2). No DYS protein was detected in proteins extracted from the DMD myotubes that had not been genetically corrected (FIG. 7b, lane 1). This result indicates that myotubes formed in vitro by myoblasts of a DMD patient in which the reading frame has been restored by the CinDel are able to express an internally truncated DYS protein.

### Example 8

### Materials and Methods for Examples 9 to 23

Identification of targets and gRNA cloning. The plasmid pX601-AAV-CMV::NLS-SaCas9-NLS-3xHA-bGHpA;U6::Bsal-sgRNA (Addgene plasmid #61591; SEQ ID NO: 167) containing two Bsal restriction sites necessary for insertion of a protospacer (see below) under the control of the U6 promoter was used in our study. The pX601 plasmid also contains the Cas9 of S. aureus.

The nucleotide sequences targeted by the gRNAs along exons 46 and 58 were identified using the benchling software website by screening for Protospacer Adjacent Motifs (PAM) in the sense and antisense strands of each exon sequence. The PAM sequence for S. aureus Cas9 is NNGRRT. An oligonucleotide coding for the target sequence, and its complementary sequence, were synthesized by Integrated DNA Technologies (IDT, Coralville, Iowa) and cloned into Bsal sites as protospacers leading to the individual production of 2 gRNAs targeting exon 46, 3 gRNAs targeting exon 47, 1 gRNA targeting exon 49, 2 gRNAs targeting exon 51, 2 gRNAs targeting exon 52, 5 gRNAs targeting exon 53 and 3 gRNAs targeting exon 58 (see Table 6 below for sequences), according to Addgene's instructions. Briefly, the oligonucleotides were phosphorylated using T4 PNK (NEB, Ipwisch, MA) then annealed and cloned into the Bsal sites of the plasmid pX601-AAV-CMV::NLS-SaCas9-NLS-3xHA-bGHpA;U6::Bsal-sgRNA using the Quickligase (NEB, Ipwisch, MA). Following clone isolation and DNA amplification, samples were sequenced using the primer U6F2 (5′ GAGGGCCTATTTCCCATGATT 3′) (SEQ ID NO: 178) and sequencing results were analyzed using the CLC Sequence Viewer software (CLC Bio).

Cell Culture. Transfection of the Expression Plasmid in 293T Cells and in DMD Patient Myoblasts.

The gRNA activities were tested individually or in pairs by transfection of the pX601-AAV-CMV::NLS-SaCas9-NLS-3xHA-bGHpA;U6::Bsal-sgRNA plasmid encoding each gRNA in 293T cells and in DMD myoblasts having a deletion of exons 49 to 50 or a deletion of exons 51 to 53, or a deletion of exons 51 to 56. The 293T cells were grown in Dulbecco's modified Eagle medium (DMEM) medium (Invitrogen, Grand Island, N.Y.) containing 10% fetal bovine serum (FBS) and antibiotics (penicillin 100 U/ml/streptomycin 100 μg/ml). DMD patient myoblasts were grown in MB-1 medium (Hyclone, Thermo Scientific, Logan, Utah) containing 15% FBS, without antibiotics.

293T in 24-well were transfected at 70-80% confluency using respectively 1 μg of plasmid DNA and 3 μl of Lipofectamine™ 2000 (Invitrogen, Carlsbad, Calif.) previously diluted in Opti-Mem™ (Invitrogen, Grand Island, N.Y.). For gRNA pair transfection, half of the DNA mixture was coming from the plasmid encoding a gRNA with a target sequence upstream of exon 50 and half from a gRNA with a target sequence downstream of exon50. The cells were incubated at 37° C. in the presence of 5% CO₂ for 48 hours.

Myoblast were transfected at 60-70% confluency in 6-well plates using 5 μg of plasmid DNA and 2 μL of TransfeX™ transfection reagent (ATCC® ACS-4005™) previously diluted in Opti-MEM™. The MB-1 medium was replaced by fresh medium before transfection. The complex TransfeX plasmid DNA (diluted in Opti-Mem™ as above) was then poured on cells, and the cells/DNA complex was incubated at 37° C. overnight followed by replacement of culture medium with the fresh MB-1. Cells sere incubated at 37° C. in the presence of 5% CO₂ for 48 hours.

Genomic DNA extraction and analysis. Forty-eight (48) hours after transfection with the pX601-AAV-CMV::NLS-SaCas9-NLS-3xHA-bGHpA;U6::Bsal-sgRNA plasmid(s), the genomic DNA was extracted from the 293T or myoblasts using a standard phenol-chloroform method. Briefly, the cell pellet was resuspended in 100 μl of lysis buffer containing 10% sarcosyl and 0.5 M pH 8 ethylene diamine tetra acetic acid (EDTA). Twenty (20) μl of proteinase K (10 mg/ml) were added. The suspension was mixed by up down and incubated 10-15 min at 55° C. Suspension was then centrifuged at 13200 rpm for 5 min. The supernatant was collected in a new microfuge tube. One volume of phenol-chloroform was added and following centrifugation, the aqueous phase was recovered in a new microfuge tube. Then DNA was precipitated using 1/10 volume of NaCl 5 M and two volumes of 100% ethanol followed by 5 min centrifugation ate 13200 rpm. The pellet was washed with 70% ethanol, centrifuged and the DNA was resuspended in double-distilled water. The genomic DNA concentration was assayed with a NanoDrop™ spectrophotometer (Thermo Scientific, Logan, Utah).

To confirm the successful individual cuts or deletions, exons 46, 47, 49, 51, 52, 53, 58 and the hybrid exon 46-51, 46-53, 49-52, 49-53, 47-58 were then amplified by PCR. For exon 46, the sense primer targeted the end of intron 45 (called Sense 46 5′-CCTCCCTAAGCGCTAGGGTTACAGG) (SEQ ID NO: 179) and the antisense primer targeted the start of intron 46 (called Antisense 46 5′-ACTCCCCATATCCCGTTGTC) (SEQ ID NO: 180). For exon 47, the forward and reverse primers targeted respectively the end of the intron 46 (called Sense 47 5′-GTATTTGAGGTACCACTGGGCCCTC) (SEQ ID NO: 181) and the start of intron 47 (called Antisense 47 5′-GCCACTGAGCTGGACACACGAAATG) (SEQ ID NO: 182). For exon 49, the forward and reverse primers targeted respectively the end of the intron 48 (called Sense 49 5′-GTCATGCTTCAGCCTTCTCCAGAC) (SEQ ID NO: 183) and the start of intron 49 (called Antisense 49 5′-GTTTATCCCAGGCCAGCTTTTTGC) (SEQ ID NO: 184). For exon 51, the forward and reverse primers targeted respectively the end of the intron 50 (called Sense 51 5′-GGCTTTGATTTCCCTAGGGTCCAGC) (SEQ ID NO: 185) and the start of intron 51 (called Antisense 51 5′-GGAGAAGGCAAATTGGCACAGACAA) (SEQ ID NO: 186). For exon 52, the forward and reverse primers targeted respectively the end of the intron 51 (called Sense 52 5′-GTAATCCGAGGTACTCCGGAATGTC) (SEQ ID NO: 187) and the start of intron 52 (called Antisense 52 5′-GTTTCCCCTACTCCTTCGTCTGTC) (SEQ ID NO: 188). For exon 53, the forward and reverse primers targeted respectively the end of the intron 52 (called Sense 53 5′-CACTGGGAAATCAGGCTGATGGGTG) (SEQ ID NO: 189 and the start of intron 53 (called Antisense 53 5′-GCCAAGGAAGGAGAATTGCTTGAGG) (SEQ ID NO: 190). For exon 58, the forward and reverse primers targeted respectively the end of the intron 57 (called Sense 58 5′-GGCTCACGGTATACCTCACGATCC) (SEQ ID NO: 191) and the start of intron 58 (called Antisense 58 5′-CCTCCTCACAGATAACTCCCTTTG) (SEQ ID NO: 192) For the hybrid exons 46-51, the forward Sense 46 and reverse Antisense 51 were used. For the hybrid exons 46-53, the forward Sense 46′ (5-′CACTGCGCCTGGCCAGGAATTTTTGC) (SEQ ID NO: 193) and reverse Antisense 51 were used. For the hybrid exon 47-52, the forward Sense 47 and reverse Antisense 52 were used. For the hybrid exon 49-52, the forward Sense 49 and reverse Antisense 52 were used. For the hybrid exon 49-53, the forward Sense 49 and reverse Antisense 53 were used. From 293T cells, for the hybrid exons 47-58 the primer forward Sense 47(SEQ ID NO: 181) and the primer reverse Antisense 58 (SEQ ID NO: 192) were used. From myoblasts cells, for the hybrid exons 47-58 the forward Sense 47′ (5′-CAATAGAAGCAAAGACAAGGTAGTTG) (SEQ ID NO: 194) and the reverse Antisense 58′ (5′-GCACAAACTGATTTATGCATGGTAG) (SEQ ID NO: 195) were used. From genomic DNA of mice injected with AAVs, for an optimal detection of the formation of the hybrid exons 47-58, we performed a nested-PCR. The first PCR was done using the primer forward Sense 47 (SEQ ID NO: 181) and the primer reverse Antisense 58 (SEQ ID NO: 192). The second PCR was done using the primer forward Sense 47′ (SEQ ID NO: 194) and the primer reverse Antisense 58′ (SEQ ID NO: 195). All PCR amplifications were performed in a thermal cycler C1000 Touch of BIO RAD (Hercules, Calif.) with the Phusion™ high fidelity polymerase (Thermo scientific, EU, Lithuania). Exon 46 was amplified using the following program: 98° C./10 sec, 64.5° C./30 sec, 72° C./40 sec for 35 cycles. Exons 47, 49, 51 and 53 were amplified using the following program: 98° C./10 sec, 61.2° C./30 sec, 72° C./45 sec for 35 cycles. Exons 52 and 58 were amplified using the following program: 98° C./10 sec, 63° C./30 sec, 72° C./40 sec for 35 cycles. The hybrid exons 46-51 were amplified using the following program: 98° C./10 sec, 66° C./30 sec, 72° C./30 sec for 35 cycles. The hybrid exons 46-53 were amplified using the following program: 98° C./10 sec, 65.5° C./30 sec, 72° C./40 sec for 35 cycles. The hybrid exon 47-52 was amplified using the following program: 98° C./10 sec, 61.2° C./30 sec, 72° C./30 sec for 35 cycles. The hybrid exon 49-52 was amplified using the following program: 98° C./10 sec, 66° C./30 sec, 72° C./45 sec for 35 cycles. The hybrid exon 49-53 was amplified using the following program: 98° C./10 sec, 63° C./30 sec, 72° C./45 sec for 35 cycles. From 293T cells, the hybrid exons 47-58 were amplified using the following program: 98° C./10 sec, 61.2° C./30 sec, 72° C./30 sec for 35 cycles. From myoblasts cells, the hybrid exons 47-58 were amplified using the following program: 98° C./10 sec, 63° C./30 sec, 72° C./30 sec for 35 cycles. The amplicons of individual exons 46, 47, 49, 51, 52, 53 and 58 were used to perform the Surveyor assay. There was first a hybridization step of the amplicons using a slow-hybridization program (denaturation at 95° C. for 5 min followed by gradual cooling of the amplicons) with BIO RAD thermal cycler C1000Touch™ (Hercules, Calif.). Subsequently, the amplicons were digested with nuclease Cel (Integrated DNA Technologies, Coralville, Iowa) in the thermal cycler at 42° C. for 1 hour. The digestion products were visualized on agarose gel 2%.

Cloning and sequencing of the hybrid exons. The amplicons of hybrid exons obtained by the amplification of genomic DNA extracted from 293T cells or myoblasts transfected with 2 different pX601-AAV-CMV::NLS-SaCas9-NLS-3xHA-bGHpA;U6::Bsal-sgRNA plasmid was purified using the GeneJET™ PCR Purification Kit (Thermo Scientific, EU, Lithuania). The purified PCR products were cloned into the linearized cloning vector pMiniT™ (NEB, Ipwisch, MA). Then, plasmid DNA was extracted with the Miniprep Kit (Thermo Scientific, EU, Lithuania). The clones were sent for sequencing using primers provided by the manufacturer (NEB, Ipwisch, MA). Sequencing results were analyzed with the CLC Sequence Viewer software (CLCBio).

sgRNA in vitro Transcription. SgRNAs 1-50 and 5-54 were amplified by PCR from plasmids presented in lyombe et al. (2016) using a C1000 Touch thermocycler (Bio Rad Inc., Hercules, Calif., USA). The forward primer permitted to add in 5′ the T7 promoter sequence (primers Fw-IVT-1-50 and Fw-IVT-5-54, Table 1). Reverse primer corresponds to the end of the tracrRNA (Rv-IVT-tracr, Table 5). Amplicon sequences coding for sgRNAs were purified on column (PCR Purification Kit GeneJET, Thermo Scientific Inc., Waltham, Mass., USA) and dosed with a NanoDrop™ (Thermo Scientific Inc.). Transcription was made with the kit HiScribe Quick™ T7 High Yield RNA Synthesis Kit (NEB Inc. Ipswich, Mass., USA) using 500 ng of amplicons with an incubation of 16 h at 37° C. Thereafter, a treatment with DNase I was made followed by an extraction with phenol/chloroform and a precipitation with cold ethanol. After centrifugation, pellets were resuspended in 0.1 mM EDTA solution and the RNA concentration was estimated using a NanoDrop™ (Thermo Scientific Inc.). The sgRNAs obtained were stored at −80° C.

TABLE 5
Primers used for the amplification of in vitro transcription
templates.

| Primer name | Sequence |
|---|---|
| Fw-IVT-1-50 | 5'-TAATACGACTCACTATAAGAAGATCTGAGCTCTGAGGTTTT-3' |
| (SEQ ID NO: 201) | |
| Fw-IVT-5-54 | 5'-TAATACGACTCACTATAAGAGAATATCAATGCCTCTGTTTTAG-3' |
| (SEQ ID NO: 202) | |
| Rv-IVT-tracr | 5'-AAAAAAGCACCGACTCGGTGCCA-3' |
| (SEQ ID NO: 203) | |

In vitro Activity Analysis of crRNA:tracrRNA:SpCas9 Ribonucleic Complex. In order to analyze the activity of the crRNA:tracrRNA:SpCas9 complex, exon 54 of the dystrophin gene was amplified by PCR using a C1000 Touch thermocycler. CrRNA and tracrRNA were obtained commercially from Dharmacon Inc. and Cas9 protein was obtained from Feldan Therapeutics Inc. The crRNA:tracrRNA: SpCas9 was then mixed with the amplicon of exon 54 and incubated at 37° C. for 30 min. The reaction was stopped with RNase (1 mg/mL) and heating at 56° C. for 5 min. Cleavage analysis was done by migration on a 1.5% agarose gel in 1×TBE buffer.

In vitro Protein Delivery. For experiments, HeLa cells were plated in 96 well plates at a confluence of 10000 cells per well and incubated overnight in DMEM medium (Dulbecco's Modified Eagle Medium, Invitrogen Inc. Carlsbad, Calif., USA) supplemented with 10% fetal bovine serum and 5% penicillin and streptomycin. The cells were incubated at 37° C. under an atmosphere of 5% CO2. The analysis of the specificity and efficiency of sgRNA was then done with these cells using Feldan Shuttle technology (Feldan Therapeutics Inc.). Ribonucleic complexes were made using 2.5 μM of SpCas9 protein and 2 μM of each sgRNA separately and incubated for 5 min at room temperature (RT). Complexes were incubated with Feldan Shuttle A at a concentration of 50 μM and volume was completed using PBS 1×. Mixture was added directly to the cells for exactly 1 min incubation at RT. Cells were then washed with medium once followed by a washing step with PBS 1×. Fresh medium was added and cells were incubated for 48 h before genomic DNA extraction.

In Vivo Protein Delivery

Electroporation. The Tibialis anterior (TA) muscles of hDMD mice were injected longitudinally with 40 μL of a solution containing the protein SpCas9 complexed with 2 sgRNAs and a protein delivery agent. Complexes formed with 2.5 μM of SpCas9 protein and 2 μM of sgRNA (total amount of both sgRNA used) were incubated for 5 min and electroporated (electroporator Electro Square Porator, Model ECM630, BTX Harvard Apparatus Inc. Holliston, Mass., USA). An electrolyte cream (Teca Inc. Louisville, Quebec, Canada) was applied to the mouse leg to promote the passage of electrical pulses. Eight pulses of 20 ms separated by 1 s were applied to the mouse muscle. The voltage was adjusted at 100 V/cm depending on the width of the treated mouse leg. The mice were sacrificed 72 hours later. The mice were then anesthetised with isoflurane and euthanized by CO2 inhalation. The muscles were collected and separated into 4 pieces lengthwise.

Lipofectamine™ RNAimax. 1 μM of SpCas9 protein and 0.7 μM of sgRNA (total amount of both sgRNA used) was delivered using 2 μL of Lipofectamine RNAimax (Thermo Fisher Scientific Inc.). The ribonucleic complex was initially incubated for 5 min and then mixed with Lipofectamine RNAimax, followed by another incubation of 20 min before the longitudinally injection in TA muscles of hDMD mice with a final volume of 40 μL. The mice were sacrificed 72 hours later. The muscles were collected with the same method as for the electroporation.

Feldan Shuttles. 2.5 μM of SpCas9 protein were complexed with 2 μM of sgRNAs (total amount of both sgRNA used) and then incubated for 5 min at RT. Resulting complexes were mixed with 2 different Feldan Shuttles, Feldan Shuttle A (FSA, SEQ ID NO: 196) at 50 μM or Feldan Shuttle B (FSB, SEQ ID NO: 197) at 35 μM. In both cases, the final volume of the reaction was maintained at 40 μL. This solution was injected longitudinally in the TA muscles. The mice were sacrificed 72 hours later. The muscles were collected with the same method as for the electroporation.

Genomic DNA Extraction from Muscles. Treated TA muscles were collected and separated into 4 pieces lengthwise. The fractions were subsequently treated with a lysing buffer containing proteinase K (2 mg/mL). This was followed by incubation for 16 hours at 55° C. Lysis was followed by extraction with phenol/chloroform to obtain purified genomic DNA. DNA concentration was estimated using a NanoDrop™ (Thermo Scientific Inc.).

Genomic DNA extraction from tissue cuts. First, OCT (optimum cutting temperature) compound was removed by washing once the tissue cuts with PBS 1×. Then, 400 μL of lysis buffer was applied onto the sample. The mixture was placed into a 1.5 mL eppendorf tube and supplemented with 10 μL of Proteinase K (20 mg/mL) followed by incubation at 56° C. for 1 h. Suspension was brought to 500 μL using distilled water. Then one volume of phenol-chloroform was added and following centrifugation, the aqueous phase was recovered in a new microfuge tube. Then DNA was precipitated using 1/10 volume of NaCl 5 M and two volumes of 100% ethanol followed by 5 min centrifugation ate 13200 rpm. The pellet was washed with 70% ethanol, centrifuged and the DNA was resuspended in double-distilled water. The genomic DNA concentration was assayed with a Nanodrop™ spectrophotometer (Thermo Scientific, Logan, Utah).

Immunohistochemistry for the identification of the dystrophin expression in muscle fibers. First, to withdraw the OCT compound we washed the slides three times for 5 min using PBS 1× to remove traces of OCT compound.

Then muscle sections were blocked for 1 h using PBS1× and 10% of FBS. Immediately, blocking solution was replaced by a solution containing the primary antibody mouse anti-dystrophin (NCL-Dys2, Novocastra) diluted 1:50 into PBS 1× and 10% FBS. The primary antibody was incubated for 1 h. Then slides were wash three times for 10 min using PBS1×. The secondary antibody goat anti-mouse Alexa Fluor 546. To prevent the sample from drying, slides were covered with 50% glycerol in PBS then covered with a cover slide. Fluorescence were observed using. Pictures were taken using a Nikon ISO 3200, with an exposure time of for ¼ sec.

PCR analysis of hybrid exons formed in the hDMD/mdx mouse model. Genomic DNA, extracted from hDMD muscles, was used to amplify the hybrid exon using a C1000 Touch thermocycler and primers Fw-Int50 (5′-TGCCTGGAGAAAGGGTTTTTGT-3′, SEQ ID NO: 222) and Rv-Ex54 (5′-TATCAGATAACAGGTAAGGCAGTG-3′, SEQ ID NO: 177). Analysis was then made by migration on 1.5% agarose gel in 1×TBE buffer containing Red Safe 1× dye (Chembio Inc.).

Cloning and Sequencing for the Hybrid Exon. The hybrid exon amplicons were purified using a gel extraction kit (Gel Extraction Kit GeneJET, Thermo Scientific Inc.). PCR purified fragments obtained were then dosed using a NanoDrop™ (Thermo Scientific Inc.). 15 ng of the PCR product was cloned into the plasmid vector pMiniT of the PCR Cloning Kit (NEB Inc. Ipswich, Mass., USA). Ligations were then transformed into competent bacteria provided by the manufacturer, and then seeded into LB Agar plates containing ampicillin (50 μg/mL). After 16 h incubation at 37° C., clones were taken and inoculated into 5 mL of LB medium (lysogeny broth) with ampicillin (50 μg/mL). The samples were incubated 16 h at 37° C. Bacterial cultures were used for plasmid DNA extraction using a Miniprep kit (GeneJET Plasmid Miniprep Kit, Thermo Scientific Inc.). The plasmids were dosed with a NanoDrop™ (Thermo Scientific Inc.) and then sent to the sequencing platform of the CHUL Research Centre (CHUQ). The sequences obtained were analyzed using the BLAST homology platform (NCBI, http://blast.ncbi.nlm.nih.gov/Blast.cgi).

### Example 9

### Analysis of In Vitro Cleavage by the SpCAS9/RNA Complex

SgRNAs 1-50 and 5-50, (see Table 3 above), were produced by in vitro transcription. Commercially produced crRNA and tracrRNA were also used in some experiments. The sgRNAs and crRNA targeted exon 50 preceding the deletion and exon 54 that follows the deletion causing the disease. The gRNA-targeted sequences were chosen to not only correct the reading frame but also the dystrophin protein structure. The cutting efficacy of the gRNAs and of the crRNA in complex with the Cas9 protein was initially tested on a PCR amplicon of the targeted region in exon 54. Results showed high cutting efficiency in vitro, since complete digestions were obtained in only 30 minutes of incubation at 37° C. (FIG. 8A).

### Example 10

### Surveyor Test on Exon 54 of the DMD Gene, Following Transduction of the CRISPR/CAS9 System with a Feldan Shuttle in Hela Cells

The SpCas9:crRNA:tracrRNA or the SpCas9:sgRNA complexes were then transduced in Hela cells using the Feldan Shuttle technology (FIG. 8B). Feldan Shuttle A (FSA) and Feldan Shuttle B (FSB) are polypeptide-based shuttle agents, which were kindly provided by Feldan Therapeutics Inc. Our experiments showed efficient cutting of the dystrophin gene using the Surveyor assay on the genomic extracted DNA only when the SpCas9 protein was delivered with the Feldan Shuttle in complex with the sgRNAs or with the crRNA:tracrRNA in cells (FIG. 8B). There was no difference in the cutting efficiency obtained using the sgRNA or the crRNA:tracrRNA. This shows that the use of sgRNA transcribed in vitro does not interfere with the activity of the Cas9 protein.

### Example 11

### Delivery of the SpCAS9: sgRNA Complex in the hDMD/mdx Mouse Model

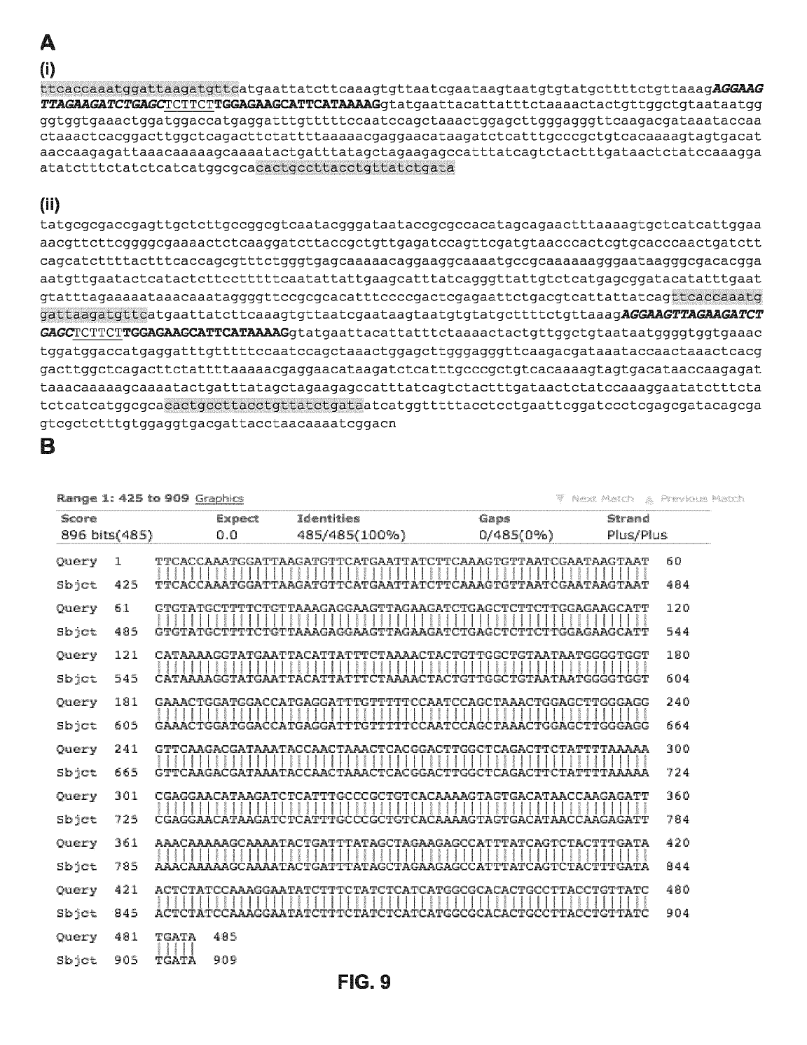

Following positive in vitro results, this gene editing approach was tested in vivo in the hDMD/mdx mouse model. This mouse model contains the complete human DMD gene integrated in its genome ('t Hoen et al., 2008). This allows us to verify whether the sgRNAs targeting the human DMD exons 50 and 54 were also able to induce specific deletions in vivo following delivery with different methods, i.e., a single injection of the SpCas9 protein in complex with the 2 sgRNAs was made into the Tibialis anterior (TA) of the mouse. This complex was transduced in the muscle fibers of the TA either by electroporation, lipofection (Lipofectamine™ RNAimax) or with two different Feldan Shuttles. The mice were sacrificed 72 hours later and the treated muscle was extracted and divided in 4 equivalent parts to analyze the distribution of the gene editing. Genomic DNA was extracted for PCR amplification and sequencing. The region between exons 50 and 54 is about 160 kb, and thus cannot be amplified by conventional PCR. However, if the hybrid exon is formed following DSBs in both targeted exons, a PCR fragment of about 600 bp can be observed (FIG. 8C). The amplicons obtained following transduction of the sgRNA pair in complex with the SpCas9 protein were gel purified and cloned into the pMiniT plasmid, transformed in bacteria and clones were screened for successful insertions. Positive clones, according to the digestion pattern (data not shown), were sent for sequencing to demonstrate the presence of a hybrid exon formed by the fusion of a part of exon 50 with a portion of exon 54 (FIG. 9A(i)). The expected hybrid exon 50-54 was present (FIG. 9A(ii), SEQ ID NO: 200), as indicated by the perfect alignment between the observed sequence and the expected sequence (FIG. 9b). This result was obtained for about 60% of the clone sequences. The other 40% of the sequences contained INDELs at the junction site. These INDELs are made in the correction of the gene by NHEJ, which is also observed by our group when the same hybrid exon was formed using plasmids encoding the SpCas9 gene and the same sgRNAs used in our experiment.

### Example 12

### Strategy for the Identification of gRNAs of Interest Targeting the Human Dystrophin Gene

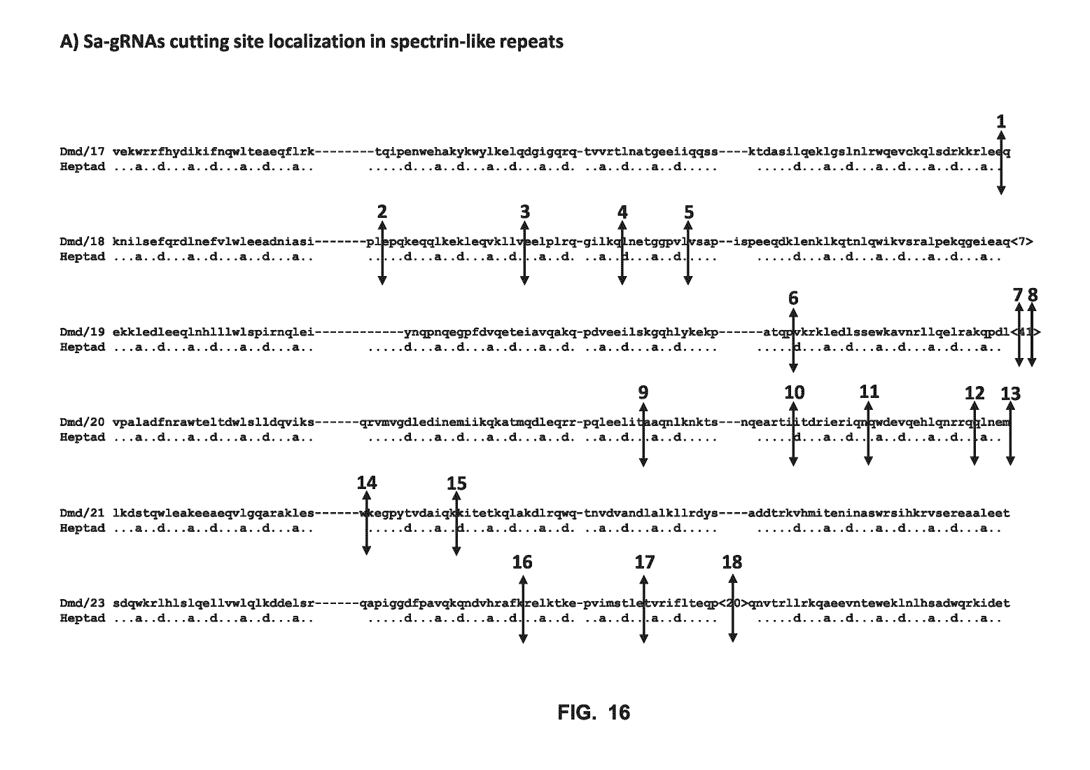

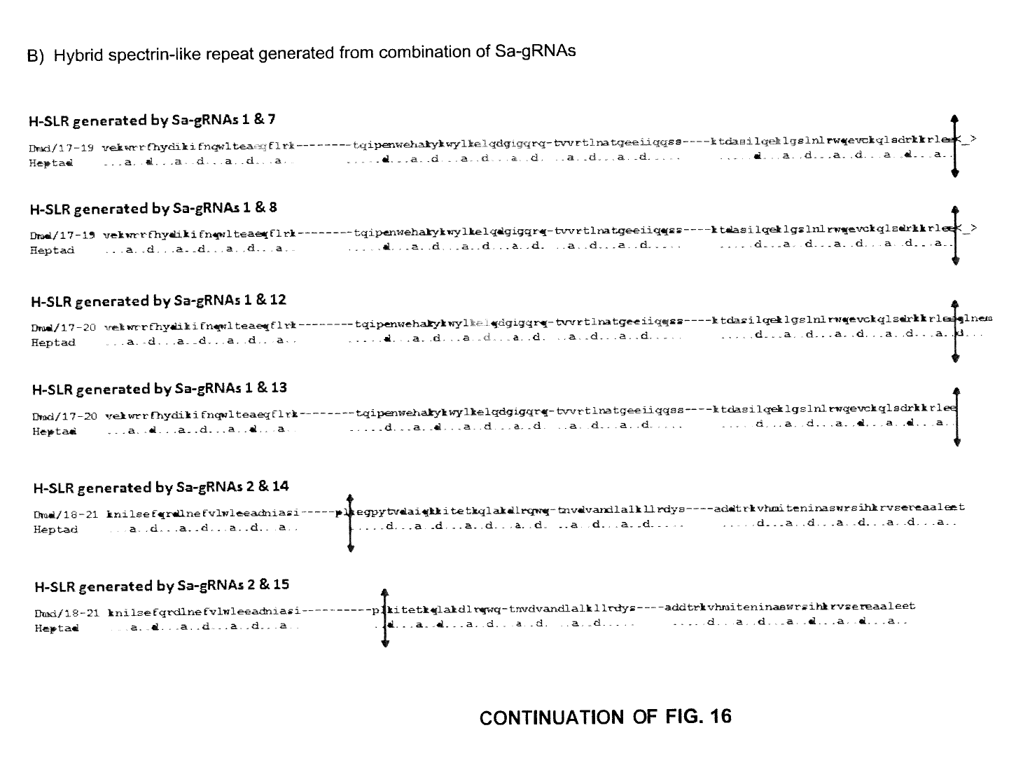

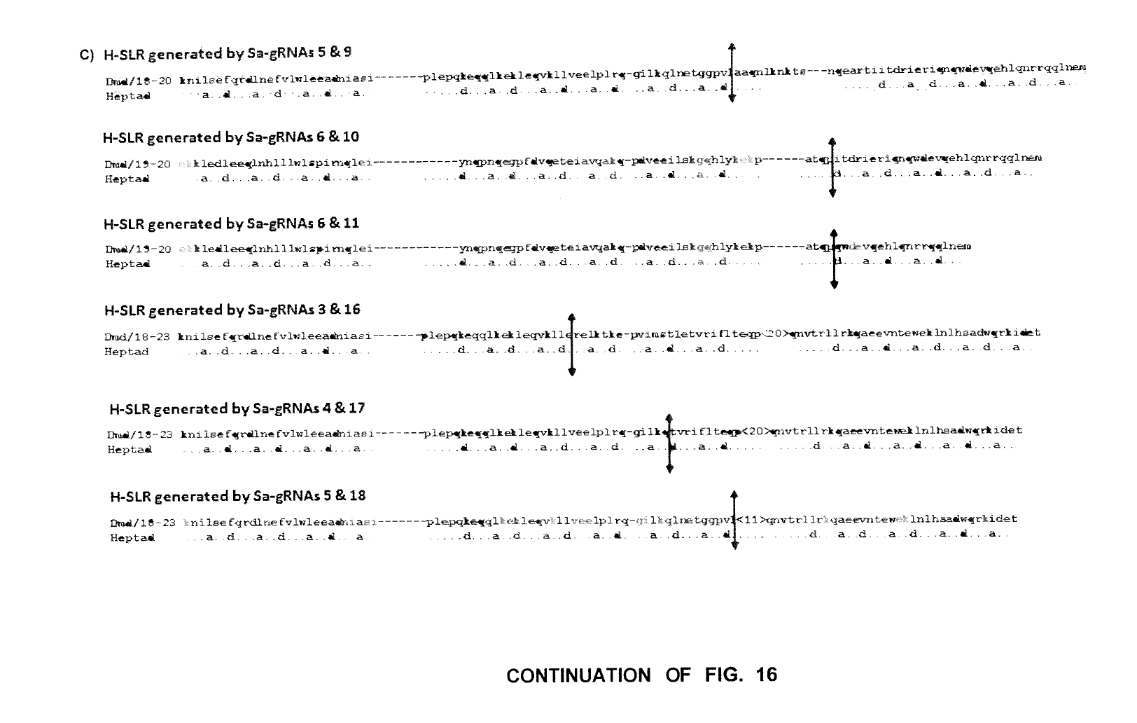

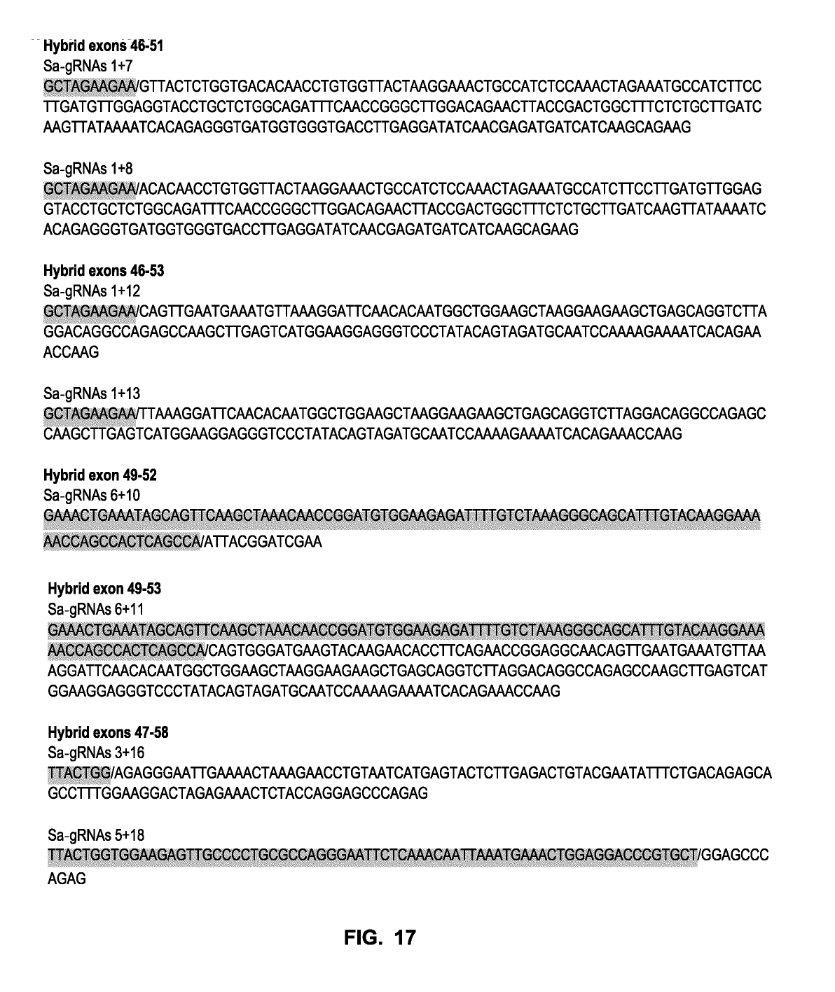

Our primary goal was to establish a strategy based on the creation of a hybrid exon allowing the correction of the dystrophin gene reading frame, in the case of DMD patients affected by the deletion of exon 50. Thus, we screened all possible gRNAs target sites surrounding exon 50. We identified all sites from exon 46 to exon 58. We identified nearly 50 gRNAs that can be used with the Cas9 protein from Staphylococcus aureus (S. aureus). As SaCas9 nuclease induces a double strand break (DSB) precisely 3 nucleotides upstream of the PAM (NNGRRT), we were able to select gRNAs that can be combined to create a new hybrid exon that restores a normal reading frame in the dystrophin gene. This hybrid junction could permit the production of an internally-truncated dystrophin. We focused on combinations of gRNAs where the hybrid junction maintains the configuration of a normal spectrin-like repeat, where hydrophobic amino acids are localized in position “a” and “d” of the heptad motif (spectrin-like repeats are composed of α-helixes each containing 7 amino-acids (a to f) where hydrophobic amino acid are in the location “a” and “d”; see for example FIG. 16). In order to assess the correct localization of those amino acids, we referred to the eDystrophin database which provides information about the structural domains of the dystrophin protein (http://edystrophin.genouest.org/). As a result of our preliminary analysis, we focused our work on 18 gRNAs (see Table 6) that generate 12 hybrid exons not only correcting the reading frame of the dystrophin gene but also maintaining the structure of spectrin-like repeats.

### Example 13

### Testing of Individual gRNAs for the saCas9/CRISPR System

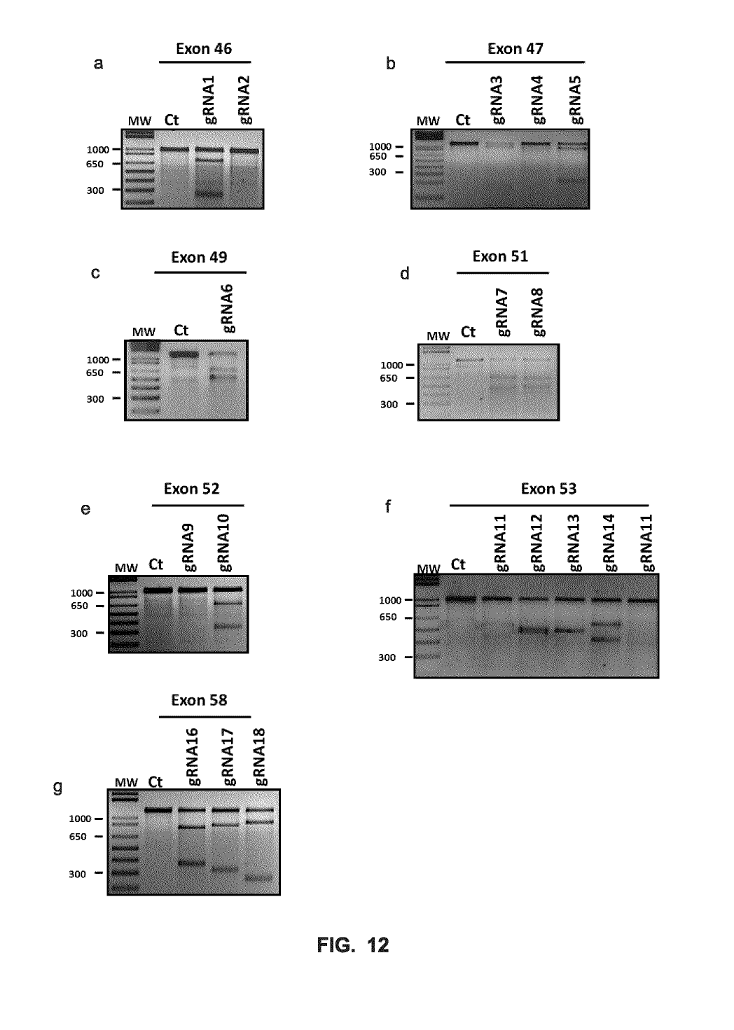

We designed 18 gRNAs that we cloned into the PX601-AAV-CMV::NLS-SaCas9-NLS-3xHA-bGHpA;U6::Bsal-sgRNA plasmid (Addgene #61591) (Table 6). In order to assess the activity of these gRNAs, we transfected 293T cells. 48 hours post transfection we extracted genomic DNA. Targeted exons 46, 47, 49, 51, 52, 53, 58 were amplified by PCR then submitted to a “Surveyor” enzyme assay for the detection of INDELs (FIG. 12). Among the 18 gRNAs, gRNA 2, gRNA 4, gRNA 9, gRNA 11, gRNA 15 show few or no activity whereas all remaining gRNAs exhibited good cleavage efficiency and specificity as they generated cleave bands at the expected size.

### Example 14

### Testing of gRNA Pairs Using the saCas9/CRISPR System in 293T Cells

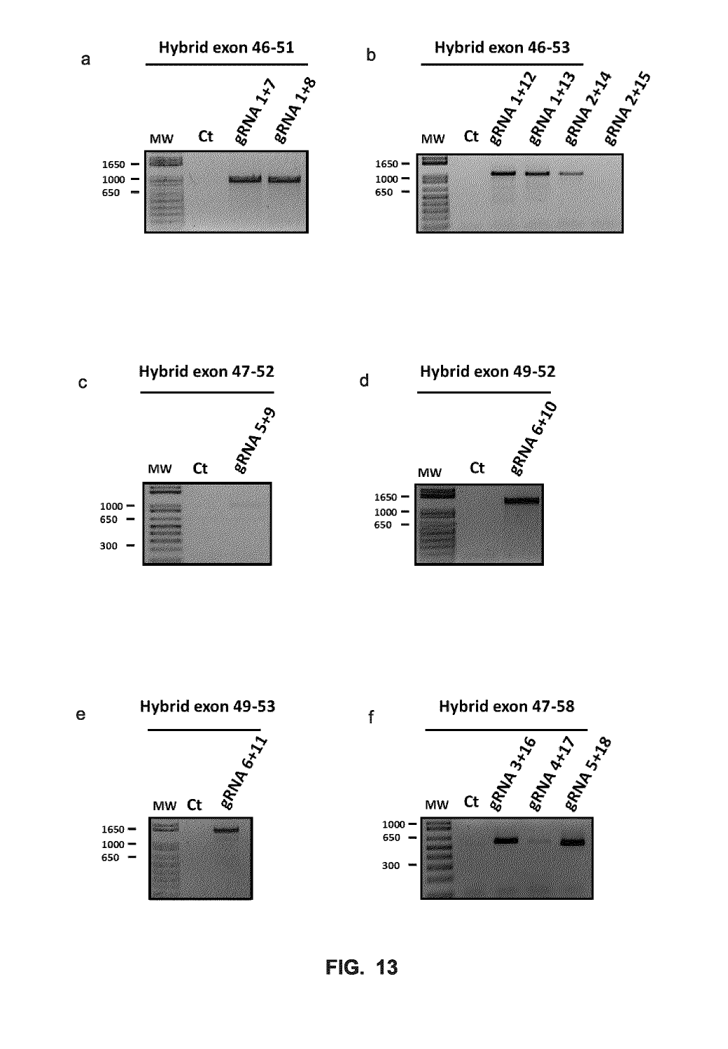

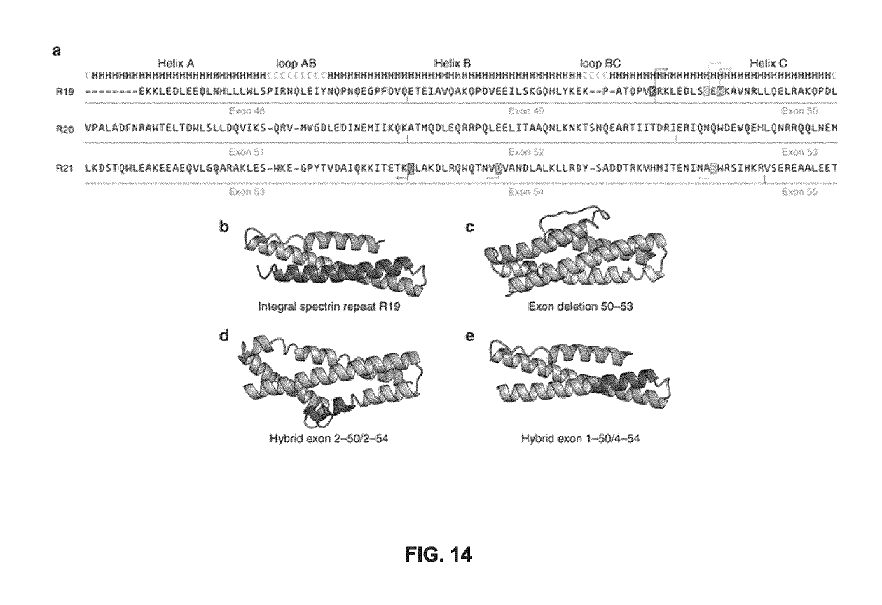

293T cells were co-transfected with combinations (see Table 7) of gRNAs that might allow to create large genomic deletion and precisely connect exons, surrounding a deletion of the exon 50, to the right nucleotides. Thus, we tested the 12 combinations of gRNAs we identified. Hybrid exons were identified by PCR using the forward primer for one of the targeted exon and a reverse primer for the other, previously used when gRNAs were individually tested (FIG. 13). Thus, PCR amplification is only possible if a deletion occurred between the two targeted exons as there are too spaced by the introns.

### Example 15

### Characterization of Hybrid Exons in 293T Cells (saCas9/CRISPR System)

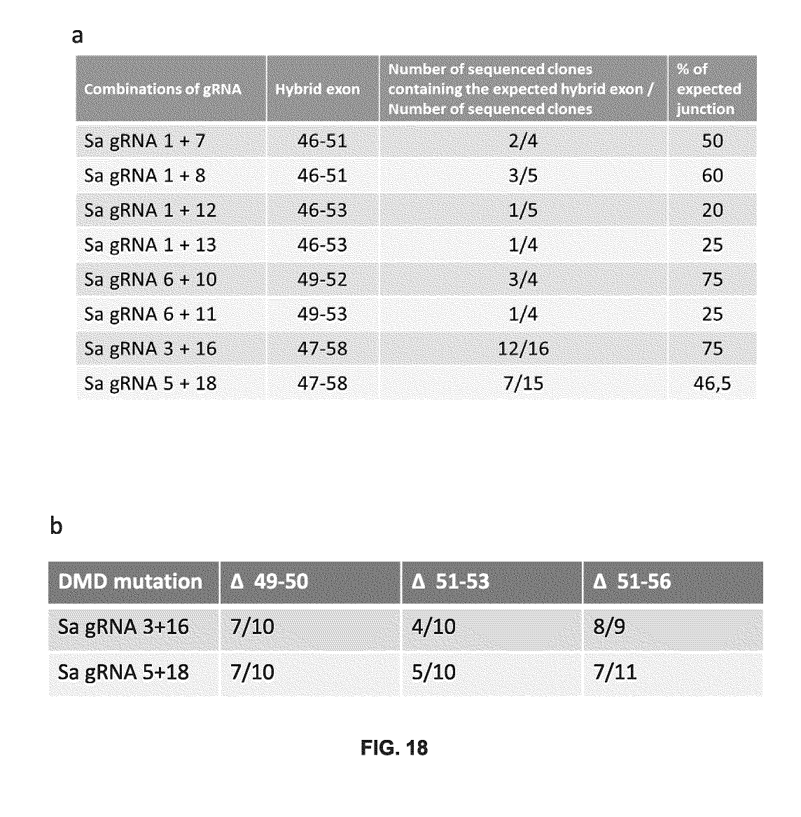

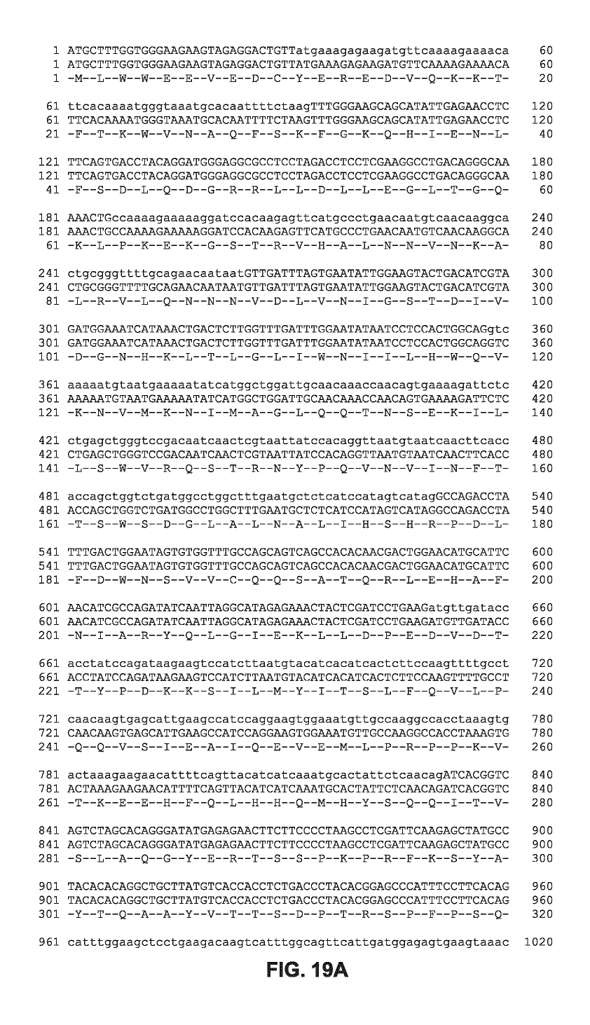

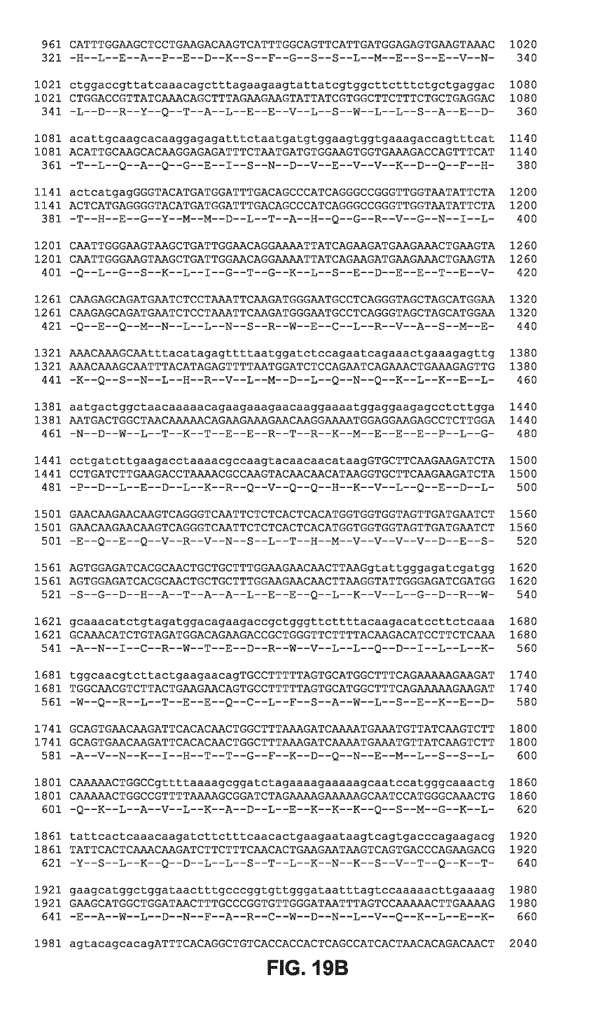

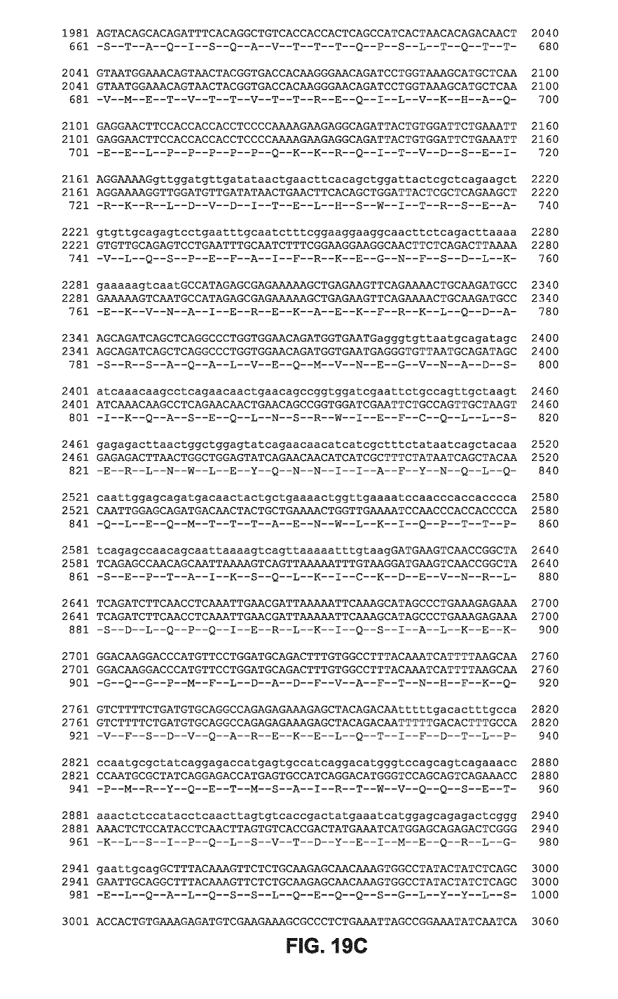

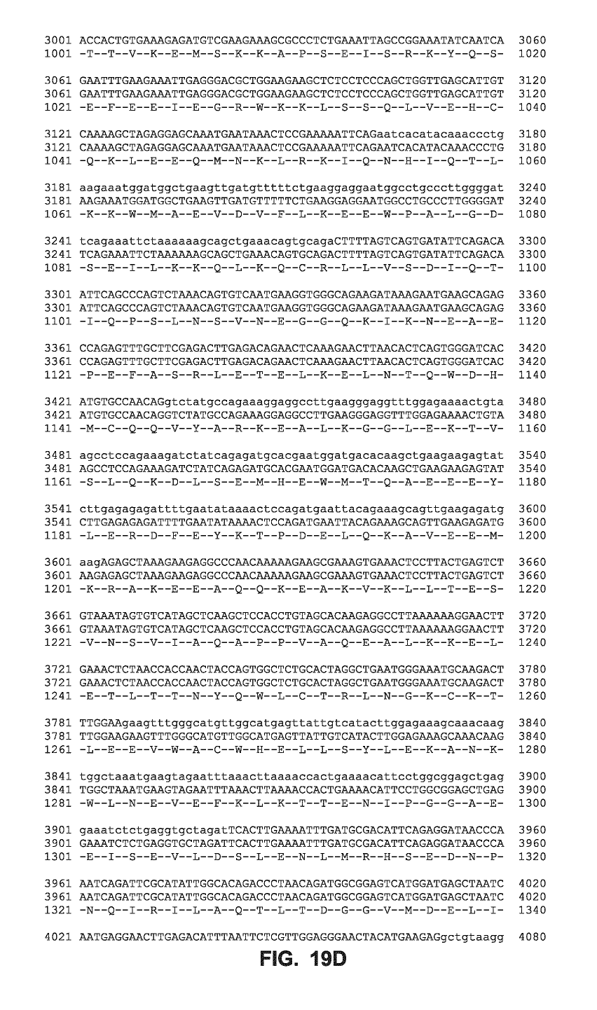

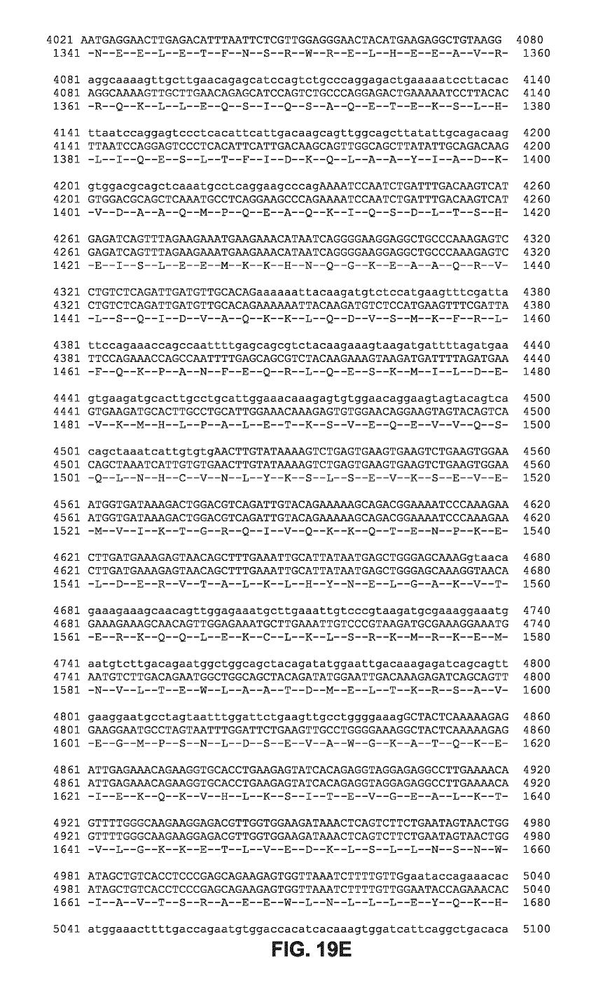

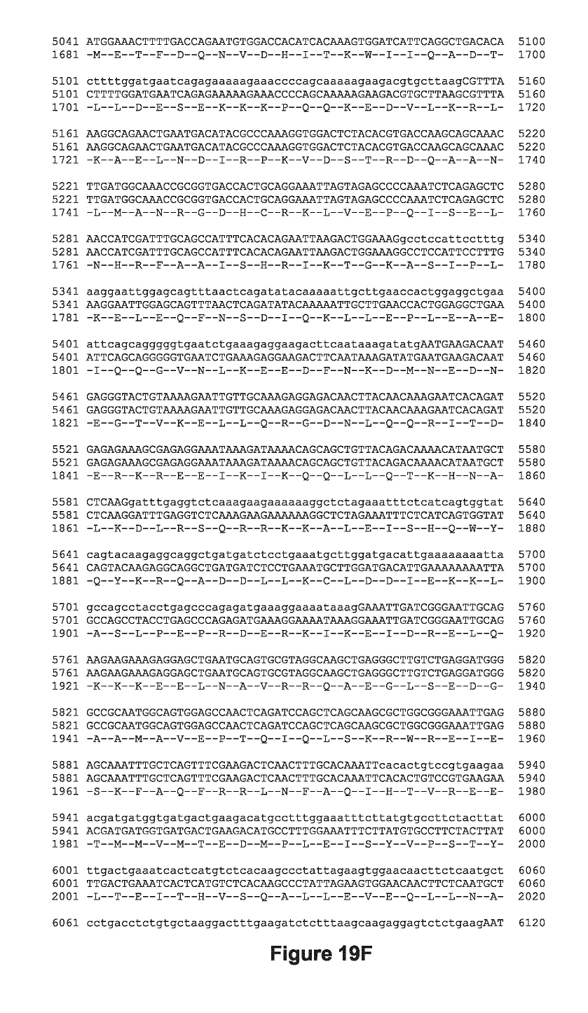

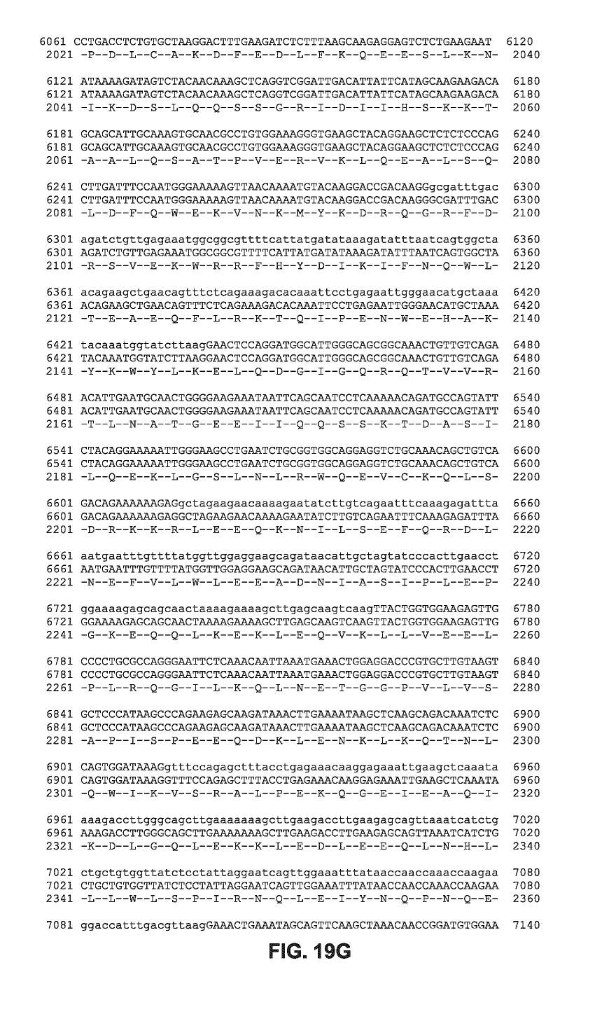

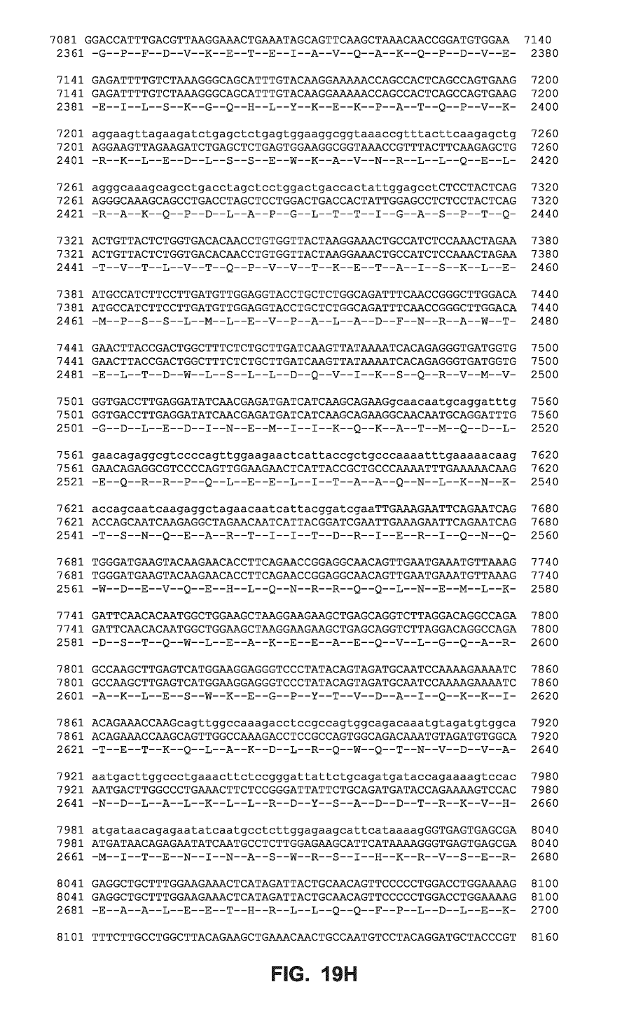

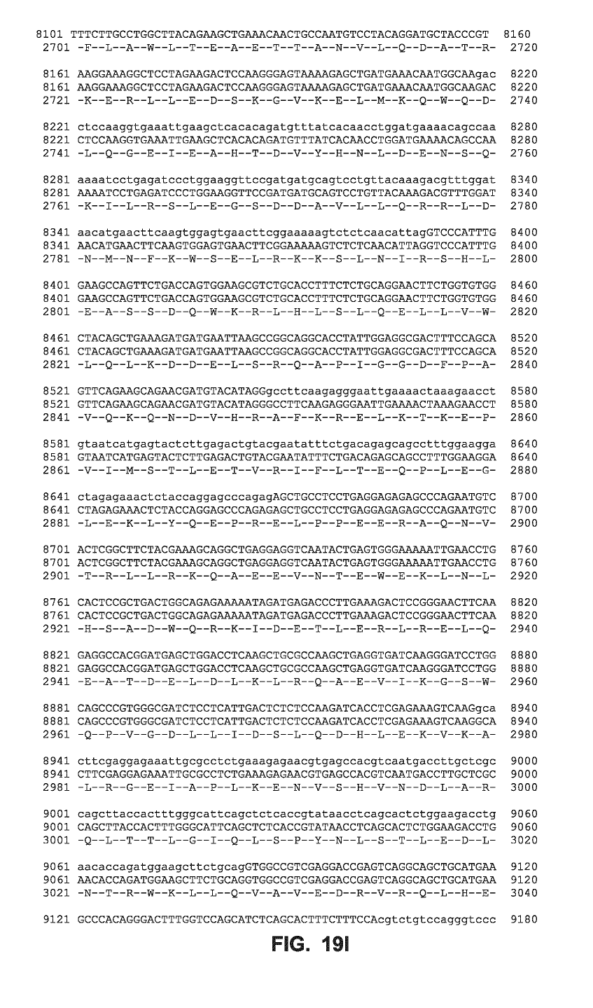

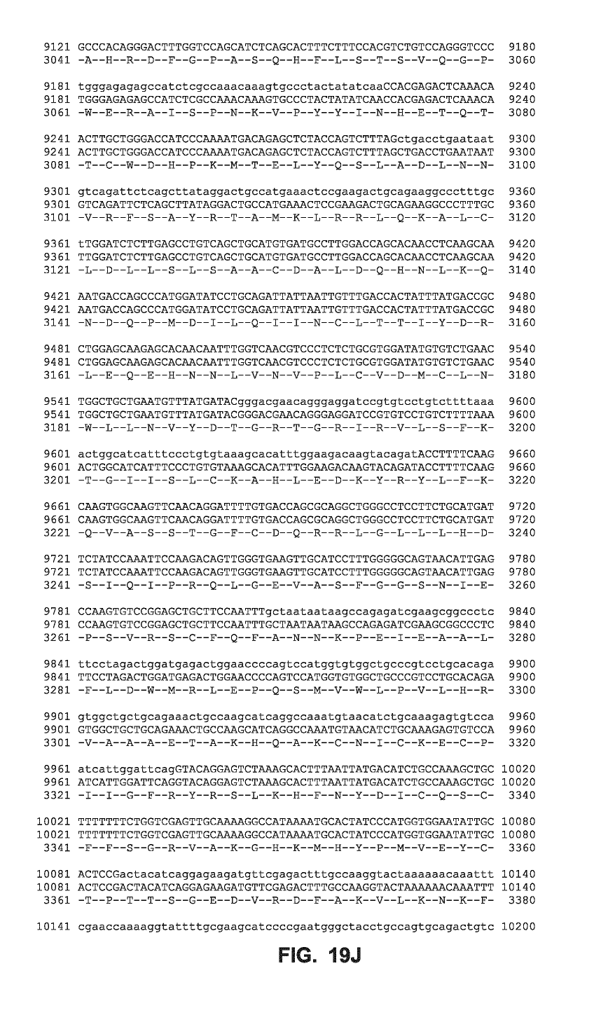

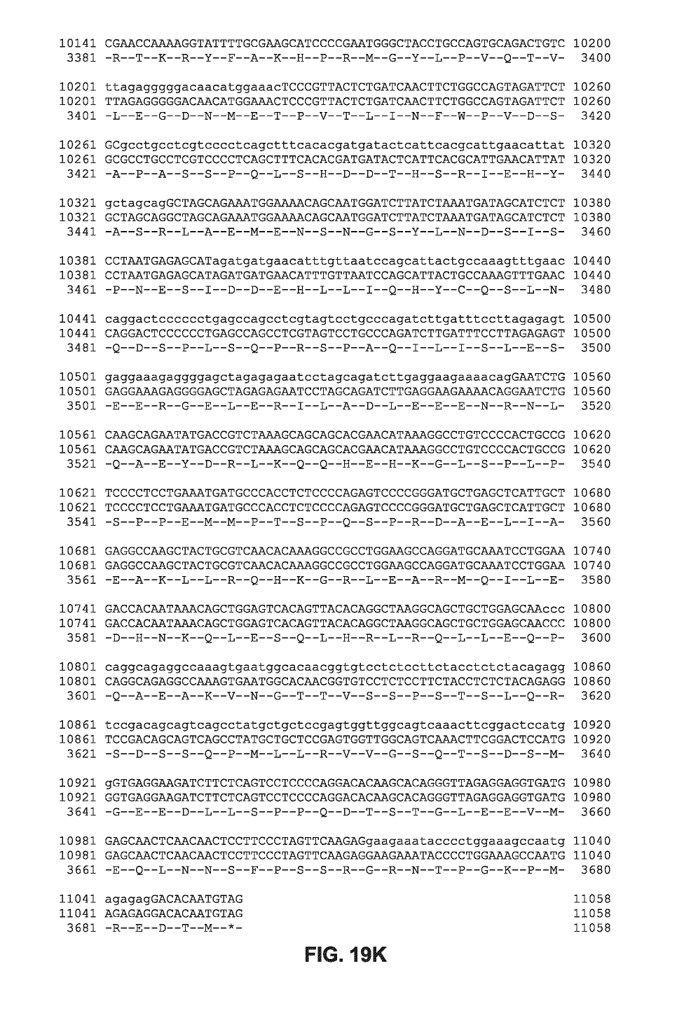

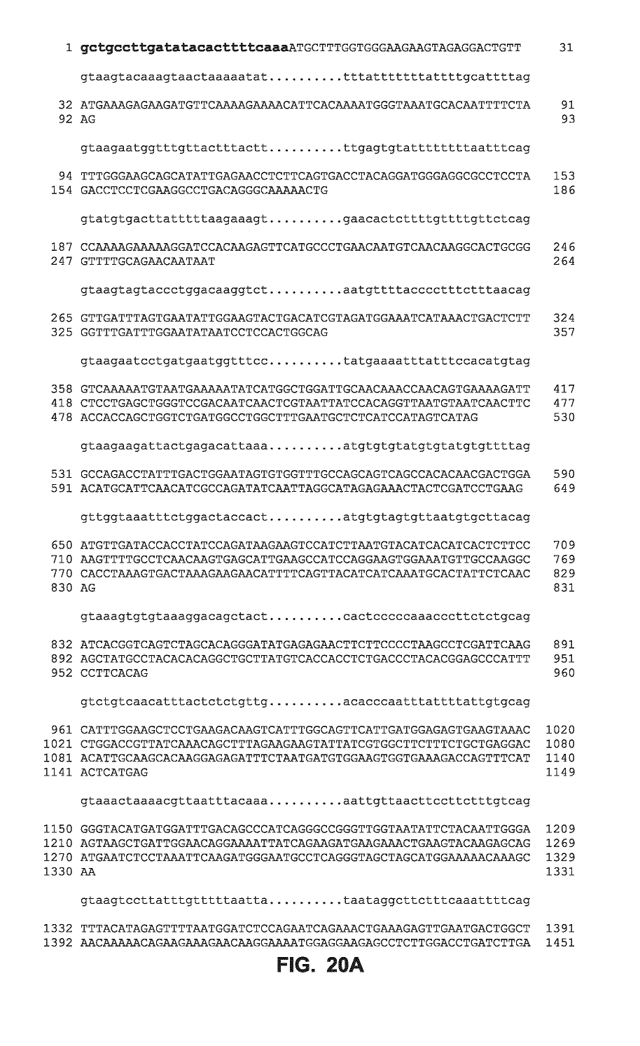

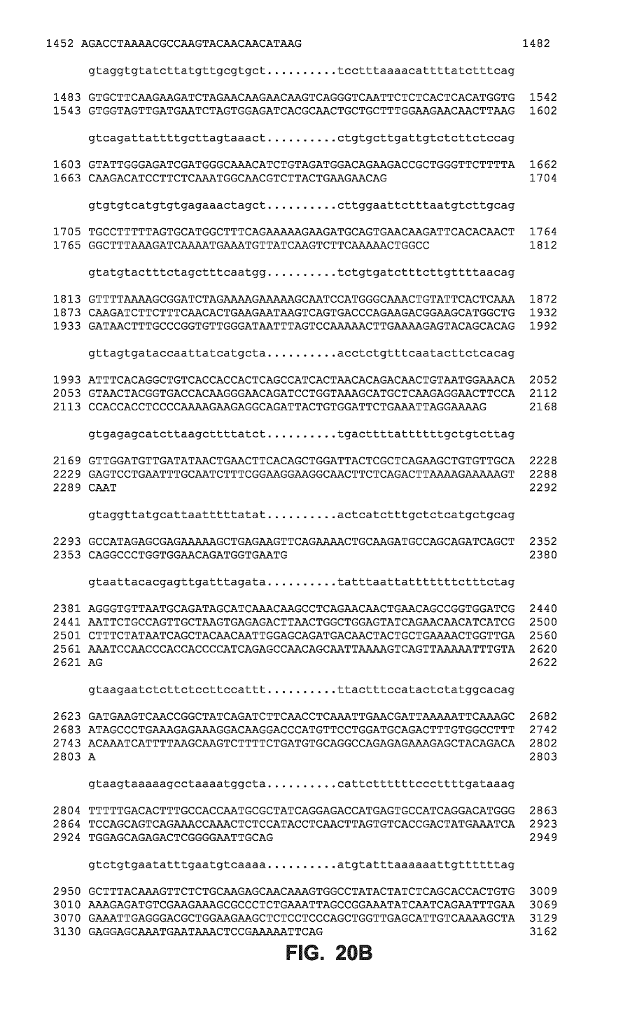

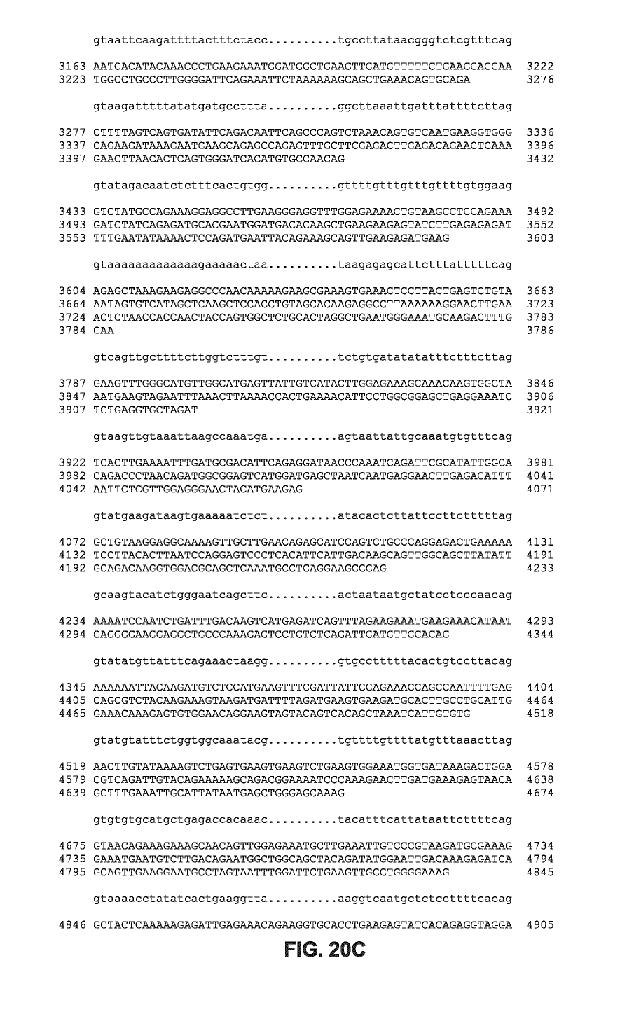

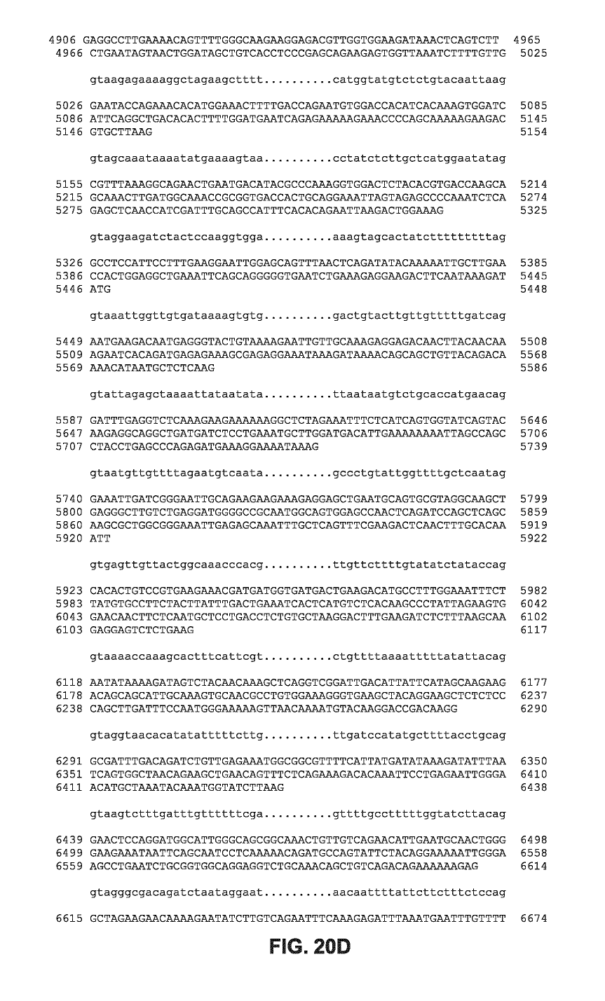

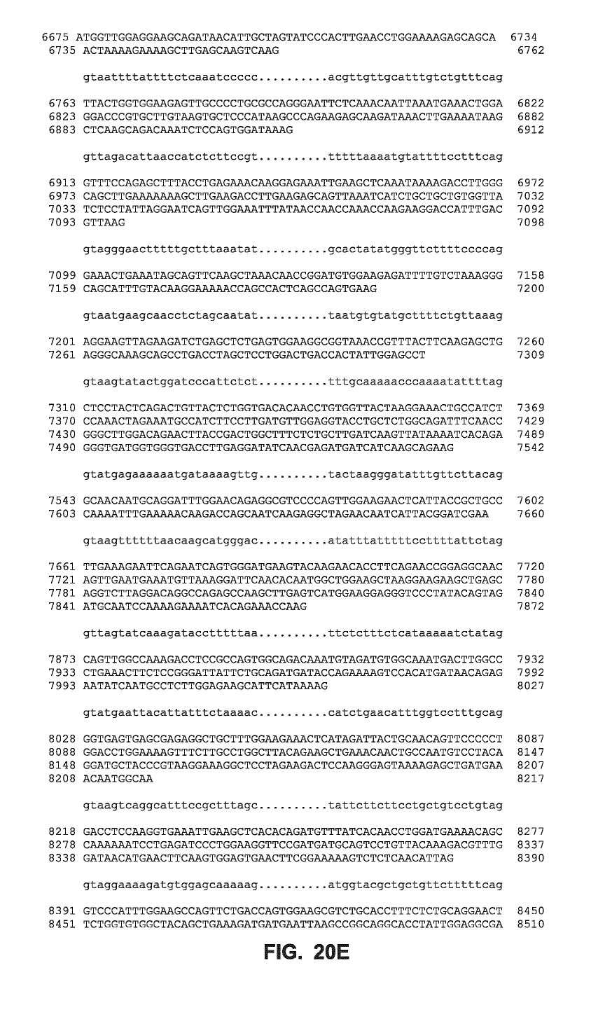

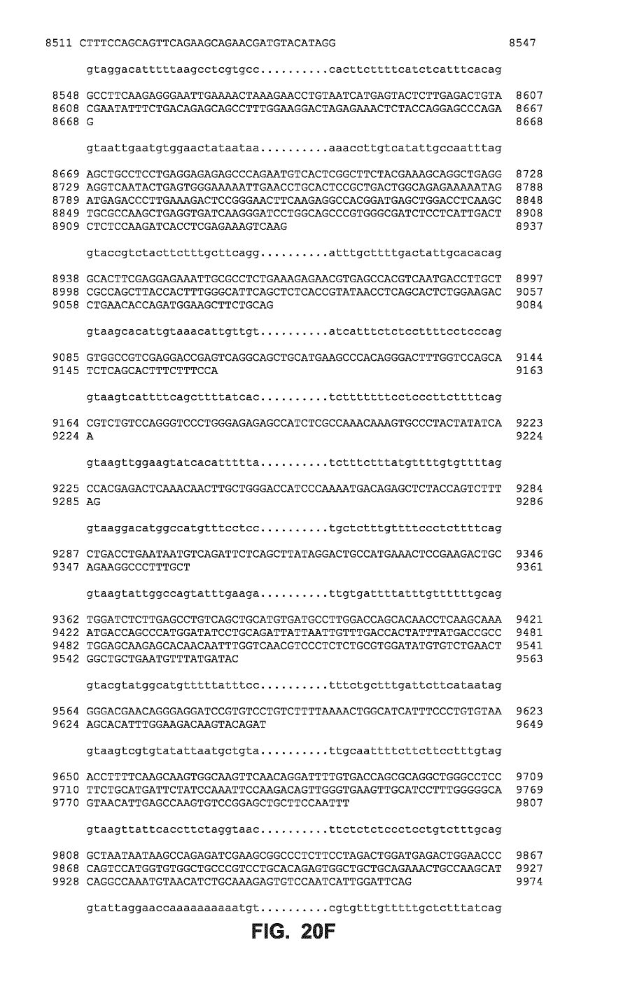

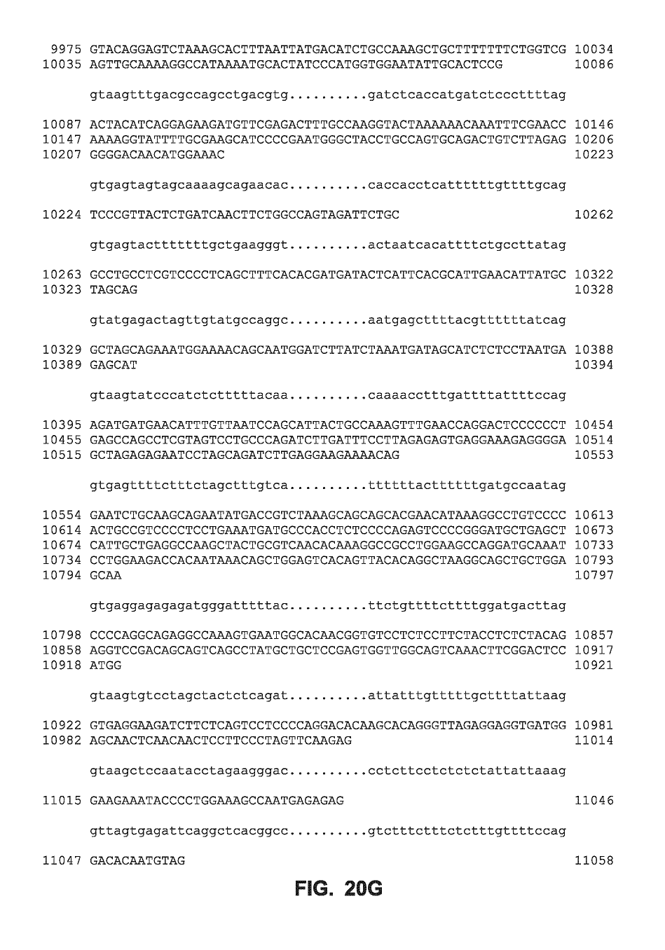

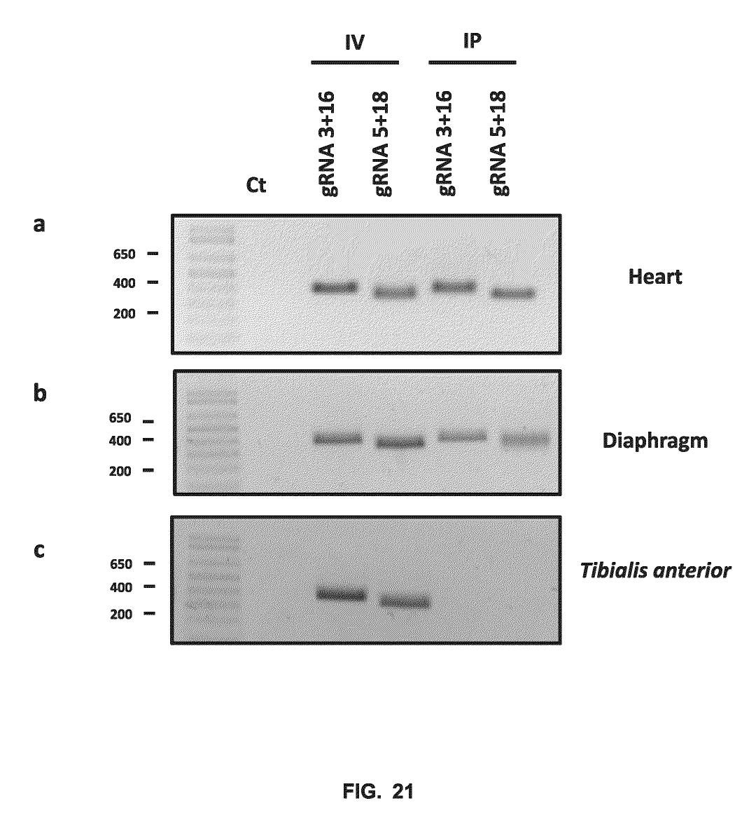

PCR products from amplification of hybrid exons were purified then cloned into a pMiniT vector for the sequencing of the hybrid exons resulting from large genomic deletion by the SaCas9 and 2 gRNAs surrounding the exon 50 deletion (FIG. 18a). Finally, combinations of gRNAs 3 and 16 and gRNAs 5 and 18 seemed the most promising as they allow to create the largest genomic deletion thus permitting to cover up to 40% of mutations identified in DMD patients. In addition, the combination of gRNAs 3 and 16 creating a 450 kbp deletion is the most precise combination as the junction is the expected one in 75% of the tested clones.

### Example 16

### Dystrophin Exon Targeting in DMD Myoblasts Using the saCas9/CRISPR System

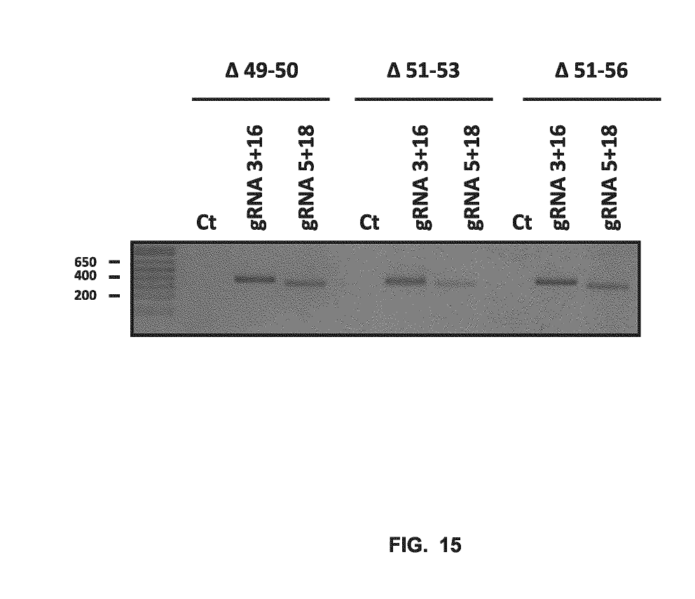

Myoblast cells from DMD patients were co-transfected with two combinations of gRNAs, combination of gRNAs 3 and 16 and the combination of gRNAs 5 and 18, that might allow to create large genomic deletion and precisely connect exons 47 and 58. These combinations should be able to create a hybrid exon with a correct reading frame leading to the production of a dystrophin protein with a hybrid spectrin like repeat. Hybrid exons 47-58 were identified by PCR using the primers forward Sense 47′ (SEQ ID NO: 194) and the reverse Antisense 58′ (SEQ ID NO: 195) (FIG. 15). Thus, PCR amplification is only possible if a deletion occurred between the two targeted exons as there are too spaced by the introns.

### Example 17

### Characterization of the Hybrid Exon 47-58 in Myoblasts (saCas9 CRISPR System)

PCR products from amplification of the hybrid exons 47-58 were purified then cloned into a pMiniT vector for the sequencing of the hybrid exons resulting from large deletion by SaCas9 and 2 gRNAs surrounding exon 50 deletion (FIG. 18b).

### Example 18

### In Vivo Correction in the HDMD/MDX Mouse (saCas9 CRISPR System)

Following the identification and characterization of pairs of gRNAs in 293T then in myoblasts from different DMD patients, we tested the viral AAV-mediated delivery of the CRISPR system from S. aureus and the pair of gRNAs 3 and 16 and the pair of gRNAs 5 and 18 using the AAV serotype 9. One AAV9 permitted the expression of the SaCas9 nuclease under the control of the CMV promotor while a second AAV9 coded for a pair of gRNAs (gRNAs 3 and 16 or gRNAs 5 and 18). We compared the administration of the viral particles through intraperitoneal and intravenous injection path into 6 week old hDMD/mdx mice. Mice were sacrificed 6 weeks after the injection. The tissues—heart, diaphragm, Tibilalis anterior (TA), brain/cerebellum, liver-were collected and incubated overnight in 30% sucrose. After harvesting in OCT medium and liquid nitrogen freezing followed by processing of the tissue onto glass slides, genomic DNA was extracted and purified. For an optimal detection of the successful formation of the hybrid exon 47-58 in vivo we performed a nested-PCR. The first PCR was done using the forward primer sense 47 and the reverse primer antisense 58. The second PCR the forward Sense 47′ (SEQ ID NO: 194) and the reverse Antisense 58′ (SEQ ID NO: 195). FIG. 21 shows the detection of the formation of the hybrid exon 47-58 following nested-PCR with genomic DNA from the heart (a), the diaphragm (b), the TA (c). We observed that in the heart, as in the diaphragm, the viral delivery of the SaCas9 and of a pair of gRNAs, gRNAs 3 and 16 as well as gRNAs 5 and 18, permit the formation of the hybrid exon 47-58, in comparison to the non-treated mouse (NT). Besides, in the TA, the amplification of the hybrid exon 47-58 was detected when the SaCas9 and the pair of gRNAs 3 and 16 or the pair of gRNAs 5 and 18 were only delivered through intravenous injection.

### Example 19

### Methods for Delivery of the SpCAS9: sgRNA Complex in the RAG/MDX Dystrophic Mouse Model (Examples 20-23)

gRNAs cloning into the plasmid pSpCas9(BB)-2A-Puro. The oligonucleotides coding for the target sequences and their complementary sequences were ordered from IDT and cloned into the plasmid pSpCas9(BB)-2A-Puro (pX459) (Addgene plasmid #48139) containing two Bbsl restriction sites necessary for insertion of a protospacer under the control of the U6 promoter. Cloning was performed as previously described in Example 8. The pSpCas(BB)-2A-Puro plasmid contains the Cas9 of S. pyogenes, and a puromcyin resistance gene under the control of the CBh promoter; both genes are separated by a sequence encoding the self-cleavable peptide T2A. The puromycin resistance gene allows to select and enriched the transfected C2C12 cells (ATCC® CRL1772), a hard to transfect cell line.

The gRNA activities were tested individually or in pairs by transfection of the pSpCas9(BB)-2A-puromycin plasmid encoding each gRNA in C2C12 cells (murine myoblast). The C2C12 cells were grown in Dulbecco's modified Eagle medium (DMEM) medium (Invitrogen, Grand Island, N.Y.) containing 10% fetal bovine serum (FBS) and antibiotics (penicillin 100 U/ml/streptomycin 100 μg/ml). 24 hours after the transfection, cells were submitted to 2 μg/mL of puromycin for 48 hours to select transfected C2C12 cells. Following puromycin selection, cells were grown for 48 hours in the absence of puromycin.

Genomic DNA was extracted as described in Example 8.

PCR amplification of modified DYS gene. To assess the correct cuts and deletions the targeted region was amplified by PCR using the forward primer Fw2-i22 (5′-CTGTGATGTGAGGACATATAAAGAC 3′) (SEQ ID NO: 204) located in intron 22 and the reverse primer Rev1-i23 (5′-TCAATGTAGGGAAGGAAATATGGCA 3′) (SEQ ID NO: 205) located in intron 23. PCR amplifications was performed in a thermal cycler C1000 Touch of BIO RAD (Hercules, Calif.) with the Phusion™ high fidelity polymerase (Thermo scientific, EU, Lithuania). Exon 23 was amplified using the following program: 98° C./10 sec, 61.2° C./30 sec, 72° C./45 sec for 35 cycles. For the analysis of individual gRNA activities, PCR products were submitted to Surveyor enzyme assay. For the analysis of the pairs of gRNAs, PCR products were loaded onto an agarose gel. If a pair of gRNAs permits the deletion of exon 23, PCR amplification will allow the detection of a wild type band along with a shorten band corresponding to remaining parts of intron 22 and intron 23 following the deletion.

Amplicons generated for exon 23 were used to perform the Surveyor assay. There was first a hybridization step of the amplicons using a slow-hybridization program (denaturation at 95° C. for 5 min followed by gradual cooling of the amplicons) with BIO RAD thermal cycler C1000Touch™ (Hercules, Calif.). Subsequently, the amplicons were digested with nuclease Cel (Integrated DNA Technologies, Coralville, Iowa) in the thermal cycler at 42° C. for 1 hour. The digestion products were visualized on agarose gel 2%.

For in vivo experiments, we used the sgRNA 20, sgRNA 22 and sgRNA 23 that were ordered from Synthego (Redwood City, Calif.). We received 1 nmole of each sgRNA that were resuspended into nuclease and RNase free water to a stock concentration of 50 μM, harvested at −80° C. As a working solution, sgRNAs were diluted to 10 μM and stored at −20° C.

In vivo protein delivery. 2.5 μM of SpCas9 protein were complexed with 2 μM of sgRNAs and then incubated for 5 to 10 min at RT. The sgRNAs mixture is composed of 1 μM of one sgRNA targeting a region upstream of exon 23 and 1 μM of a sgRNA targeting a region downstream of exon 23. Resulting complexes were mixed with 3 different Feldan Shuttles, Feldan Shuttle A (FSA) at 50 μM or Feldan Shuttle B (FSB) at 35 μM or Feldan Shuttle C (FSC) at 50 μM. In each condition, the final volume of the reaction was maintained at 40 μL. For the negative control, SpCas9 complexed with sgRNAs were injected without Feldan Shuttle. The tested solutions were injected longitudinally in the Tibialis anterior muscles. The mice were sacrificed 3 weeks later and the muscles were collected. The muscles were incubated overnight at 4° C. in 30% of sucrose. Then muscles were embedded into OCT medium and flash frozen in liquid nitrogen. The tissues were then processed using a cryostat (Leica system) allowing to make 16 μm transversal cross sections onto glycined glass slides.

Strategy for the PCR amplification of hybrid introns. For the detection of the deletion of exon 23 we performed an optimized nested-PCR approach. In the first PCR reaction, we used the external forward primer F1-i22 (5′-GAACATGTCTTATCAGTCAAGAGATC) (SEQ ID NO: 223) and the external reverse primer Rev1-i23 (5′-TCAATGTAGGGAAGGAAATATGGCA) (SEQ ID NO: 224) along with a forward “poison” primer Fw-P (5′-AACTATCTGAGTGACACTGTGAAGG) (SEQ ID NO: 225) targeting exon 23. This strategy allows to mainly amplify the wild-type region generated from the forward “poison” primer and the external reverse primer. However, if exon 23 is deleted, the forward poison primer Fw-P is not able to generate an amplification product. Thus, from genomic DNA, the two external primers will amplify and enriched genomic DNA where exon 23 is deleted, resulting in amplification of a hybrid intron formed by the remaining part of intron 22 and a part of intron 23. Following the first PCR, nested-PCR is performed using the internal forward primer F2-i22 (5′-CTGTGATGTGAGGACATATAAAGAC) (SEQ ID NO: 226) and the internal reverse primer Rev2-i23 (5′-CAGACAATCCAAGAAGGTATGACAC) (SEQ ID NO: 227). These two internal primers will amplify from the previous amplicons generated with the external primers only; i.e., amplicons lacking exon 23. This combination of internal primers only permits amplification from the amplicons obtained with the two external primers F1-i22 and Rev1-i23.

Hybrid intron analysis. The amplicons of hybrid introns obtained by the amplification of genomic DNA extracted from TA muscle were cloned into the linearized cloning vector pMiniT™ (NEB, Ipwisch, MA). Then, plasmid DNA was extracted with the Miniprep™ Kit (Thermo Scientific, EU, Lithuania). The clones were sent for sequencing using primers provided by the manufacturer (NEB, Ipwisch, MA). Sequencing results were analyzed with the CLC Sequence Viewer software (CLCBio).

Dystrophin positive fibers count. We counted the number of dystrophin positive fibers in three successive muscle cuts exhibiting the highest number of positive and established means and standard deviation.

### Example 20

### gRNAs Targeting Intron 22 and Intron 23 to Remove Mutated Exon 23 in the Mouse Model Rag/Mdx

In the mouse model Rag/mdx the dystrophin gene is mutated in exon 23. This nonsense point mutation C572T, thus creates a premature STOP codon and abrogates the synthesis of the 427 kDa wild type dystrophin protein. As the number of nucleotides in exon 23 is a multiple of three, its deletion should allow correcting the dystrophin gene reading frame. Consequently, we aimed at deleting exon 23 by designing a pair of gRNAs that targets its surroundings introns. Thus, we identified 4 gRNAs target sites upstream of exon 23, in intron 22 and 4 gRNAs target sites downstream of exon 23, in intron 23 (see Table 8). Two target sites span the exon 23/intron 23 junction but the cut which is introduced into the gene in located within the intronic sequence (see gRNA #20 and 26 in Table 8).

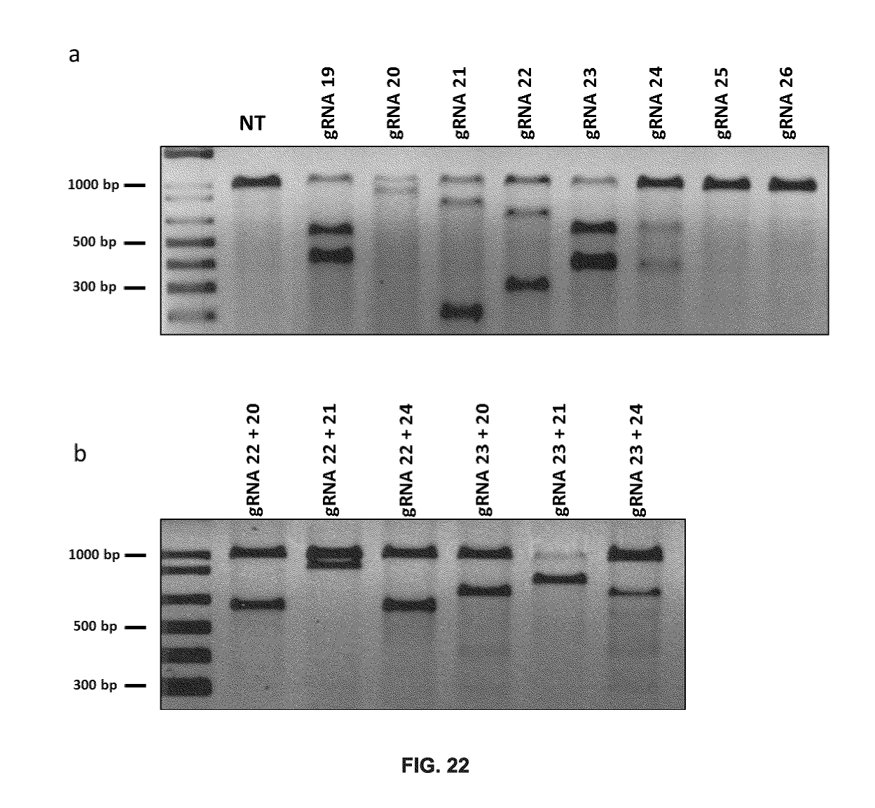

We thus prepared 8 gRNAs expression construct targeting intron 22 or intron 23 that we cloned into PX459. In order to assess the activity of these 8 gRNAs, C2C12 cells were transfected. 24 hours post transfection we selected transfected cells using 1 μg/mL of puromycin for 48 hours. Following another 48 hours of culture in selection free medium, we extracted genomic DNA. We amplified by PCR exon 23 and its surrounding introns and submitted the samples to a “Surveyor” detection assay to assess the creation of INDELs (FIG. 22a). Among the 8 gRNAs we tested, gRNA 19, gRNA 20, gRNA 21, gRNA 22, gRNA 23 and gRNA 24 demonstrated good cleavage efficiency whereas no activity was detected for gRNA 25 and gRNA 26 under the conditions tested.

### Example 21

### Testing of gRNAs Pairs for the Deletion of Exon 23 in C2C12 Cells

Based on the gRNAs identified in Example 20, we tested combinations of gRNAs that could delete exon 23. Thus, we tested the combination of gRNAs 22 and 20, gRNAs 22 and 21, gRNAs 22 and 24, gRNAs 23 and 20, gRNAs 23 and 21 and gRNAs 23 and 24. To assay the deletion of exon 23, PCR products were amplified from purified genomic DNA using the primers Fw2-i22 and Rev1-i23 and loaded onto an agarose gel (FIG. 22b). In each condition, PCR reaction allowed the detection of the wild type band formed by the complete intron 22, exon 23 and intron 23. Besides for each gRNAs combination that were tested, the amplification permits to detect a second PCR product which size is smaller than the wild type product. These truncated products correspond to the sizes resulting from the deletion of the exon 23. For further in vivo experiments, we choose to focus on the combination of gRNAs 20 and 22 and the combination of gRNA 20 and 23.

### Example 22

### Testing gRNAs Pairs for the Deletion of Mutated Exon 23 in the Mouse Model Rag/Mdx

For the in vivo experiments, we used a non-viral delivery approach based on a peptide-mediated delivery of the Cas9 protein along with a pair of gRNAs that target DNA sequences upstream and downstream of mutated exon 23 in the dystrophic mouse model Rag/mdx. This non-viral delivery tool, known as the Feldan Shuttle (FS), permits to bind the ribonucleoprotein complex Cas9/gRNA through non-covalent binding. Upon cell entry, pH variation leads to the disruption of the interaction between the FS and the ribonucleoprotein complex. Three shuttles from Feldan Therapeutics were tested: FSA, FSB and FSC.

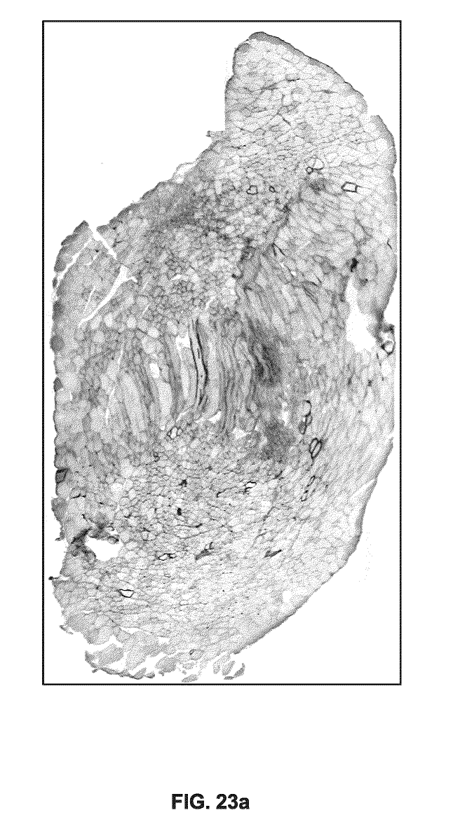

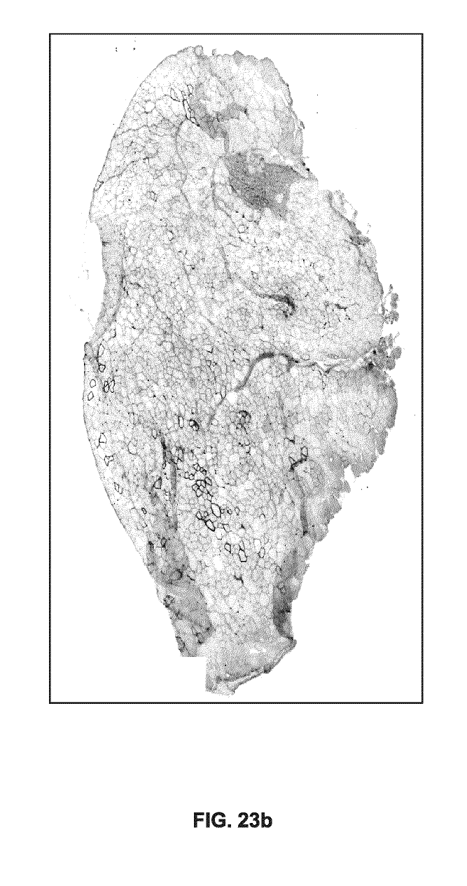

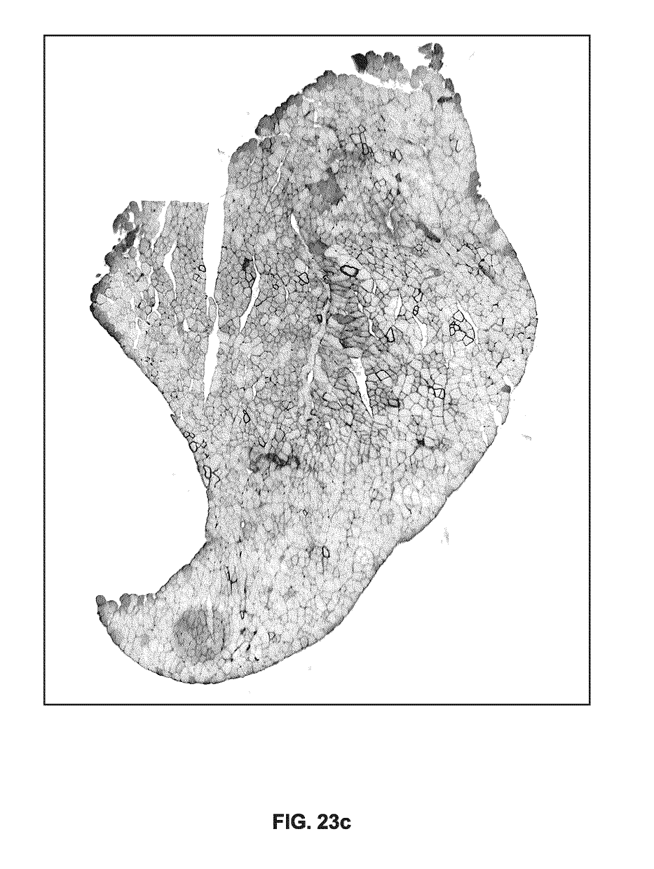

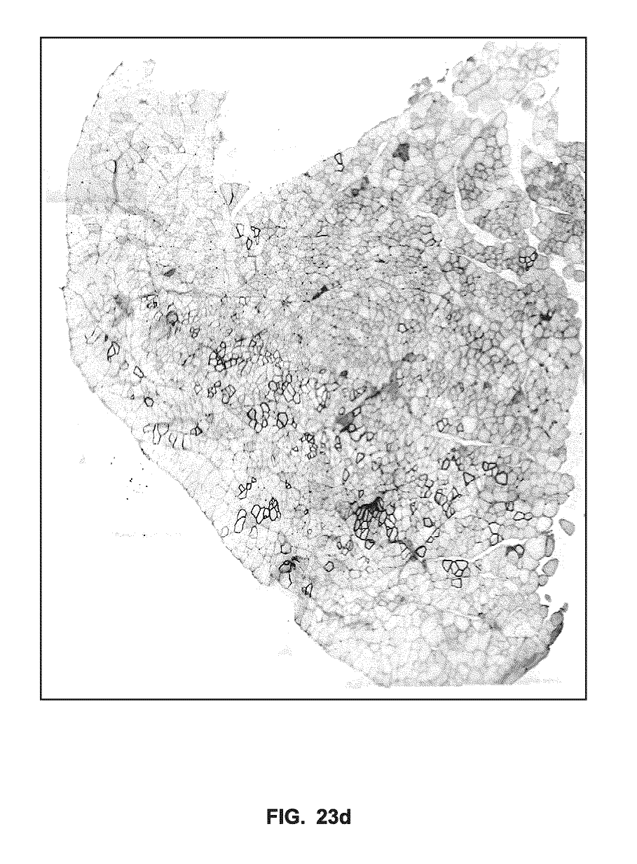

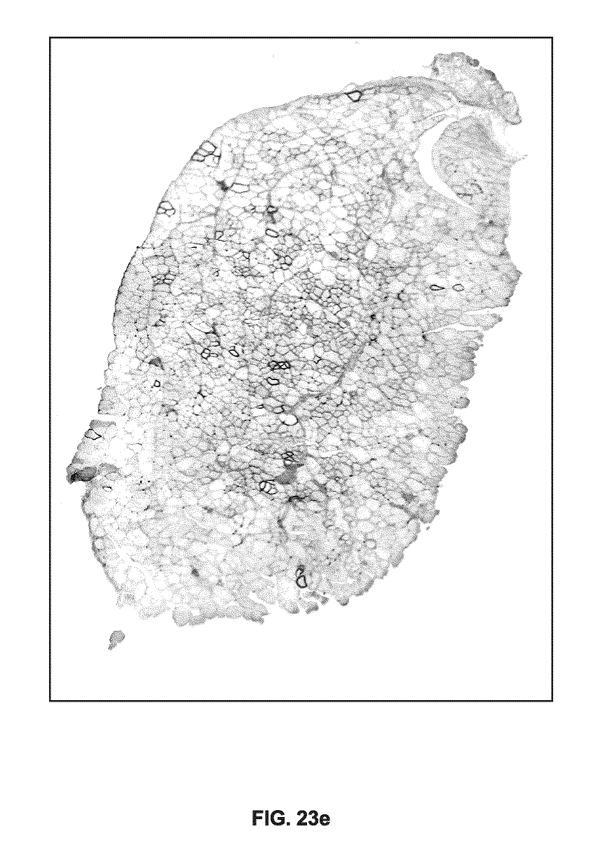

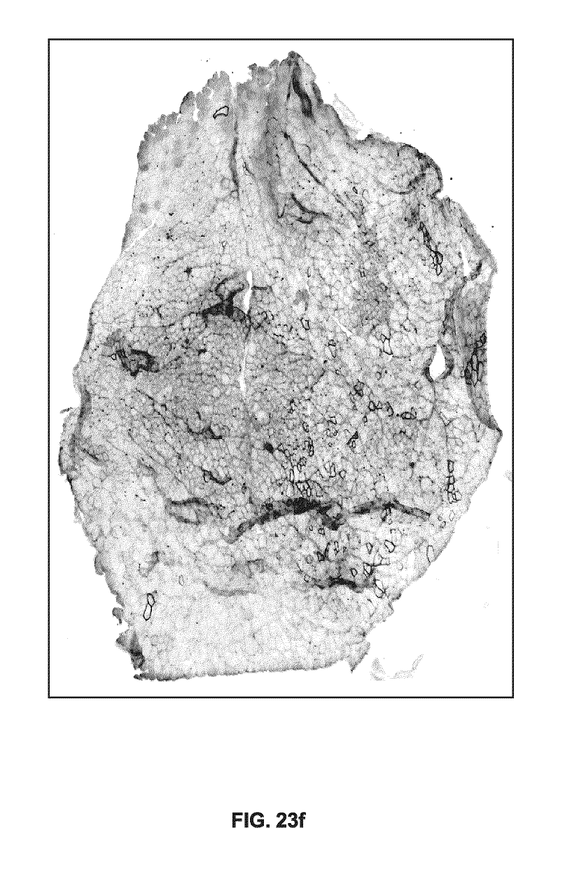

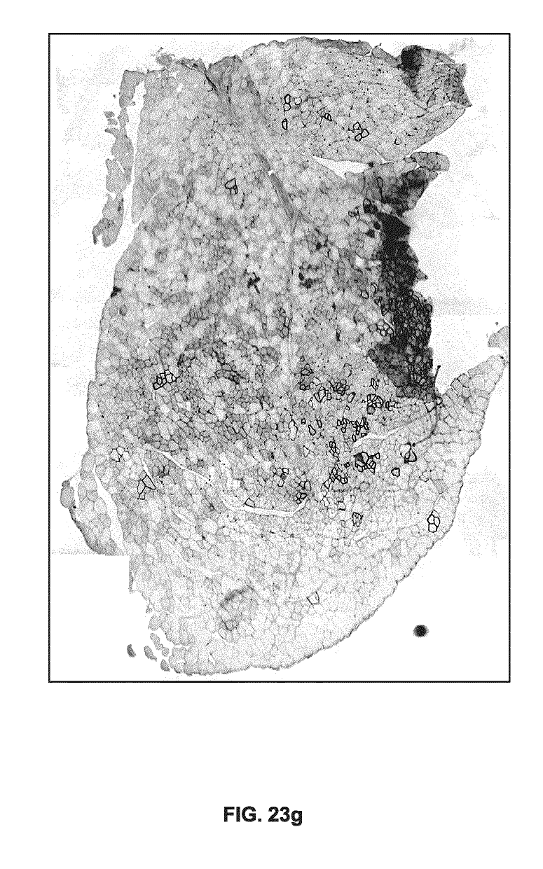

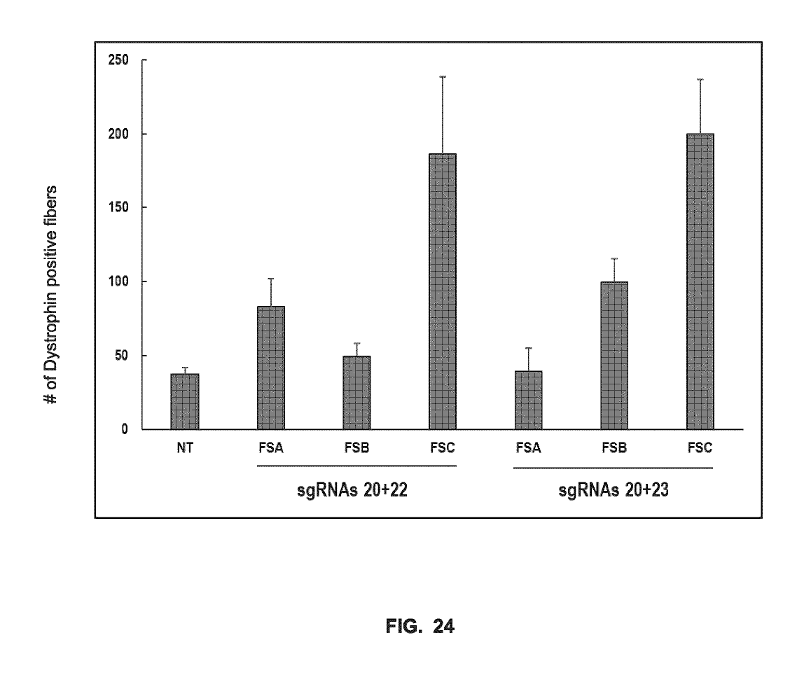

For the first in vivo experiment, 4 month old mice were injected in their Tibialis anterior through a single trans longitudinal injection from the lower to the upper part of the muscle. 3 weeks after the injection, mice were sacrificed for the collection of injected muscles. Even if previous results demonstrated that 3 days post injection we were able to detect the desired genomic deletion, we waited for 3 weeks in order to allow accumulation of the restored dystrophin expression into myotubes. Following muscle collection, harvesting and processing into 16 μm thick transversal cuts, we performed an immunohistochemistry against dystrophin using the NCL-Dys2 antibody (FIG. 23) and analysed dystrophin positive fibers.

We observed that non-treated muscles contain nearly 37 dystrophin positive fibers. These fibers, that naturally occur, are named revertant fibers and might results from another mutation into exon 23 or from a splicing of exon 23, thus correcting the reading frame of the dystrophin gene. In comparison to non-treated muscles, muscles treated with Cas9 and gRNAs 22 and 20 complexed with FSB, or the delivery of Cas9 with gRNAs 23 and 20 complexed with FSA generated an increase in the number of dystrophin positive fibers, respectively showing 49 and 39 dystrophin positive fibers. However, the combination of gRNAs 22 and 20 with FSA and the combination of gRNAs 23 and 20 with the FSB allowed to obtain 83 and 99 dystrophin positive fibers respectively. Finally, the FSC complexed with the ribonucleoprotein delivering the gRNAs 22 and 20 or the gRNAs 23 and 20, exhibited the highest number of dystrophin positive fibers with 186 and 199 fibers, respectively. Consequently, in further experiments we focused on the use of the FSC which seems to be the most suitable non-viral delivery system for the Cas9/gRNAs delivery in the muscle and thus for the correction of the reading frame of the dystrophin gene.

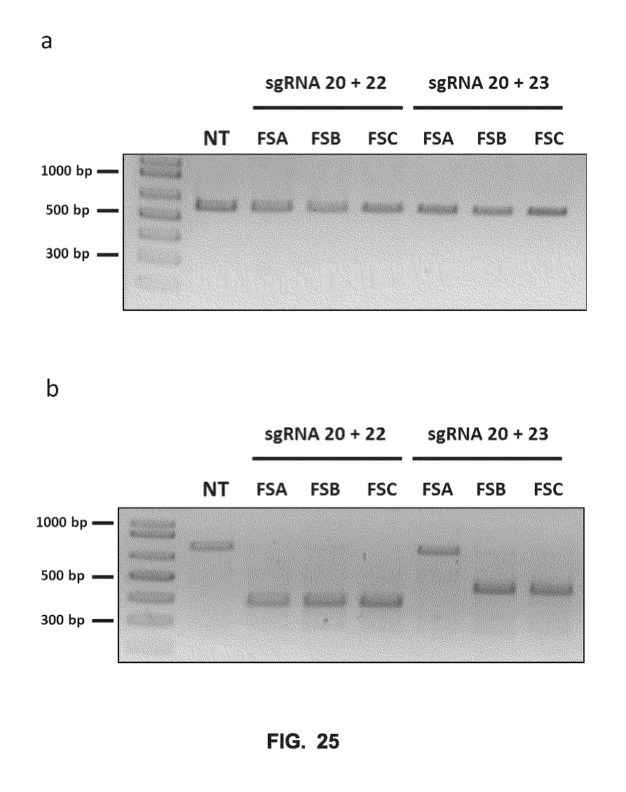

To confirm that the dystrophin positive fibers observed come from the effect of the CRISPR/Cas9 system programmed for the deletion of exon 23, we performed a PCR amplification of the targeted genomic region. As preliminary results of PCR amplification, using the primers Fw2-i22 and Rev1-i23, does not allow the detection of the deletion of the exon 23, we used an optimize nested-PCR approach that allow the detection of a small deletion, as we described in Example 19. Here, the first PCR was made using the primers Fw1-i22 and Rev1-i23 and the poison primer Fw-P (FIG. 25a) only permit the visualization of a product corresponding to a wild type sequence. Then, nested-PCR was performed using internal primers Fw2-i22 and Rev2-i23 (FIG. 25 b). The combination of gRNAs 23 and 20 with the SpCas9 complexed with the FSA does not allow the detection of the deletion of exon 23. However, the other combinations involving the combination of gRNAs with FSB or FSC permitted the detection of a PCR product corresponding to the deletion of exon 23.

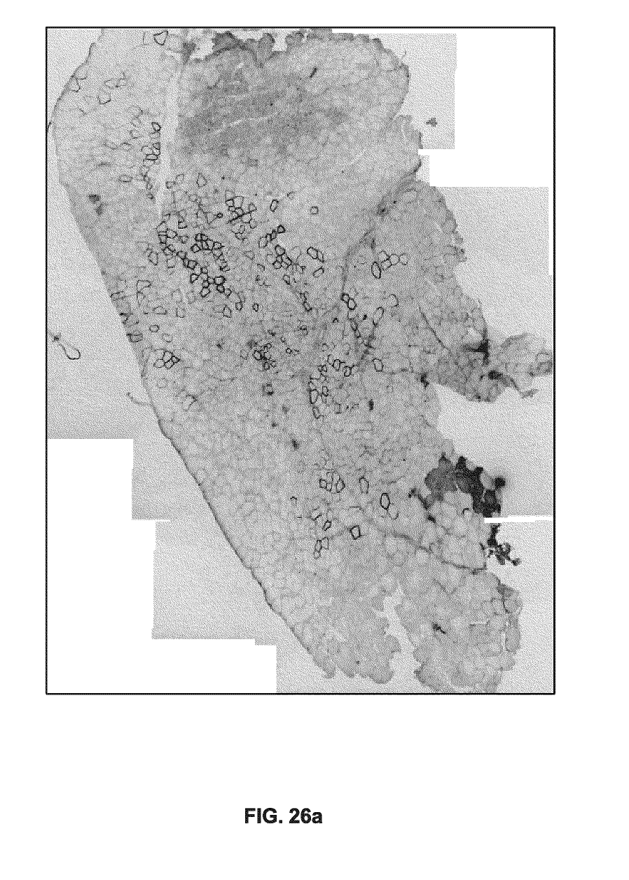

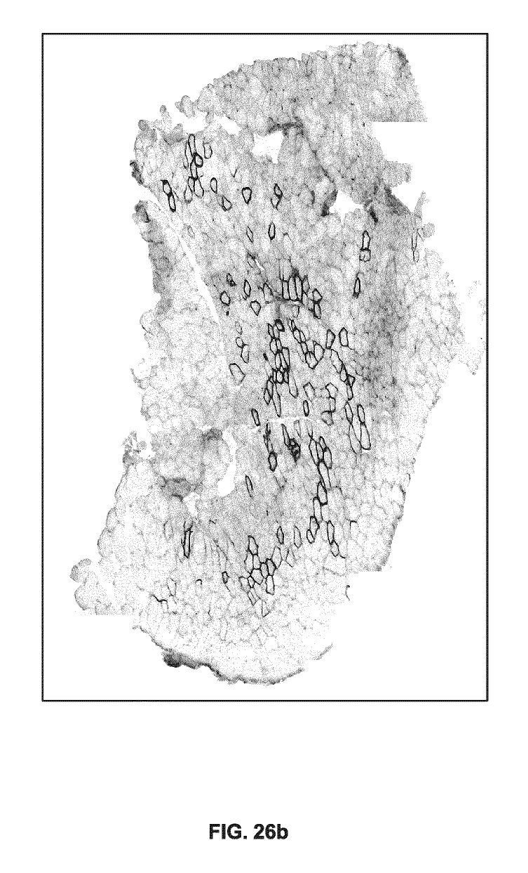

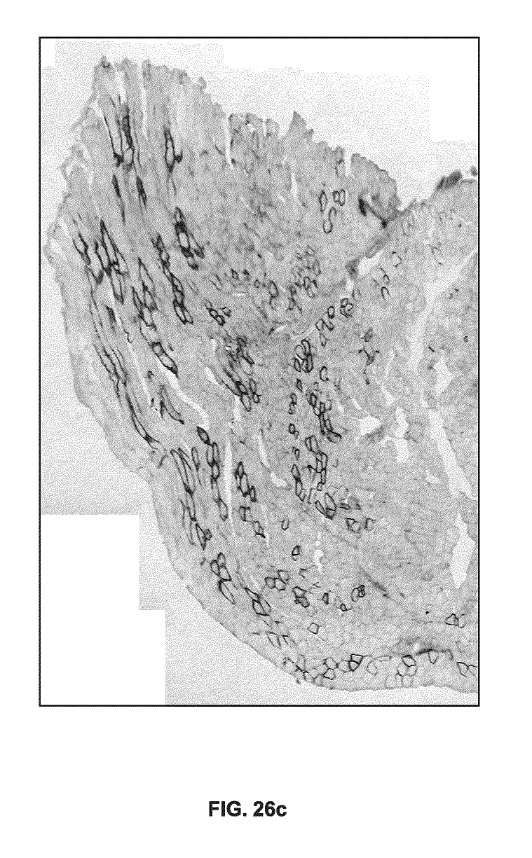

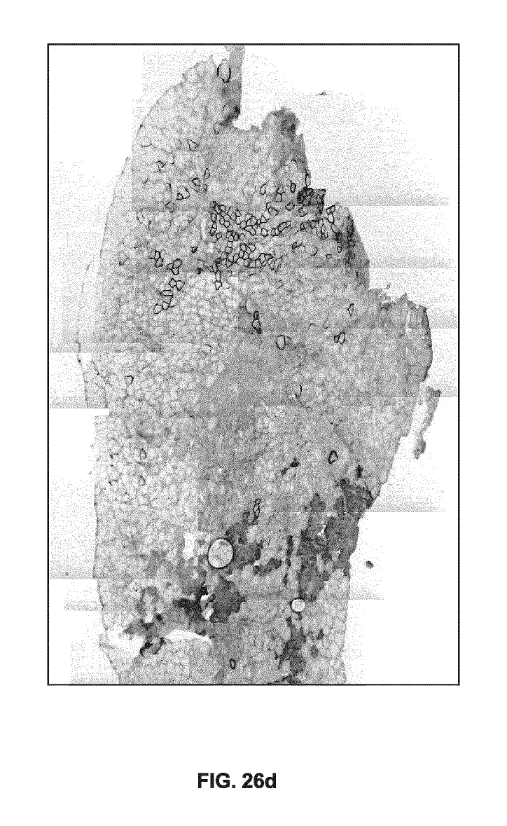

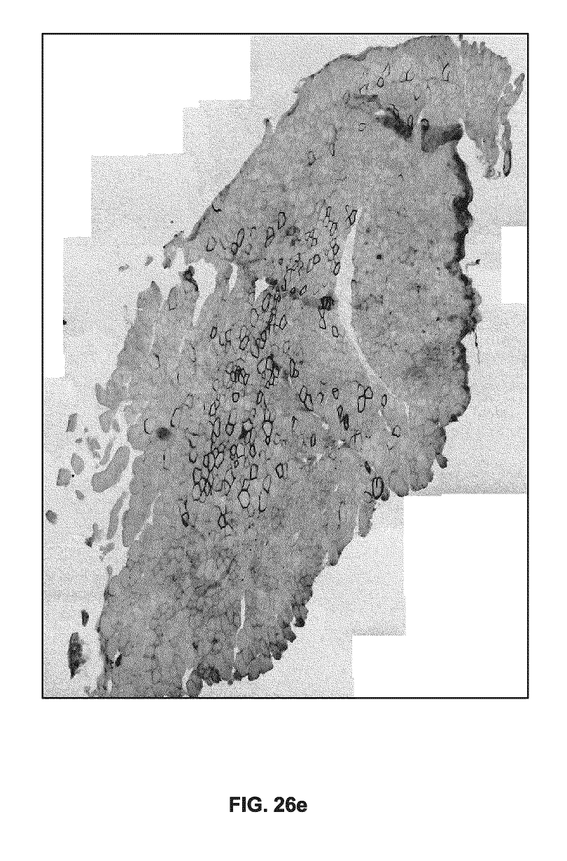

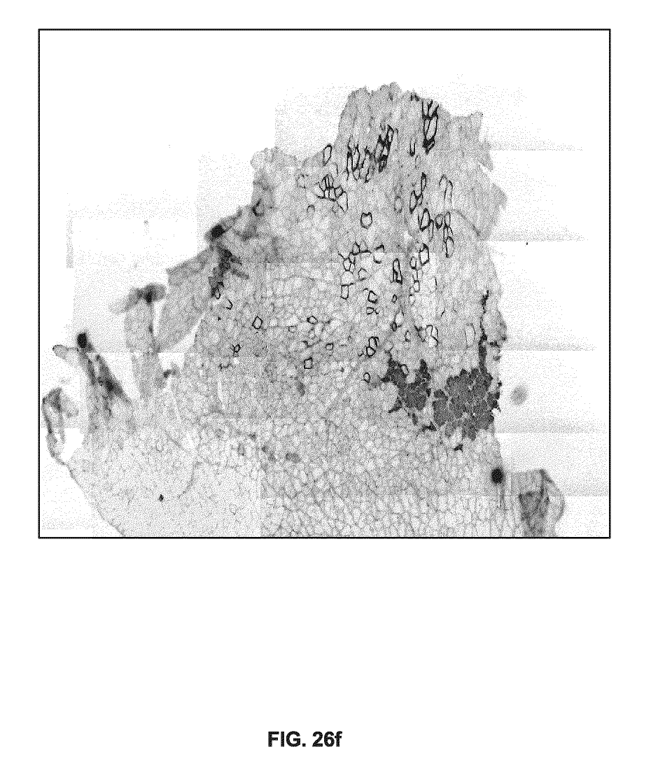

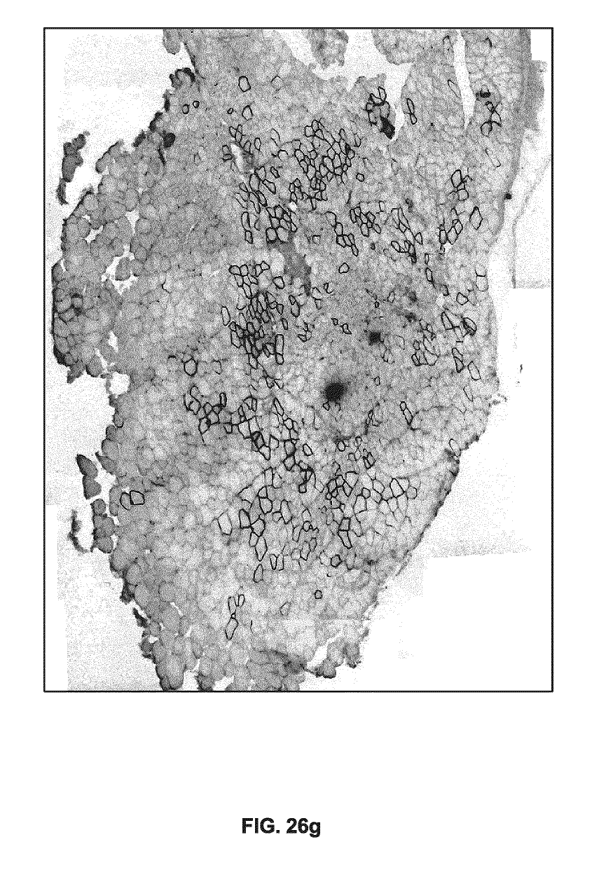

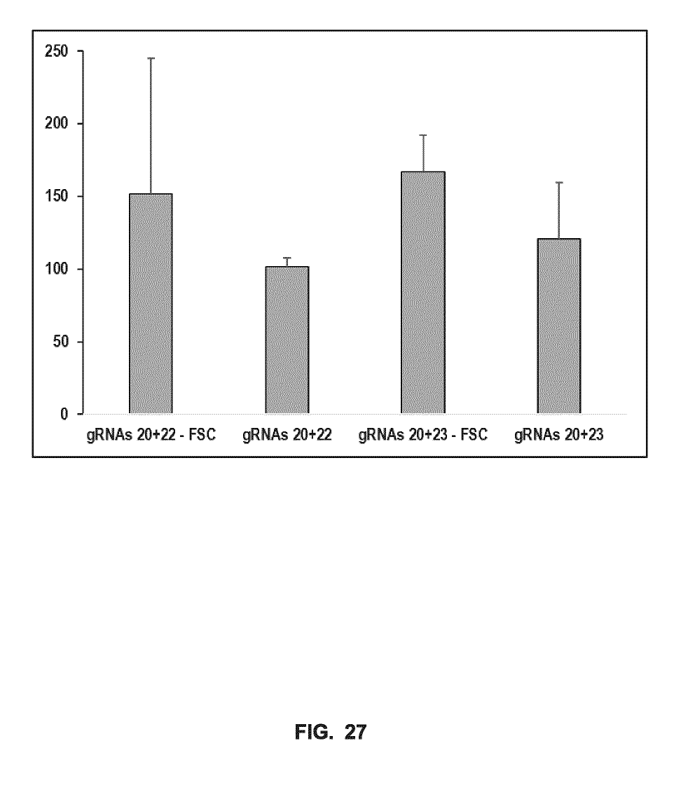

For the second round of in vivo experiments, 6 weeks old mice were injected in their Tibialis anterior through a single trans longitudinal injection. 3 weeks after the injection, mice were sacrificed for the collection of injected muscles. Following muscle collection, harvesting and processing into 16 μm thick transversal cuts, we performed an immunohistochemistry against dystrophin using the NCL-Dys2 antibody (FIG. 26) and analysed dystrophin positive fibers. In these experiment, we injected SpCas9/gRNAs alone or complexed with the Feldan Shuttle FSC that demonstrated the most efficient dystrophin protein recovery in the first experiment.

In these second round experiments, we aimed to compare the delivery of the SpCas9 along with a pair of gRNAs, gRNAs 20 and 22 or gRNAs 20 and 23, complexed or not with FSC. We focused on shuttle FSC as it demonstrated the best delivery efficiency as shown by the elevated number of recovered dystrophin positive fibers detected using this shuttle. Intramuscular injections of SpCas9 complexed with the pair of gRNAs 20 and 22 and the pair of gRNAs 20 and 23 generated 101 and 120 dystrophin positive fibers, respectively. In addition, the use of the FSC for the delivery of the SpCas9 along with a pair of gRNAs permits to increase the number of dystrophin positive fibers. Indeed, the use of FSC for the delivery of the SpCas9 and gRNAs 20 and 22 generated 151 dystrophin positive fibers and the SpCas9 and the gRNAs 20 and 23 generated 166 dystrophin positive fibers.

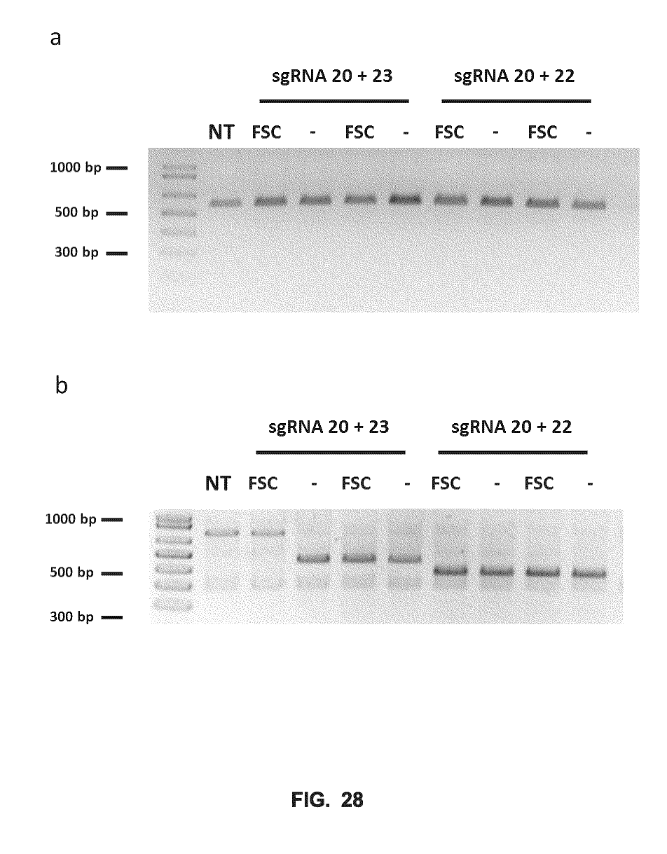

To confirm that the dystrophin positive fibers we observed come from the effect of the CRISPR/Cas9 system and a specific pair of gRNAs for the deletion of exon 23, we performed PCR amplification of the targeted genomic region. The first PCR made using primers Fw1-i22, Rev1-i23 and poison primer Fw-P (FIG. 28a) only allowed amplification of a product corresponding to the wild type DYS sequence. Then, the nested-PCR was performed using internal primers Fw2-i22 and Rev2-i23 (FIG. 28b). The combination of gRNAs 22 and 20 with the SpCas9 alone or complexed with the FSC allowed the detection of the deletion of exon 23. The same observation was made with the combination of gRNAs 23 and 20. However, one injection of the gRNAs 23 and 20 with the SpCas9 complexed with the FSC did not permit the detection of a PCR product corresponding to the deletion of the exon 23 which might result from a failed injection.

TABLE 6
Exemplary gRNAs in exons 46-58. Nucleotides position are provided with reference to the DMD
gene sequence ENS00000198947 (Chromosome X reverse strand 31,097,677-33,339,441) analysed
using Benchling web tool (https://benchling.com/)

| | | | | | | PAM nts | | | |
|---|---|---|---|---|---|---|---|---|---|
| | | | | | | Position | | | |
| | | | | | gRNA | (cs: | | | gRNA involved |
| | Exon | | gRNA target | | target | coding | Cut sites | | in the formation |
| | cutting | | sequences* | SEQ ID NOs. | sequence | sequence, | in DYS | Cut sites in amino | of hybrid |
| gRNA# | site# | Strand | (excluding PAM) | Target/gRNA | position | in: intron) | gene** | acid sequence | exon(s) |
| 1 | 46 | Sense | TTCTCCAGGCTAGAAGAAC | 106/148 | 1407207- | 1407228- | 6624- | 2208 GAA (Glu): | 46-51; 46-53 |
| | | | AA | | 1407227 | 1407233 | 6225 | 2209 CAA (Gln) | |
| 2 | 46 | Anti- | CTGCTCTTTTCCAGGTTCAA | 107/149 | 1407312- | 1407306- | 6714- | 2238 CTT (Leu): | 46-53 |
| | | sense | G | | 1407332 | 1407311 | 6715 | 2239 GAA (Glu) | |
| 3 | 47 | Sense | GTCTGTTTCAGTTACTGGT | 108/150 | 1409686- | 1409707- | 6769- | 2257 G:TG (Val) | 47-58 |
| | | | GG | | 1409706 | 1409712 | 6770 | | |
| 4 | 47 | Anti- | TCCAGTTTCATTTAATTGTT | 109/151 | 1409736- | 1409730- | 6824- | 2268 AAA (Lys): | 47-58 |
| | | sense | T | | 1409756 | 1409735 | 6825 | 2267 CAA (Gln) | |
| 5 | 47 | Anti- | CTTATGGGAGCACTTACAA | 110/152 | 1409765- | 1409759- | 6833- | 2278 CT:T (Leu) | 47-58 |
| | | sense | GC | | 1409785 | 1409764 | 6834 | | |
| 6 | 49 | Anti- | TTGCTTCATTACCTTCACTG | 111/153 | 1502716- | 1502710- | 7194- | 2398 CCA (Pro): | 49-52; 49-53 |
| | | sense | G | | 1502736 | 1502715 | 7195 | 2399 GTG (Val) | |
| 7 | 51 | Anti- | TTGTGTCACCAGAGTAACA | 112/154 | 1565282- | 1565276- | 7323- | 2441 ACT (Thr): | 46-51 |
| | | sense | GT | | 1565302 | 1564281 | 7324 | 2442 GTT (Val) | |
| 8 | 51 | Anti- | AGTAACCACAGGTTGTGTC | 113/155 | 1565294- | 1565288- | 7335- | 2445 GTG (Val): | 46-51 |
| | | sense | AC | | 1565314 | 1565293 | 7336 | 2446 ACA (Thr) | |
| 9 | 52 | Anti- | TTCAAATTTTGGGCAGCGG | 114/156 | 1609765- | 1609759- | 7595- | 2532 AC:C (Thr) | 47-52 |
| | | sense | TA | | 1609785 | 1609764 | 7596 | | |
| 10 | 52 | Sense | CAAGAGGCTAGAACAATCA | 115/157 | 1609802- | 1609823- | 7647- | 2549 ATC (Ile): | 49-52 |
| | | | TT | | 1609822 | 1609828 | 7648 | 2550 ATT (Ile) | |
| 11 | 53 | Anti- | TTGTACTTCATCCCACTGAT | 116/158 | 1659891- | 1659885- | 7677- | 2559 AAT (Asn): | 49-53 |
| | | sense | T | | 1659911 | 1659890 | 7678 | 2560 CAG (Gln) | |
| 12 | 53 | Sense | CTTCAGAACCGGAGGCAAC | 117/159 | 1659918- | 1659939- | 7719- | 2573 CAA (Gln): | 46-53 |
| | | | AG | | 1659938 | 1659944 | 7720 | 2574 CAG (Gln) | |
| 13 | 53 | Sense | CAACAGTTGAATGAAATGTT | 118/160 | 1659933- | 1659954- | 7734- | 2578 ATG (Met): | 46-53 |
| | | | A | | 1659953 | 1659959 | 7735 | 2579 TTA (Leu) | |
| 14 | 53 | Sense | GCCAAGCTTGAGTCATGGA | 119/161 | 1660017- | 1660038- | 7818- | 2606 TGG (Trp): | 46-53 |
| | | | AG | | 1660037 | 1660043 | 7819 | 2607 AAG (Lys) | |
| 15 | 53 | Anti- | CTTGGTTTCTGTGATTTTCT | 120/162 | 1660068- | 1660062- | 7854- | 2618 AAG (Lys): | 46-53 |
| | | sense | T | | 1660088 | 1660067 | 7855 | 2619 AAA (Lys) | |
| 16 | 58 | Sense | TCATTTCACAGGCCTTCAA | 121/163 | 1860349- | 1860370- | 8554- | 2852 A:AG (Lys) | 47-58 |
| | | | GA | | 1860369 | 1860375 | 8555 | | |
| 17 | 58 | Anti- | CAGAAATATTCGTACAGTCT | 122/164 | 1860411- | 1860405- | 8601- | 2867 GAG (Gln): | 47-58 |
| | | sense | C | | 1860431 | 1860410 | 8602 | 2868 ACT (Thr) | |
| 18 | 58 | Anti- | CAATTACCTCTGGGCTCCT | 123/165 | 1860467- | 1860461- | 8657- | 2886 CA:G (Gln) | 47-58 |
| | | sense | GG | | 1860487 | 1860466 | 8658 | | |

*sequences shown in bold are intronic sequences (i.e., portions adjacent to the indicated exon)
**Numbering with reference to the DYS cDNA sequence found at http://www.dmd.nl/seqs/murefDMD.html (Leiden Muscular Dystrophy pages, DMD cDNA Reference sequence). The reference sequence is based on GenBank file NM_004006.1 (with one difference 12505G > A), containing the Dp427m isoform (muscle) of dystrophin.

TABLE 7
Hybrid exon junctions following introduction of first and second cuts with various gRNA pairs

| | | | Part of the | | | New |
|---|---|---|---|---|---|---|
| | Hybrid | Part of the | downstream | | New | amino |
| Combinations | exons | upstream exon | exon | Observation | codon | acid |
| gRNA 1/gRNA 7 | 46-51 | Glu | Val | Junction Glu - Val | None | None |
| gRNA 6/gRNA 8 | 46-51 | Pro | Thr | Junction Pro - Thr | None | None |
| gRNA 1/gRNA 12 | 46-53 | Glu | Gln | Junction Glu - Gln | None | None |
| gRNA 1/gRNA 13 | 46-53 | Glu | Gln | Junction Glu - Gln | None | None |
| gRNA 2/gRNA 14 | 46-53 | Leu | Lys | Junction Leu - Lys | None | None |
| gRNA 2/gRNA 15 | 46-53 | Leu | Lys | Junction Leu - Lys | None | None |
| gRNA 5/gRNA 9 | 47-52 | CT | C | CT + C = CTC | CTC | Leu |
| gRNA 6/gRNA 10 | 49-52 | Pro | Glu | Junction Pro - Glu | None | None |
| gRNA 6/gRNA 11 | 49-53 | Pro | Glu | Junction Pro - Glu | None | None |
| gRNA 3/gRNA 16 | 47-58 | G | AG | G + AG = GAG | GAG | Glu |
| gRNA 4/gRNA 17 | 47-58 | Gln | Thr | Junction Gln - Thr | None | None |
| gRNA 5/gRNA 18 | 47-58 | CT | G | CT + G = CTG | CTG | Leu |

TABLE 8
Exemplary gRNAs targeting introns 22 and 23. Nucleotides position are provided with
reference to the mouse DMD gene sequence ENSMUSG00000045103 (analysed using Benchling web
tool (https://benchling.com/)

| | intron | | | | gRNA | | |
|---|---|---|---|---|---|---|---|
| | cutting | | gRNA target sequences | SEQ ID NOs. | target | PAM nt | Cut site |
| #gRNA | site | Strand | (excluding PAM) | Target/gRNA | position | position | localization |
| 19 | 22 | Sense | GAACTTCTATTTAATTTTG | 206/214 | 83803285- | 83803304- | 83803300- |
| | | | | | 83803303 | 83803306 | 83803301 |
| 20 | 23 | Sense | ATTTCAGGTAAGCCGAGGTT | 207/215 | 83803512- | 83803532- | 83803528- |
| | | | | | 83803531 | 83803534 | 83803529 |
| 21 | 22 | Sense | TCTTAATAATGTTTCACTGT | 208/216 | 83803014- | 83803034- | 83803030- |
| | | | | | 83803033 | 83803036 | 83803031 |
| 22 | 22 | Anti- | ATAATTTCTATTATATTACA | 209/217 | 83803137- | 83803134- | 83803139- |
| | | sense | | | 83803156 | 83803136 | 83803140 |
| 23 | 22 | Anti- | TTTCATTCATATCAAGAAGA | 210/218 | 83803237- | 83803234- | 83803239- |
| | | sense | | | 83803256 | 83803236 | 83803240 |
| 24 | 23 | Anti- | ATAGTTTAAAGGCCAAACCT | 211/219 | 83803527- | 83803524- | 83803529- |
| | | sense | | | 83803546 | 83803526 | 83803530 |
| 25 | 23 | Anti- | TGTGAAAAAATATAGTTTAA | 212/220 | 83803538- | 83803535- | 83803540- |
| | | sense | | | 83803557 | 83803537 | 83803541 |
| 26 | 23 | Sense | CGAAAATTTCAGGTAAGCCG | 213/221 | 83803507- | 83803527- | 83803523- |
| | | | | | 83803526 | 83803529 | 83803524 |

*sequences shown in bold are exonic sequences (i.e., portions adjacent to the indicated intron)

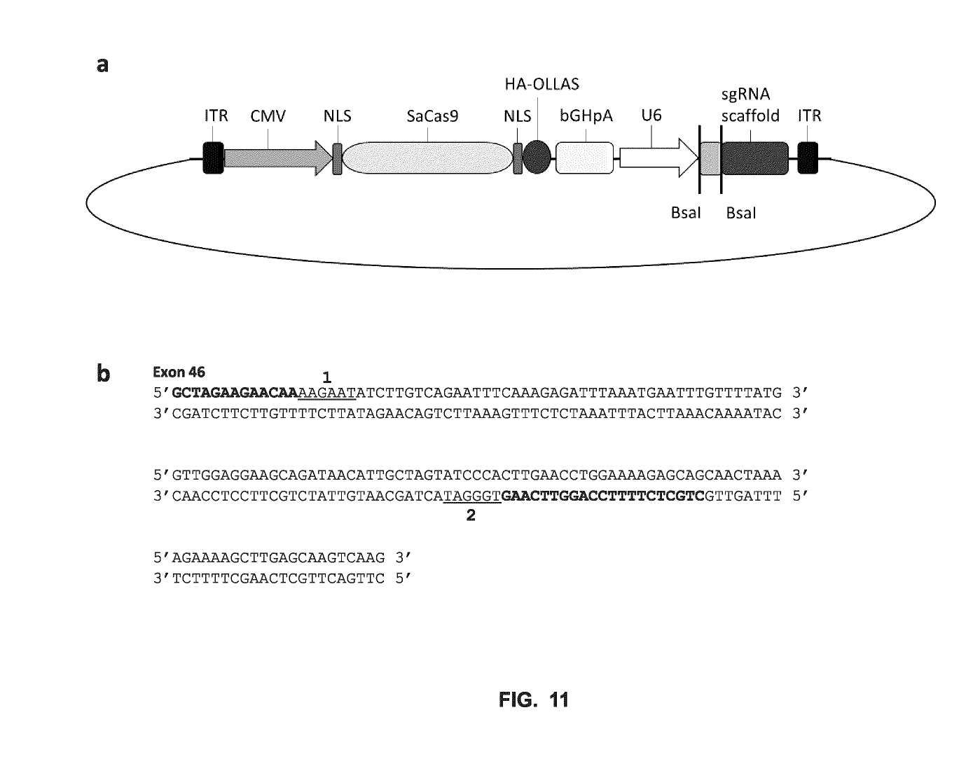

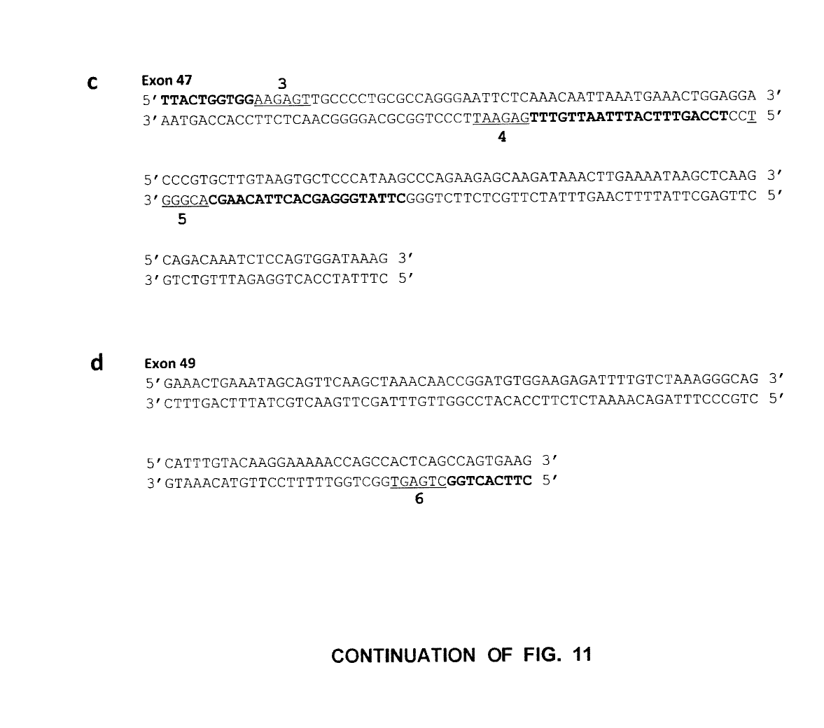

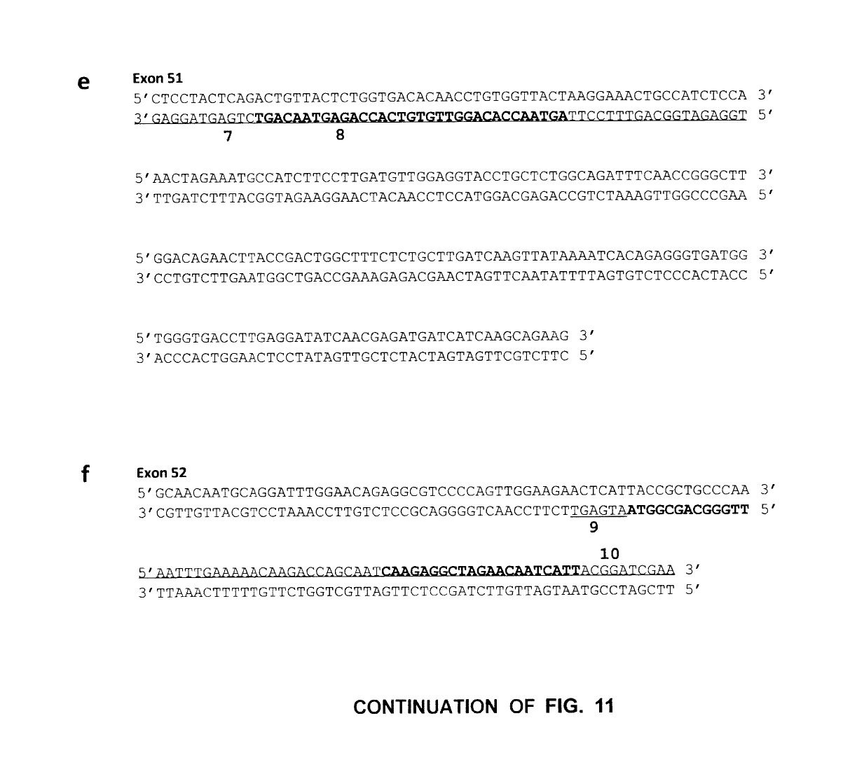

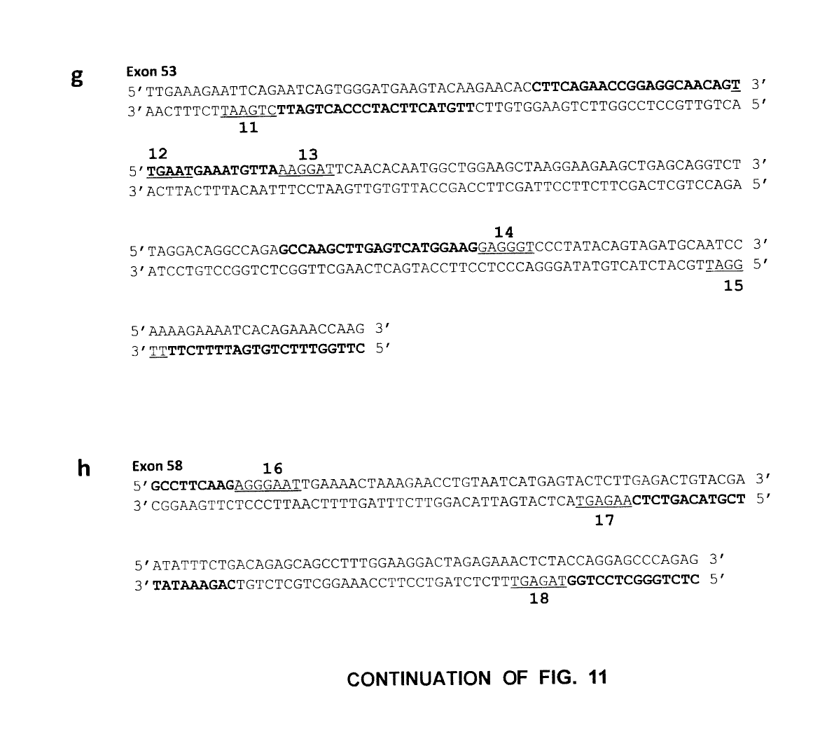

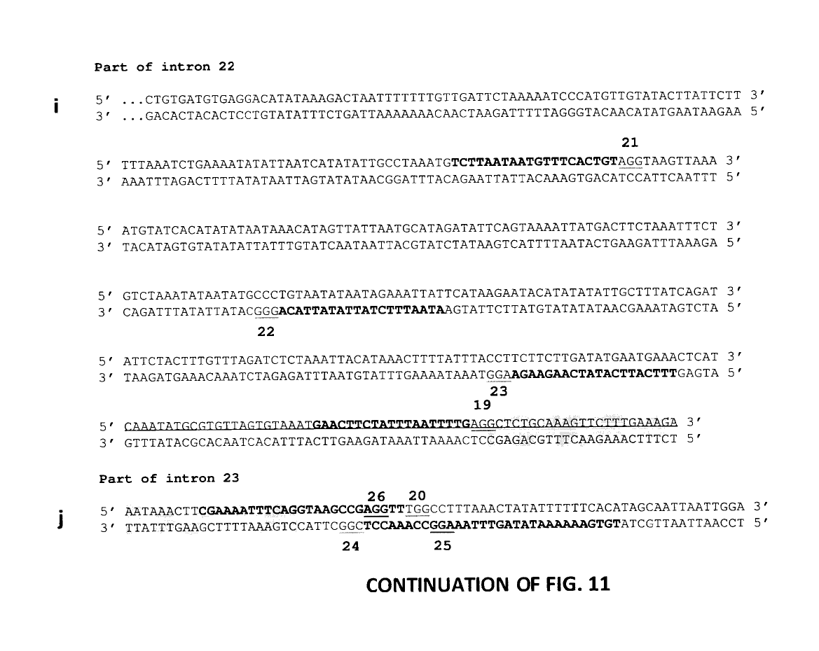

TABLE 9
Sequences described herein

| SEQ ID | |
|---|---|
| NO(s) | Description |
| 1 | Dystrophin DMD-001 cDNA Ensembl (ENSG00000198947) (from Start (ATG) to Stop (TAG) codon |
| 2 | Dystrophin protein sequence DMD-001 (Translation of SEQ ID NO: 1) |
| 3 | 25 nts of 5′ UTR + cDNA sequence of exon 1 + 25 nts of adjacent 3′ intron sequence of Dystrophin |
| | transcript (DMD-001) |
| 4-80 | cDNA exon sequences (exons 2 to 78) of Dystrophin transcript (DMD-001) with flanking 25 nts of intron |
| | sequences on each side (5′ and 3′) of each exon |
| 81 | cDNA of exon 79 sequence flanked by 25 nts of adjacent intron sequence in 5′ and 25 nts of 3′UTR |
| | sequence in 3′ |
| 82-105 | gRNA target sequences on the Dystrophin gene listed in Table 3 (Example 2) |
| 106-123 | gRNA target sequences on the Dystrophin gene listed in Table 6. SEQ ID NO: 107 (target sequence of |
| | “gRNA3”); SEQ ID NO: 109 (target sequence of “gRNA5”); SEQ ID NO: 120 (Target sequence of |
| | “gRNA16”); and SEQ ID NO: 122 (target sequence of “gRNA18”) |
| 124-147 | gRNA RNA sequences corresponding to the target sequences of SEQ ID NOs: 82-104 listed in Table 3 |
| | (Example 2) |
| 148-165 | gRNA RNA sequences of the target sequences of SEQ ID NOs: 105-122 listed in Table 6 (Example |
| | 19). SEQ ID NO: 149 (“gRNA3”); SEQ ID NO: 151 (“gRNA5”); SEQ ID NO: 162 (“gRNA16”); and SEQ |
| | ID NO: 164 (“gRNA18”) |
| 166 | S. pyogenes Cas9 RNA recognition sequence (TracrRNA/crRNA) |
| 167 | Sequence of plasmid pX601-AAV-CMV::NLS-SaCas9-NLS-3xHA-bGHpA; U6::Bsal-sgRNA |
| | (Addgene Plasmid # 61591) |
| 168 | Cpf1 recognition sequence (TracrRNA) |
| 169 | Protein sequence of humanized Cas 9 from S. pyogenes (without NLS and without TAG) |
| 170 | Protein sequence of humanized Cas9 from S. pyogenes (with NLS and without TAG) |
| 171 | Protein sequence of humanized Cas 9 from S. aureus (without NLS and without TAG) |
| 172 | Protein sequence of humanized Cas 9 from S. aureus (with NLS and without TAG) |
| 173-177 | Primer sequences listed in Example 1 |
| 178-195 | Primer sequences listed in Example 8 |
| 196-198 | Feldan Shuttles A, B and C, respectively |
| 199-200 | Expected and obtained sequence of hybrid exon 50-54 (FIG. 9) |
| 201-203 | Primers for amplification of in vitro transcription templates (Table 5, Example 8) |
| 204-205 | Primers used for PCR amplification Fw2-i22 (SEQ ID NO: 204); Rev1-i23 (SEQ ID NO: 205) |
| 206-213 | Target sequence of gRNAs listed in Table 8 |
| 214-221 | RNA sequences of gRNA listed in Table 8 |
| 222 | Primer used for HDMD Hybrid Exon Analysis by PCR (Fw-Int50)) |
| 223-227 | Primers for PCR amplification of the hybrid introns (see Example 19) |
| 228 | Partial intron 22 and exon 23 sequence shown in FIG. 11i |
| 229 | Partial exon 23 and intron 23 sequence shown in FIG. 11j |

Although the present invention has been described hereinabove by way of preferred embodiments thereof, it can be modified, without departing from the spirit and nature of the subject invention as defined in the appended claims.

### REFERENCES

1. Engel, A, and Banker, B Q (1986). Myology: basic and clinical, McGraw-Hill: New York.
2. Rybakova, I N, Patel, J R, and Ervasti, J M (2000). The dystrophin complex forms a mechanically strong link between the sarcolemma and costameric actin. J Cell Biol 150: 1209-1214.
3. Hoffman, E P, Brown, R H, Jr., and Kunkel, L M (1987). Dystrophin: the protein product of the Duchenne muscular dystrophy locus. Cell 51: 919-928.
4. Hoffman, E P, Brown, R H, and Kunkel, L M (1992). Dystrophin: the protein product of the Duchene muscular dystrophy locus. 1987 [classical article]. Biotechnology 24: 457-466.
5. Bladen, C L, Salgado, D, Monges, S, Foncuberta, M E, Kekou, K, Kosma, K, et al. (2015). The TREAT-NMD DMD Global Database: analysis of more than 7,000 Duchenne muscular dystrophy mutations. Hum Mutat 36: 395-402.
6. Koenig, M, Beggs, A H, Moyer, M, Scherpf, S, Heindrich, K, Bettecken, T, et al. (1989). The molecular basis for Duchenne versus Becker muscular dystrophy: correlation of severity with type of deletion. Am J Hum Genet 45: 498-506.
7. Hoffman, E P (1993). Genotype/phenotype correlations in Duchenne/Becker dystrophy. Mol Cell Biol Hum Dis Ser 3: 12-36.
8. Emery, A E (2002). The muscular dystrophies. Lancet 359: 687-695.
9. Duan, D (2011). Duchenne muscular dystrophy gene therapy: Lost in translation? Research and reports in biology 2011: 31-42.
10. Goyenvalle, A, Seto, J T, Davies, K E, and Chamberlain, J (2011). Therapeutic approaches to muscular dystrophy. Hum Mol Genet 20: R69-78.
11. Konieczny, P, Swiderski, K, and Chamberlain, J S (2013). Gene and cell-mediated therapies for muscular dystrophy. Muscle Nerve 47: 649-663.
12. Mendell, J R, et al. (2012). Gene therapy for muscular dystrophy: Lessons learned and path forward. Neurosci Lett 527: 90-99.
13. Verhaart, I E, and Aartsma-Rus, A (2012). Gene therapy for Duchenne muscular dystrophy. Curr Opin Neurol 25: 588-596.
14. Monaco, A P, Neve, R L, Colletti-Feener, C, Bertelson, C J, Kurnit, D M, and Kunkel, L M (1986). Isolation of candidate cDNAs for portions of the Duchenne muscular dystrophy gene. Nature 323: 646-650.
15. Kunkel, L M, Hejtmancik, J F, Caskey, C T, Speer, A, Monaco, A P, Middlesworth, W, et al. (1986). Analysis of deletions in DNA from patients with Becker and Duchenne muscular dystrophy. Nature 322: 73-77.
16. Gregorevic, P, Blankinship, M J, Allen, J M, Crawford, R W, Meuse, L, Miller, D G, et al. (2004). Systemic delivery of genes to striated muscles using adeno-associated viral vectors. Nat Med 10: 828-834.
17. Gregorevic, P, et al. (2006). rAAV6-microdystrophin preserves muscle function and extends lifespan in severely dystrophic mice. Nat Med 12: 787-789.
18. Wang, Z, Kuhr, C S, Allen, J M, Blankinship, M, Gregorevic, P, Chamberlain, J S, et al. (2007). Sustained AAV-mediated dystrophin expression in a canine model of Duchenne muscular dystrophy with a brief course of immunosuppression. Mol Ther 15: 1160-1166.
19. Qiao, C, Koo, T, Li, J, Xiao, X, and Dickson, J G (2011). Gene therapy in skeletal muscle mediated by adeno-associated virus vectors. Methods Mol Biol 807: 119-140.
20. Aartsma-Rus, A (2012). Overview on DMD exon skipping. Methods Mol Biol 867: 97-116.
21. Aartsma-Rus, A, and van Ommen, G J (2007). Antisense-mediated exon skipping: a versatile tool with therapeutic and research applications. RNA 13: 1609-1624.
22. Aartsma-Rus, A, and van Ommen, G J (2009). Less is more: therapeutic exon skipping for Duchenne muscular dystrophy. Lancet Neurol 8: 873-875.
23. Dunckley, M G, et al. (1998). Modification of splicing in the dystrophin gene in cultured Mdx muscle cells by antisense oligoribonucleotides. Hum Mol Genet 7: 1083-1090.
24. Lu, Q L, Mann, C J, Lou, F, Bou-Gharios, G, Morris, G E, Xue, S A, et al. (2003). Functional amounts of dystrophin produced by skipping the mutated exon in the mdx dystrophic mouse. Nat Med 9: 1009-1014.
25. Mann, C J, Honeyman, K, McClorey, G, Fletcher, S, and Wilton, S D (2002). Improved antisense oligonucleotide induced exon skipping in the mdx mouse model of muscular dystrophy. J Gene Med 4: 644-654.
26. Takeshima, Y, Yagi, M, Wada, H, Ishibashi, K, Nishiyama, A, Kakumoto, M, et al. (2006). Intravenous infusion of an antisense oligonucleotide results in exon skipping in muscle dystrophin mRNA of Duchenne muscular dystrophy. Pediatr Res 59: 690-694.
27. van Deutekom, J C, Janson, A A, Ginjaar, I B, Frankhuizen, W S, Aartsma-Rus, A, Bremmer-Bout, M, et al. (2007). Local dystrophin restoration with antisense oligonucleotide PRO051. The New England journal of medicine 357: 2677-2686.
28. Kinali, M, Arechavala-Gomeza, V, Feng, L, Cirak, S, Hunt, D, Adkin, C, et al. (2009). Local restoration of dystrophin expression with the morpholino oligomer AVI-4658 in Duchenne muscular dystrophy: a single-blind, placebo-controlled, dose-escalation, proof-of-concept study. Lancet Neurol 8: 918-928.
29. Aartsma-Rus, A (2010). Antisense-mediated modulation of splicing: therapeutic implications for Duchenne muscular dystrophy. RNA biology 7: 453-461.
30. Ousterout, D G, Kabadi, A M, Thakore, P I, Perez-Pinera, P, Brown, M T, Majoros, W H, et al. (2015). Correction of dystrophin expression in cells from duchenne muscular dystrophy patients through genomic excision of exon 51 by zinc finger nucleases. Mol Ther 23: 523-532.
31. Rousseau, J, Chapdelaine, P, Boisvert, S, Almeida, L P, Corbeil, J, Montpetit, A, et al. (2011). Endonucleases: tools to correct the dystrophin gene. J Gene Med 13: 522-537.
32. Li, H L, Fujimoto, N, Sasakawa, N, Shirai, S, Ohkame, T, Sakuma, T, et al. (2015). Precise correction of the dystrophin gene in duchenne muscular dystrophy patient induced pluripotent stem cells by TALEN and CRISPR-Cas9. Stem cell reports 4: 143-154.
33. Ousterout, D G, Perez-Pinera, P, Thakore, P I, Kabadi, A M, Brown, M T, Qin, X, et al. (2013). Reading frame correction by targeted genome editing restores dystrophin expression in cells from Duchenne muscular dystrophy patients. Mol Ther 21: 1718-1726.
34. Long, C, McAnally, J R, Shelton, J M, Mireault, A A, Bassel-Duby, R, and Olson, E N (2014). Prevention of muscular dystrophy in mice by CRISPR/Cas9-mediated editing of germline DNA. Science 345: 1184-1188.
35. Nakamura, K, Fujii, W, Tsuboi, M, Tanihata, J, Teramoto, N, Takeuchi, S, et al. (2014). Generation of muscular dystrophy model rats with a CRISPR/Cas system. Scientific reports 4: 5635.
36. Ousterout, D G, Kabadi, A M, Thakore, P I, Majoros, W H, Reddy, T E, and Gersbach, C A (2015). Multiplex CRISPR/Cas9-based genome editing for correction of dystrophin mutations that cause Duchenne muscular dystrophy. Nature communications 6: 6244.
37. Ran, F A, Cong, L, Yan, W X, Scott, D A, Gootenberg, J S, Kriz, A J, et al. (2015). In vivo genome editing using Staphylococcus aureus Cas9. Nature 520: 186-191.
38. Cong, L, Ran, F A, Cox, D, Lin, S, Barretto, R, Habib, N, et al. (2013). Multiplex genome engineering using CRISPR/Cas systems. Science 339: 819-823.
39. Jinek, M, East, A, Cheng, A, Lin, S, Ma, E, and Doudna, J (2013). RNA-programmed genome editing in human cells. eLife 2: e00471.
40. Sander, J D, and Joung, J K (2014). CRISPR-Cas systems for editing, regulating and targeting genomes. Nat Biotechnol 32: 347-355.
41. Mali, P, Yang, L, Esvelt, K M, Aach, J, Guell, M, DiCarlo, J E, et al. (2013). RNA-guided human genome engineering via Cas9. Science 339: 823-826.
42. Cho, S W, Kim, S, Kim, J M, and Kim, J S (2013). Targeted genome engineering in human cells with the Cas9 RNA-guided endonuclease. Nat Biotechnol.
43. Doudna, J A, and Charpentier, E (2014). Genome editing. The new frontier of genome engineering with CRISPR-Cas9. Science 346: 1258096.
44. Zheng, Q, Cai, X, Tan, M H, Schaffert, S, Arnold, C P, Gong, X, et al. (2014). Precise gene deletion and replacement using the CRISPR/Cas9 system in human cells. Biotechniques 57: 115-124.
45. Deltcheva, E, Chylinski, K, Sharma, C M, Gonzales, K, Chao, Y, Pirzada, Z A, et al. (2011). CRISPR RNA maturation by trans-encoded small RNA and host factor RNase Ill. Nature 471: 602-607.
46. Marraffini, L A, and Sontheimer, E J (2010). CRISPR interference: RNA-directed adaptive immunity in bacteria and archaea. Nat Rev Genet 11: 181-190.
47. Jinek, M, Chylinski, K, Fonfara, I, Hauer, M, Doudna, J A, and Charpentier, E (2012). A programmable dual-RNA-guided DNA endonuclease in adaptive bacterial immunity. Science 337: 816-821.
48. Canver, M C, Bauer, D E, Dass, A, Yien, Y Y, Chung, J, Masuda, T, et al. (2014). Characterization of genomic deletion efficiency mediated by clustered regularly interspaced palindromic repeats (CRISPR)/Cas9 nuclease system in mammalian cells. J Biol Chem 289: 21312-21324.
49. Aartsma-Rus, A, Kaman, W E, Weij, R, den Dunnen, J T, van Ommen, G J, and van Deutekom, J C (2006). Exploring the frontiers of therapeutic exon skipping for Duchenne muscular dystrophy by double targeting within one or multiple exons. Mol Ther 14: 401-407.
50. Beroud, C, Tuffery-Giraud, S, Matsuo, M, Hamroun, D, Humbertclaude, V, Monnier, N, et al. (2007). Multiexon skipping leading to an artificial DMD protein lacking amino acids from exons 45 through 55 could rescue up to 63% of patients with Duchenne muscular dystrophy. Hum Mutat 28: 196-202.
51. Skuk, D, and Tremblay, J P (2014). Clarifying misconceptions about myoblast transplantation in myology. Mol Ther 22: 897-898.
52. Skuk, D, and Tremblay, J P (2011). Intramuscular cell transplantation as a potential treatment of myopathies: clinical and preclinical relevant data. Expert Opin Biol Ther 11: 359-374.
53. Bruusgaard, J C, Liestol, K, Ekmark, M, Kollstad, K, and Gundersen, K (2003). Number and spatial distribution of nuclei in the muscle fibres of normal mice studied in vivo. J Physiol 551: 467-478.
54. Kinoshita, I, Vilquin, J T, Asselin, I, Chamberlain, J, and Tremblay, J P (1998). Transplantation of myoblasts from a transgenic mouse overexpressing dystrophin prduced only a relatively small increase of dystrophin-positive membrane. Muscle Nerve 21: 91-103.
55. Pavlath, G K, Rich, K, Webster, S G, and Blau, H M (1989). Localization of muscle gene products in nuclear domains. Nature 337: 570-573.
56. Nicolas, A, Raguenes-Nicol, C, Ben Yaou, R, Ameziane-Le Hir, S, Cheron, A, Vie, V, et al. (2015). Becker muscular dystrophy severity is linked to the structure of dystrophin. Hum Mol Genet 24: 1267-1279.
57. Kaspar, R W, Allen, H D, Ray, W C, Alvarez, C E, Kissel, J T, Pestronk, A, et al. (2009). Analysis of dystrophin deletion mutations predicts age of cardiomyopathy onset in becker muscular dystrophy. Circ Cardiovasc Genet 2: 544-551.
58. Ran, F A, Hsu, P D, Wright, J, Agarwala, V, Scott, D A, and Zhang, F (2013). Genome engineering using the CRISPR-Cas9 system. Nat Protoc 8: 2281-2308.
59. t Hoen, P A, de Meijer, E J, Boer, J M, Vossen, R H, Turk, R, Maatman, R G, et al. (2008). Generation and characterization of transgenic mice with the full-length human DMD gene. J Biol Chem 283: 5899-5907.
60. Mohanraju, P. et al. (2016). Diverse evolutionary roots and mechanistic variations of the CRISPR-Cas systems. Science 353(6299:aad5147.
61. Shmakov, S et al. (2015). Discovery and Functional Characterization of Diverse Class 2 CRISPR-Cas Systems. Mol Cell 60(3):385-97.
62. Zetsche, B. et al. (2015). Cpf1 is a single RNA-guided endonuclease of a class 2 CRISPR-Cas system. Cell 163(3):759-71.
63. Kleinstiver, B P. et al. (2015). Broadening the targeting range of Staphylococcus aureus CRISPR-Cas9 by modifying PAM recognition. Nat Biotechnol 33(12):1293-1298.
64. Tsai S Q. et al. (2014). Dimeric CRISPR RNA-guided FokI nucleases for highly specific genome editing. Nature Biotechnology, 32, 569-576.
65. Koo T. et al. (2015). Measuring and Reducing Off-Target Activities of Programmable Nucleases Including CRISPR-Cas9. Mol Cells 38(6):475-481.

1-68. (canceled)

69. A method of modifying a dystrophin gene and restoring the correct reading frame for dystrophin expression within a cell having an endogenous frameshift or nonsense mutation within the dystrophin (DYS) gene, the method comprising:
a) introducing a first cut within an exon or an intron of the DYS gene creating a first exon end or intron end, wherein said first cut is located upstream of the endogenous frameshift or nonsense mutation;
b) introducing a second cut within an exon or an intron of the DYS gene creating a second exon end or second intron end, wherein said second cut is located downstream of the frameshift or nonsense mutation;
wherein upon ligation of said first and second exon ends or said first and second intron ends, a modified dystrophin gene comprising a hybrid exon or intron is created and dystrophin expression is restored.

70. The method of claim 69, wherein said first and second cuts are introduced by providing a cell with i) a CRISPR nuclease; and ii) a pair of gRNAs consisting of a) a first gRNA which binds to an exon or intron sequence of the DYS gene located upstream of the endogenous frameshift or nonsense mutation for introducing the first cut; b) a second gRNA which binds to an exon or intron sequence of the DYS gene located downstream of the endogenous frameshift or nonsense mutation for introducing the second cut.

71. The method of claim 69, wherein the endogenous frameshift or nonsense mutation is located within a region of the dystrophin gene encompassing exons 45-58 of the dystrophin gene.

72. The method of claim 69, wherein the first cut is within exon 46, 47, 48, 49 or 50 and the second cut is within exon 51, 52, 53, 54, 55, 56, 57 or 58, of the dystrophin gene.

73. The method of claim 69, wherein said modified dystrophin gene encodes a modified dystrophin protein comprising a hybrid spectrin-like repeat (SLR) comprising a portion of a first SLR and a portion of second SLR, thereby comprising a hybrid SLR junction, and wherein the wild-type configuration of said hybrid SLR is maintained where hydrophobic amino acids are localized in position “a” and “d” of the heptad motif.

74. A gRNA pair for restoring dystrophin expression in a cell comprising an endogenous frameshift or nonsense mutation within the dystrophin (DYS) gene, wherein said pair consists of a first gRNA and a second gRNA, wherein said first gRNA comprises a first target sequence upstream of the endogenous frameshift or nonsense mutation and can direct a nuclease-mediated first cut in an exon or intron sequence of the DYS gene located upstream of the endogenous frameshift or nonsense mutation and wherein said second gRNA comprises a second target sequence downstream of the endogenous frameshift or nonsense mutation and can direct a nuclease-mediated second cut in an exon or intron sequence of the DYS gene located downstream of the endogenous frameshift or nonsense mutation.

75. The gRNA pair of claim 74, wherein the endogenous frameshift or nonsense mutation is located within a region of the dystrophin gene encompassing exons 45-58 of the dystrophin gene.

76. The gRNA pair of claim 74, wherein the first cut is within exon 46, 47, 48, 49 or 50 and the second cut is within exon 51, 52, 53, 54, 55, 56, 57 or 58, of the dystrophin gene.

77. The gRNA pair of claim 74, wherein said gRNA pair allows to generate a modified dystrophin gene encoding a modified dystrophin protein comprising a hybrid spectrin-like repeat (SLR) comprising a portion of a first SLR and a portion of second SLR and a hybrid SLR junction and wherein the hybrid SLR has a wild-type configuration where hydrophobic amino acids are localized in position “a” and “d” of the heptad motif.

78. A nucleic acid comprising one or more sequences encoding one or both members of the gRNA pair of claim 74.

79. A nucleic acid comprising a modified dystrophin gene comprising ligated first and second exon ends or first and second intron ends as defined in claim 69.

80. A modified dystrophin polypeptide encoded by the nucleic acid of claim 79.

81. A vector comprising the nucleic acid of claim 78.

82. A cell comprising one or both members of the gRNA pair of claim 74.

83. A composition comprising one or both members of the gRNA pair of claim 74.

84. A composition comprising the vector of claim 81.

85. A kit comprising one or both members of the gRNA pair claim 74.

86. A method for treating muscular dystrophy in a subject, comprising modifying a dystrophin gene and restoring the correct reading frame for dystrophin expression within a cell of said subject according to the method of claim 69.

87. A method for treating muscular dystrophy in a subject, comprising contacting a cell of the subject with (i)(a) the gRNA pair of claim 74 or one or more nucleic acids encoding said gRNA pair and (b) a CRISPR nuclease polypeptide or a nucleic acid encoding a CRISPR nuclease polypeptide.

88. A reaction mixture comprising (a) the gRNA pair of claim 74 or one or more nucleic acids encoding said gRNA pair and (b) a CRISPR nuclease polypeptide or a nucleic acid encoding a CRISPR nuclease polypeptide.
